SEQUENCE LISTING (13 sequences)

SEQ ID NO: 1 - DNA, 948 bp, Mus musculus
attaatatcctaacactcctcgtccccattctaatcgccatagccttcctaacattagtagaacgcaaaatcttagggtacatacaactacgaaaaggccctaacattgttggtccatacggcattttacaaccatttgcagacgccataaaattatttataaaagaaccaatacgccctttaacaacctctatatccttatttattattgcacctaccctatcactcacactagcattaagtctatgagttcccctaccaataccacacccattaattaatttaaacctagggattttatttattttagcaacatctagcctatcagtttactccattctatgatcaggatgagcctcaaactccaaatactcactattcggagctttacgagccgtagcccaaacaatttcatatgaagtaaccatagctattatccttttatcagttctattaataaatggatcctactctctacaaacacttattacaacccaagaacacatatgattacttctgccagcctgacccatagccataatatgatttatctcaaccctagcagaaacaaaccgggcccccttcgacctgacagaaggagaatcagaattagtatcagggtttaacgtagaatacgcagccggcccattcgcgttattctttatagcagagtacactaacattattctaataaacgccctaacaactattatcttcctaggacccctatactatatcaatttaccagaactctactcaactaacttcataatagaagctctactactatcatcaacattcctatggatccgagcatcttatccacgcttccgttacgatcaacttatacatcttctatgaaaaaactttctacccctaacactagcattatgtatgtgacatatttctttaccaatttttacagcgggagtaccaccatacatatag

SEQ ID NO: 2 - DNA, 1038 bp, Mus musculus
ataaatcctatcacccttgccatcatctacttcacaatcttcttaggtcctgtaatcacaatatccagcaccaacctaatactaatatgagtaggcctagaattcagcctactagcaattatccccatactaatcaacaaaaaaaacccacgatcaactgaagcagcaacaaaatacttcgtcacacaagcaacagcctcaataattatcctcctggccatcgtactcaactataaacaactaggaacatgaatatttcaacaacaaacaaacggtcttatccttaacataacattaatagccctatccataaaactaggcctcgccccattccacttctgattaccagaagtaactcaagggatcccactgcacataggacttattcttcttacatgacaaaaaattgctcccctatcaattttaattcaaatttacccgctactcaactctactatcattttaatactagcaattacttctattttcataggggcatgaggaggacttaaccaaacacaaatacgaaaaattatagcctattcatcaattgcccacataggatgaatattagcaattcttccttacaacccatccctcactctactcaacctcataatctatattattcttacagcccctatattcatagcacttatactaaataactctataaccatcaactcaatctcacttctatgaaataaaactccagcaatactaactataatctcactgatattactatccctaggaggccttccaccactaacaggattcttaccaaaatgaattatcatcacagaacttataaaaaacaactgtctaattatagcaacactcatagcaataatagctctactaaacctattcttttatattcgcctaatttattccacttcactaacaatatttccaaccaacaataactcaaaaataataactcaccaaacaaaaactaaacccaacctaatattttccaccctagctatcataagcacaataaccctacccctagccccccaactaattacctag

SEQ ID NO: 3 - DNA, 1545 bp, Mus musculus
atgttcattaatcgttgattattctcaaccaatcacaaagatatcggaaccctctatctactattcggagcctgagcgggaatagtgggtactgcactaagtattttaattcgagcagaattaggtcaaccaggtgcacttttaggagatgaccaaatttacaatgttatcgtaactgcccatgcttttgttataattttcttcatagtaataccaataataattggaggctttggaaactgacttgtcccactaataatcggagccccagatatagcattcccacgaataaataatataagtttttgactcctaccaccatcatttctccttctcctagcatcatcaatagtagaagcaggagcaggaacaggatgaacagtctacccacctctagccggaaatccagtccatgcaggagcatcagtagacctaacaattttctcccttcatttagctggagtgtcatctattttaggtgcaattaattttattaccactattatcaacatgaaacccccagccataacacagtatcaaactccactatttgtctgatccgtacttattacagccgtactgctcctattatcactaccagtgctagccgcaggcattactatactactaacagaccgcaacctaaacacaactttctttgatcccgctggaggaggggacccaattctctaccagcatctgttctgattctttgggcacccagaagtttatattcttatcctcccaggatttggaattatttcacatgtagttacttactactccggaaaaaaagaacctttcggctatataggaatagtatgagcaataatgtctattggctttctaggctttattgtatgagcccaccacatattcacagtaggattagatgtagacacacgagcttgctttacatcagccactataattatcgcaattcctaccggtgtcaaagtatttagctgacttgcaaccctacacggaggtaatattaaatgatctccagctatactatgagccttaggctttattttcttatttacagttggtggtctaaccggaattgttttatccaactcatcccttgacatcgtgcttcacgatacatactatgtagtagcccatttccactatgttctatcaatgggagcagtgtttgctatcatagcaggatttgttcactgattcccattattttcaggcttcaccctagatgacacatgagcaaaagcccacttcgccatcatattcgtaggagtaaacataacattcttccctcaacatttcctgggcctttcaggaataccacgacgctactcagactacccagatgcttacaccacatgaaacactgtctcttctataggatcatttatttcactaacagctgttctcatcatgatctttataatttgagaggcctttgcttcaaaacgagaagtaatatcagtatcgtatgcttcaacaaatttagaatgacttcatggctgccctccaccatatcacacattcgaggaaccaacctatgtaaaagtaaaataa

SEQ ID NO: 4 - DNA, 684 bp, Mus musculus
atggcctacccattccaacttggtctacaagacgccacatcccctattatagaagagctaataaatttccatgatcacacactaataattgttttcctaattagctccttagtcctctatatcatctcgctaatattaacaacaaaactaacacatacaagcacaatagatgcacaagaagttgaaaccatttgaactattctaccagctgtaatccttatcataattgctctcccctctctacgcattctatatataatagacgaaatcaacaaccccgtattaaccgttaaaaccatagggcaccaatgatactgaagctacgaatatactgactatgaagacctatgctttgattcatatataatcccaacaaacgacctaaaacctggtgaactacgactgctagaagttgataaccgagtcgttctgccaatagaacttccaatccgtatattaatttcatctgaagacgtcctccactcatgagcagtcccctccctaggacttaaaactgatgccatcccaggccgactaaatcaagcaacagtaacatcaaaccgaccagggttattctatggccaatgctctgaaatttgtggatctaaccatagctttatgcccattgtcctagaaatggttccactaaaatatttcgaaaactgatctgcttcaataatttaa

SEQ ID NO: 5 - DNA, 204 bp, Mus musculus
atgccacaactagatacatcaacatgatttatcacaattatctcatcaataattaccctatttatcttatttcaactaaaagtctcatcacaaacattcccactggcaccttcaccaaaatcactaacaaccataaaagtaaaaaccccttgagaattaaaatgaacgaaaatctatttgcctcattcattaccccaacaataa

SEQ ID NO: 6 - DNA, 681 bp, Mus musculus
atgaacgaaaatctatttgcctcattcattaccccaacaataataggattcccaatcgttgtagccatcattatatttccttcaatcctattcccatcctcaaaacgcctaatcaacaaccgtctccattctttccaacactgactagttaaacttattatcaaacaaataatgctaatccacacaccaaaaggacgaacatgaaccctaataattgtttccctaatcatatttattggatcaacaaatctcctaggccttttaccacatacatttacacctactacccaactatccataaatctaagtatagccattccactatgagctggagccgtaattacaggcttccgacacaaactaaaaagctcacttgcccacttccttccacaaggaactccaatttcactaattccaatacttattattattgaaacaattagcctatttattcaaccaatggcattagcagtccggcttacagctaacattactgcaggacacttattaatacacctaatcggaggagctactctagtattaataaatattagcccaccaacagctaccattacatttattattttacttctactcacaattctagaatttgcagtagcattaattcaagcctacgtattcaccctcctagtaagcctatatctacatgataatacataa

SEQ ID NO: 7 - DNA, 784 bp, Mus musculus
atgacccaccaaactcatgcatatcacatagttaatccaagtccatgaccattaactggagccttttcagccctccttctaacatcaggtctagtaatatgatttcactataattcaattacactattaacccttggcctactcaccaatatcctcacaatatatcaatgatgacgagacgtaattcgtgaaggaacctaccaaggccaccacactcctattgtacaaaaaggactacgatatggtataattctattcatcgtctcggaagtatttttctttgcaggattcttctgagcgttctatcattctagcctcgtaccaacacatgatctaggaggctgctgacctccaacaggaatttcaccacttaaccctctagaagtcccactacttaatacttcagtacttctagcatcaggtgtttcaattacatgagctcatcatagccttatagaaggtaaacgaaaccacataaatcaagccctactaattaccattatactaggactttacttcaccatcctccaagcttcagaatactttgaaacatcattctccatttcagatggtatctatggttctacattcttcatggctactggattccatggactccatgtaattattggatcaacattccttattgtttgcctactacgacaactaaaatttcacttcacatcaaaacatcacttcggatttgaagccgcagcatgatactgacattttgtagacgtaatctgacttttcctatacgtctccatttattgatgaggatctt

SEQ ID NO: 8 - DNA, 345 bp, Mus musculus
atcaacctgtacactgttatcttcattaatattttattatccctaacgctaattctagttgcattctgactcccccaaataaatctgtactcagaagcaaatccatatgaatgcggattcgaccctacaagctctgcacgtctaccattctcaataaaatttttcttggtagcaattacatttctattatttgacctagaaattgctcttctacttccactaccatgagcaattcaaacaattaaaacctctactataataattatagcctttattctagtcacaattctatctctaggcctagcatatgaatgaacacaaaaaggattagaatgaacagagtaa

SEQ ID NO: 9 - DNA, 294 bp, Mus musculus
atgccatctaccttcttcaacctcaccatagccttctcactatcacttctagggacacttatatttcgctctcacctaatatccacattactatgcctggaaggcatagtattatccttatttattataacttcagtaacttccctaaactccaactccataagctccataccaatccccatcaccttagttttcgcagcctgcgaagcagctgtaggactagccctactagtaaaagtttcaaacacgtacggaacagattacgtccaaaatctcaacctactacaatgctaa

SEQ ID NO: 10 - DNA, 1378 bp, Mus musculus
atgctaaaaattattcttccctcactaatgctactaccactaacctgactatcaagccctaaaaaaacctgaacaaacgtaacctcatatagttttctaattagtttaaccagcctaacacttctatgacaaaccgacgaaaattataaaaacttttcaaatatattctcctcagaccccctatccacaccattaattattttaacagcctgattactgccactaatattaatagctagccaaaaccacctaaaaaaagataataacgtactacaaaaactctacatctcaatactaatcagcttacaaattctcctaatcataaccttttcagcaactgaactaattatattttatattttatttgaagcaaccttaatcccaacacttattattattacccgatgagggaaccaaactgaacgcctaaacgcagggatttatttcctattttataccctaatcggttctattccactgctaattgccctcatcttaatccaaaaccatgtaggaaccctaaacctcataattttatcattcacaacacacaccttagacgcttcatgatctaacaacttactatggttggcatgcataatagcatttcttattaaaataccattatatggagttcacctatgactaccaaaagcccatgttgaagctccaattgctgggtcaataattctagcagctattcttctaaaattaggtagttacggaataattcgcatctccattattctagacccactaacaaaatatatagcataccccttcatccttctctccctatgaggaataattataactagctcaatctgcttacgccaaacagatttaaaatcactaatcgcctactcctcagttagccacatagcacttgttattgcatcaatcataatccaaactccatgaagcttcataggagcaacaatactaataatcgcacatggcctcacatcatcactcctattctgcctagcaaactccaactacgaacggatccacagccgtactataatcatggcccgaggacttcaaatggtcttcccacttatagccacatgatgactgatagcaagtctagctaatctagctctacccccttcaatcaatctaataggagaattattcattaccatatcattattttcttgatcaaactttaccattattcttataggaattaacattattattacaggtatatactcaatatacataattattaccacccaacgcggcaaactaaccaaccatataattaacctccaaccctcacacacacgagaactaacactaatagcccttcacataattccacttattcttctaactaccagtccaaaactaattacaggcctgacaatat

SEQ ID NO: 11 - DNA, 1824 bp, Mus musculus
atcaatattttcacaacctcaatcttattaatcttcattcttctactatccccaatcctaatttcaatatcaaacctaattaaacacatcaacttcccactgtacaccaccacatcaatcaaattctccttcattattagcctcttacccctattaatatttttccacaataatatagaatatataattacaacctggcactgagtcaccataaattcaatagaacttaaaataagcttcaaaactgactttttctctatcctgtttacatctgtagccctttttgtcacatgatcaattatacaactctcttcatgatatatacactcagacccaaacatcaatcgattcattaaatatcttacactattcctgattaccatgcttatcctcacctcagccaacaacatatttcaacttttcattggctgagaaggggtgggaattatatctttcctactaattggatgatggtacggacgaacagacgcaaatactgcagccctacaagcaatcctctataaccgcatcggagacatcggattcattttagctatagtttgattttccctaaacataaactcatgagaacttcaacagattatattctccaacaacaacgacaatctaattccacttataggcctattaatcgcagctacaggaaaatcagcacaatttggcctccacccatgactaccatcagcaatagaaggccctacaccagtttcagcactactacactcaagtacaatagtagttgcaggaattttcctactggtccgattccaccccctcacgactaataataactttattttaacaactatactttgcctcggagccctaaccacattatttacagctatttgtgctctcacccaaaacgacatcaaaaaaatcattgccttctctacatcaagccaactaggcctgataatagtgacgctaggaataaaccaaccacacctagcattcctacacatctgtacccacgcattcttcaaagctatactctttatatgctctggctcaatcattcatagcctggcagacgaacaagacatccgaaaaataggaaacatcacaaaaatcataccattcacatcatcatgcctagtaatcggaagcctcgccctcacaggaataccattcctaacagggttctactcaaaagacctaattattgaagcaattaatacctgcaacaccaacgcctgagccctactaattacactaatcgccacttctataacagctatgtacagcatacgaatcatttacttcgtaacaataacaaaaccgcgttttccccccctaatctccattaacgaaaatgacccagacctcataaacccaatcaaacgcctagcattcggaagcatctttgcaggatttgtcatctcatataatattccaccaaccagcattccagtcctcacaataccatgatttttaaaaaccacagccctaattatttcagtattaggattcctaatcgcactagaactaaacaacctaaccataaaactatcaataaataaagcaaatccatattcatccttctcaactttactggggtttttcccatctattattcaccgcattacacccataaaatctctcaacctaagcctaaaaacatccctaactctcctagacttgatctggttagaaaaaaccatcccaaaatccacctcaactcttcacacaaacataaccactttaacaaccaaccaaaaaggcttaattaaattgtactttatatcattcctaattaacatcatcttaattattatcttatactcaattaatctcgagtaa

SEQ ID NO: 12 - DNA, 519 bp, Mus musculus
atgaataattatatttttgttttaagttcattatttttggttggttgtcttgggttagcattaaagccttcacctatttatggaggtttaggtttaattgttagtgggtttgttggttgtttaatggttttagggtttggtggatcgtttttaggtttaatagtttttttaatttatttaggggggatgttggttgtgtttggatatacgactgctatagctactgaggaatatccagagacttggggatctaactgattaattttgggttttttagtattgggggtgattatagaggtttttttaatttgtgtgcttaattattatgatgaagttggagtaattaatcttgatggtttgggagattggttgatgtatgaggttgatgatgttggagttatgttggaaggagggattggggtagcggcaatatatagttgtgctacttgaatgatggtagtagctgggtgatctttgtttgcgggtatttttattattatcgagattactcgagattaa

SEQ ID NO: 13 - DNA, 1144 bp, Mus musculus
atgacaaacatacgaaaaacacacccattatttaaaattattaaccactcattcattgacctacctgccccatccaacatttcatcatgatgaaactttgggtcccttctaggagtctgcctaatagtccaaatcattacaggtcttttcttagccatacactacacatcagatacaataacagccttttcatcagtaacacacatttgtcgagacgtaaattacgggtgactaatccgatatatacacgcaaacggagcctcaatattttttatttgcttattccttcatgtcggacgaggcttatattatggatcatatacatttatagaaacctgaaacattggagtacttctactgttcgcagtcatagccacagcatttataggctacgtccttccatgaggacaaatatcattctgaggtgccacagttattacaaacctcctatcagccatcccatatattggaacaaccctagtcgaatgaatttgagggggcttctcagtagacaaagccaccttgacccgattcttcgctttccacttcatcttaccatttattatcgcggccctagcaatcgttcacctcctcttcctccacgaaacaggatcaaacaacccaacaggattaaactcagatgcagataaaattccatttcacccctactatacaatcaaagatatcctaggtatcctaatcatattcttaattctcataaccctagtattatttttcccagacatactaggagacccagacaactacataccagctaatccactaaacaccccaccccatattaaacccgaatgatatttcctatttgcatacgccattctacgctcaatccccaataaactaggaggtgtcctagccttaatcttatctatcctaattttagccctaatacctttccttcatacctcaaagcaacgaagcctaatattccgcccaatcacacaaattttgtactgaatcctagtagccaacctacttatcttaacctgaattgggggccaaccagtagaacacccatttattatcattggccaactagcctccatctcatacttctcaatcatcttaattcttataccaatctcaggaattatcgaagacaaaatactaaaattatatccat

(19) United States
(12) Patent Application Publication
(10) Pub. No.: US 2008/0187911 A1
(43) Pub. Date: Aug. 7, 2008

(54) Methods and compositions for analysis of mitochondrial-related gene expression

(75) Inventors: John Papaconstantinou, Galveston, TX (US); James Deford, Galveston, TX (US); Arpad Gerstner, Galveston, TX (US)

Correspondence Address: FULBRIGHT & JAWORSKI L.L.P., 600 CONGRESS AVE., SUITE 2400, AUSTIN, TX, 78701, US

(73) Assignee: Research Development Foundation

(21) Appl. No.: 10/767,217

(22) Filed: Jan. 29, 2004

Related U.S. Application Data
(60) Provisional application No. 60/443,681, filed on Jan. 30, 2003.

(51) Int. Cl. C12Q 1/68; C40B 40/06; C40B 30/00

(52) U.S. Cl. 435/6; 506/16; 506/7

(57) ABSTRACT

The invention provides arrays for analyzing the expression of mitochondrial-related coding sequences. The invention allows the efficient analysis of expression levels across each of these coding sequences. The invention has important applications in the field of medicine for the screening and diagnosis of patients with ailments associated with aberrant mitochondrial function, as well as in the development of treatments therefore.

The present application claims priority to co-pending U.S. Provisional Patent Application Ser. No. 60/443,681 filed Jan. 30, 2003. The entire text of the above-referenced disclosure is specifically incorporated herein by reference without disclaimer.

The government may own rights in the present invention pursuant to grant number Grant No. P60AG17231 from the National Institutes of Health, National Institute on Aging.

### BACKGROUND OF THE INVENTION

1. Field of the Invention

The present invention relates generally to the fields of molecular biology and medicine. More particularly, the invention relates to arrays of nucleic acids immobilized on a solid support for selectively monitoring expression of mitochondrial-related genes from the nuclear and mitochondrial genomes and methods for the use thereof.

2. Description of Related Art

Global populations of individuals over the age of 65 have increased, with most destined to live into their 80s. Given the average survival age of the elderly, improvements in the health of the elderly are needed or the economy will be faced with a tremendous burden. The economy will be burdened with special needs for nursing care, transportation, housing, and medical arrangements. This burden can be reduced by improving overall health care. Substantial increases in research on diseases of aging are thus needed. Currently, less than one percent of the 1.14 trillion dollars the U.S. spends each year on health care goes for research on Alzheimer's, arthritis, Parkinson's, prostate cancer and other age-related diseases. Unless more diseases of aging are delayed or conquered, mounting bills for illness will swamp even the most robust Medicare program.

Finding cures and alleviating symptoms of diseases would have a major positive effect on the economy. According to studies by the Milken Institute, an investment of 175 million dollars in diabetes research now saves 7 billion dollars in medical costs. Work done by the University of Chicago supports this thinking, with studies reporting that the economic value of reductions in heart disease in people aged 70 to 80 could amount to 15 trillion dollars. Also, as exemplified by the work of others, diseases such as polio, Alzheimer's and many other aging and age-related diseases can be conquered. Thus, research can do much to improve the quality of life for the elderly.

A major key to understanding, alleviating, or ameliorating diseases of the aging population lies in the genetic basis of aging. The sequence of the entire human genome Anderson et al., 1981) has been completed and will greatly advance the development of technologies beneficial in understanding the genetic basis of aging. The sequence of the entire mouse genome has recently been reported and will advance biomedical research on animal models representative of human diseases (Waterston, et al., 2002). Studies at UTMB Galveston have recently shown that mitochondrial (mtDNA) is damaged three to four times more frequently than nuclear DNA by a wide variety of agents, which induce reactive oxygen species (Mandavilli et al. 2002; Santos et al., 2002; Ballinger et al., 2000). Thus, mitochondrial DNA and its ability to transcribe mitochondrial specific genes represent a critical cellular target for reactive oxygen species-induced cell death.

There are two major hypotheses that deal with the role of mitochondrial integrity and function in aging: firstly, the catastrophic demise of mitochondrial function is a primary mechanism in aging; and secondly, ROS generated in the mitochondria causes mitochondrial DNA damage, which in turn causes the release of more ROS, leading to further mitochondrial decline and age-associated pathologies (Harmon, 1972; Golden and Melov, 2001; Ames et al., 1993; Finkel and Holbrook, 2000; Beckman and Ames, 1998; Beckman and Ames, 1999; Zhang et al., 1992).

Therefore, the integrity of the mitochondria is a major factor in the function of aged tissues, mitochondria-associated diseases, and responses of the mitochondria to oxidative stress or inflammatory agents—both environmental and internal. The mitochondrion provides the energy needed to carry out critical biological functions. Any factor(s) that disrupt or compromise mitochondrial functions are of importance, because they relate to diseases including genetic diseases, environmental toxins, and responses to hormones and growth factors (Mitochondria and Free radicals in Neurodegenerative Diseases, 1997).

Most human genes are encoded by the nuclear DNA of the cell, but some are also found in the mitochondrial DNA. Mitochondria are the “power plants” within each cell and provide about 90 percent of the energy necessary for cells—and thus provide tissues, organs and the body as a whole with energy. Mutations of the mtDNA can cause a wide range of disorders—from neurodegenerative diseases to diabetes and heart failure. Scientists also suspect that injury to the genes within the mitochondria may play an important role in the aging process as well as in chronic degenerative illnesses, such as Alzheimer's Parkinson's and Lou Gehric's disease (Golden and Melov, 2001; Ames et al., 1993).

In the course of investigating mtDNA deletions in disease it became apparent that normal individuals can also be heteroplasmic for deleted mtDNA and that the fraction of deleted DNA increases exponentially with age. These observations raised interest in the role played by mtDNA mutations in aging. One hypothesis is that continuous oxidative damage to mtDNA is responsible for an age-related decline in oxidative phosphorylation capacity (Golden and Melov, 2001; Finkel and Holbrook, 2001; Ventura et al. 2002). Whether a causal relationship exists between mtDNA mutations and aging, however, remains to be established.

What has been lacking in the art is a procedure allowing simultaneous and parallel determination of the activity of mitochondrial and nuclear genes that make the enzymes and structural protein of the mitochondrion. Analysis of the mRNA levels of each of these genes would provide insight as to the overall biochemical phenotype (picture) of mitochondrial organellogenesis. Procedures have been available to determine the activity of a limited numbers of genes in one experiment. There are, however, several hundred mitochondrial-related genes. What is needed, therefore, is a method of analyzing the expression of these genes, thereby providing insight as to the roles mitochondrial proteins play in different disease states.

### SUMMARY OF THE INVENTION

The invention overcomes the deficiencies in the art by providing methods and compositions for assessing the integrity and function of the mitochondria. Thus, the invention provides arrays comprising nucleic acid molecules comprising a plurality of sequences, wherein the molecules are immobilized on a solid support and wherein at least 5% of the immobilized molecules are capable of hybridizing to mitochondrial-related acid sequences or complements thereof.

In some aspects of the invention, the array may further be defined as comprising at least 20, at least 40, at least 100, at least 200, or at least 400 nucleic acid molecules. In other aspects the array of the invention comprises nucleic acid molecules comprising cDNA sequences. In further aspects of the invention, the nucleic acid molecules may comprise at least 17 nucleotides. These mitochondrial-related nucleic acid sequences may, for example, be from a mammal, a primate, a human, a mouse, a yeast, an arthropod such as a Drosophila, or a nematode such as C. elegans. In certain embodiments of the invention, at least 25%, at least 35%, at least 50%, at least 75%, at least 85%, at least 95%, or at least 100% of the immobilized molecules are capable of hybridizing to mitochondrial-related nucleic acid sequences or complements thereof. In still a further aspect of the invention, at least one of the mitochondrial-related nucleic acid sequences is encoded by a mitochondrial genome.

In particular aspects of the invention, the immobilized molecules are capable of hybridizing to at least 5, at least 10, at least 15, at least 30, at least 60, at least 100, or at least 200 mitochondrial-related nucleic acid sequences or complements thereof. In further aspects of the invention, the immobilized molecules are capable of hybridizing to at least 300, at least 500, or at least 1000 mitochondrial-related nucleic acid sequences or complements thereof. In further aspects of the invention, at least one of the mitochondrial-related nucleic acid sequences is encoded by a nuclear or mitochondrial genome.

In a further aspect, the invention provides a method for measuring the expression of one or more mitochondrial-related coding sequence in a cell or tissue, the method comprising: a) contacting an array as described above with a sample of nucleic acids from the cell or tissue under conditions effective for mRNA or complements thereof from the cell or tissue to hybridize with the nucleic acid molecules immobilized on the solid support; and b) detecting the amount of mRNA or complements thereof hybridizing to mitochondrial-related nucleic acid sequences or complements thereof. In one embodiment of the invention, the detecting in step (b) may be carried out calorimetrically, fluorometrically, or radiometrically. In certain embodiments, the cell may be a mammal cell, a primate cell, a human cell, a mouse cell, or an yeast cell.

In yet another aspect, the invention provides a method of screening an individual for a disease state associated with altered expression of one or more mitochondrial-related nucleic acid sequences comprising: a) contacting an array, according to that described above, with a sample of nucleic acids from the individual under conditions effective for the mRNA or complements thereof from the individual to hybridize with the nucleic acid molecules immobilized on the solid support; b) detecting the amount of mRNA or complements thereof hybridizing to mitochondrial-related nucleic acid sequences; and c) screening the individual for a disease state by comparing the expression of the mitochondrial-related nucleic acid sequences detected with a pattern of expression of the mitochondrial-related nucleic acid sequences associated with the disease state. In one embodiment of the invention, the disease state may be selected from that provided in Table 1. In particular aspects, the disease state is cystic fibrosis, Alzheimer's disease, Parkinson's disease, ataxia, Wilson disease, Maple syrup urine disease, Barth syndrome, Leber's hereditary optic neuropathy, congenital adrenal hyperplasia diabetes mellitus, multiple sclerosis, or cancer, but is not limited to such.

In one embodiment of the invention, detecting the amount of mRNA or complements thereof hybridizing to mitochondrial-related nucleic acid sequences may be carried out calorimetrically, fluorometrically, or radiometrically. In further aspects of the invention, the individual may be a mammal, a primate, a human, a mouse, an arthropod, or an nematode but is not limited to such.

In still yet another aspect, the invention provides a method of screening a compound for its affect on mitochondrial structure and/or function comprising: a) contacting an array according to that described above, with a sample of nucleic acids from a cell under conditions effective for the mRNA or complements thereof from the cell to hybridize with the nucleic acid molecules immobilized on the solid support, wherein the cell has previously been contacted with the compound under conditions effective to permit the compound to have an affect on mitochondrial structure and/or function; b) detecting the amount of mRNA encoded by mitochondrial-related nucleic acid sequences or complements thereof that hybridizes with the nucleic acid molecules immobilized on the solid support; and c) correlating the detected amount of mRNA encoded by mitochondrial-related nucleic acid molecules or complements thereof with the affect of the compound mitochondrial structure and/or function.

In one embodiment of the invention, the compound is a small molecule. In another embodiment of the invention, the compound is formulated in a pharmaceutically acceptable carrier or diluent. In still another embodiment of the invention, the compound may be an oxidative stressing agent, an inflammatory agent, or a chemotherapeutic agent.

In still yet another aspect, the present invention provides a method for screening an individual for reduced mitochondrial function comprising: a) contacting an array according to that described above, with a sample of nucleic acids from a cell under conditions effective for the mRNA or complements thereof from the cell to hybridize with the nucleic acid molecules immobilized on the solid support; b) detecting the amount of mRNA encoded by mitochondrial-related nucleic acid sequences or complements thereof that hybridizes with the nucleic acid molecules immobilized on the solid support; and c) correlating the detected amount of mRNA or complements thereof with reduced mitochondrial function.

In certain embodiments of the invention, the detecting step as described above may be carried out calorimetrically, fluorometrically, or radiometrically. In still another embodiment, the individual is a mammal, a primate, a human, a mouse, an arthropod, or a nematode.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. DNA microarray generated from PCR™ products using thirteen genes that code for mitochondrial proteins.

FIG. 2. Map of the Mus musculus mitochondrial DNA showing the location of the 13 peptides of the OXPHOS complexes.

FIG. 3. Map of the Homo sapien mitochondrial DNA showing the location of the 13 peptides of the OXPHOS complexes.

FIG. 4. The effects of rotenone, an inhibitor of mitochondrial Complex I, on the expression of mouse mitochondrial genes in AML-12 mouse liver cells in culture.

FIGS. 5A-5B. Analysis of mitochondrial DNA encoded gene expression. FIG. 5A—response to 3-nitropropionic acid, an inhibitor of Complex II—succinic dehydrogenase. The data show that inhibition of Complex II stimulates the synthesis of mitochondrial encoded mRNAs and the 23S and 16S ribosomal RNAs. FIG. 5B-analysis of mitochondrial DNA encoded gene expression in trypanosome infected heart tissue. The data show a decline in mRNA and ribosomal RNA levels at 37 days post infection.

FIGS. 6A-6C. Analysis of mitochondrial gene expression in mouse mutants. FIG. 6A—mitochondrial gene expression in livers of young Snell dwarf mouse mutants. FIG. 6B—analysis of mitochondrial gene expression in livers of aged Snell dwarf mouse mutants. FIG. 6C—RT-PCR analysis of Hsd3b5 expression levels in control versus dwarf Snell mice.

FIGS. 7A-7D. Analysis of mitochondrial gene expression in heart muscle of trypanosome infected mice. FIG. 7A—control; FIGS. 7B-7D—three heart muscles from trypanosome infected mice.

FIGS. 8A-8D. The effects of 40% TBS thermal injury on mouse liver mitochondrial function in control (FIG. 8A) and three livers from thermally injured mice 24 hours after burn (FIGS. 8B-8D).

FIG. 9. Array analysis of the expression of the 13 mitochondrial DNA encoded genes in livers of thermally injured mice.

### DESCRIPTION OF ILLUSTRATIVE EMBODIMENTS

The present invention overcomes limitations in the art by providing methods and compositions for determining the integrity and function of the mitochondria. Arrays are provided that allow simultaneous screening of the expression of mitochondrial-related coding sequences. The invention thus allows determination of the role of mitochondrial genes in various disease states. The ability to accumulate gene expression data for the mitochondria provides a powerful opportunity to assign functional information to genes of otherwise unknown function. The conceptual basis of the approach is that genes that contribute to the same biological process will exhibit similar patterns of expression. This mitochondrial gene array thus provides insight into the development and treatment of disease states associated with effects on mitochondrial structure and/or function.

### A. The Present Invention

Use of arrays, including microarrays and gene chips, provides a promising approach for uncovering mitochondrial gene function. A major factor in the age-associated gradual decline of tissue function has been attributed to the reduction or loss of mitochondrial integrity and function. Furthermore, this has been attributed to the age-associated increase in oxidative stress that targets mitochondrial DNA and proteins. One aspect of the present invention is thus to determine the integrity of the mitochondria, both structure and function, as is indicated by the activity of the genes that code for mitochondrial enzymes and structural proteins.

Another aspect of the present invention is to identify the genetic expression patterns that govern aging. The mtDNA array can be used to determine specific patterns of altered gene expression for mtDNA as well as the nuclear DNA that encodes the mitochondrial proteins. In order to achieve this goal, mitochondrial and related nuclear genes can be used to generate an array of nucleic acids by immobilizing them on a solid support, including, but not limited to, a microscopic slide or hybridization filter. By screening a plurality of mitochondrial-related coding sequences (genes) in this manner, associations between gene expression and various disease states may be determined.

The term “array” as used herein refers to any desired arrangement of a set of nucleic acids on a solid support. Specifically included within this term are so called microarrays, gene chips and the like. As used herein, the term “mitochondrial-related” coding sequence refers to those coding sequences necessary for the proper structure, assembly, and/or function of mitochondria. Such mitochondrial-related coding sequences may be found on the nuclear and mitochondrial genomes. The term “plurality of mitochondrial-related coding sequences” refers to at least 13 mitochondrial encoded genes, which represents a minimum representative sampling for screening of gene expression associated with mitochondrial structure and/or function.

Patterns of mitochondrial gene expressions in younger and older animal tissue can be screened with the invention by including in arrays nucleic acids from genes that are expressed in different tissues such including, but not limited to, liver, brain, heart, skeletal and cardiac muscle, spleen, kidney, gut, and blood. The differences in the expression of the mitochondrial genes in younger and older animals will provide insight into the regulatory processes of mtDNA in aging.

The arrays provided by the invention can also be used to study young versus aged tissues in mice, in response to a number of substances, for example, candidate drugs, inflammatory agents, heavy metals, and major acute phase reactants. The pathways associated with longevity and the effects of aging in responding to stress can thus be analyzed. The genes encoding signaling pathway intermediates activated by mitochondrial damaging agents and the genes targeting these pathways may also be examined.

The arrays provided by the invention may also be used to identify the effects of aging on liver, brain, muscle and other tissues as well as various other cells in culture; for example, to demonstrate that increased ROS due to mitochondrial damage in aged tissues may be a basic factor in the persistent activation of signals mediating chronic stress; and to demonstrate that the response to stress and injury is a major process affected by aging. Previous studies suggest that each tissue in the body could exhibit specific age-associated decrements in its ability to manifest specific response(s) to stress. The invention could thus be used to establish that responses to stress are intrinsic processes affected by aging even in the absence of disease, but whose decline can be accelerated by environmental factors and disease.

The arrays of the invention could also be used, for example, to investigate the role or effect of mitochondrial function in different diseases, including neurodegenerative diseases (Alzheimer's and Parkinson's disease), diabetes mellitus, and others (Table 1). The arrays may also be used for the development of drugs and evaluation of their effects on mitochondrial function, and for the identification and detection of modulation of mitochondrial damage in different disease states. Table 1 lists some of the Mus musculus and corresponding Homo sapiens mitochondrial genes and the human diseases associated with specific genetic defects. Accordingly, one aspect of the invention provides an array comprising nucleic acids corresponding to the accessions listed in Table 1. In one embodiment of the invention, nucleic acids of at least 5, 10, 13, 15, 20, 30 or 40 or more of the accessions given in Table 1 are included on an array of the present invention.

In another embodiment of the present invention, it is contemplated that the arrays may be used to screen “knockout” or “knockin” genes affecting mitochondrial development or function. Well known technologies such as, but not limited to, the Cre-lox system, homologous recombination, and interfering RNAs (siRNA, shRNA, RNAi) are commonly used by those skilled in the art to alter gene expression in animals or cell lines. The arrays of the present invention could be used to monitor the degree of altered gene expression which would indicate the success or failure of such experiments. For instance densitometric or fluorescent analysis of arrays of the present invention could determine the degree of expression reduction in a shRNA experiment where success or failure is measured by the degree of gene knockdown. Commonly the number of interfering RNA molecules hybridizing along a gene sequence determines the degree of expression reduction which could be compared to controls in an array experiment where one or more genes could be altered. Therefore in this embodiment the arrays of the present invention could be used to monitor one or many genes with respect to their expression levels in gene expression altering experiments.

Overall, the invention has broad applicability in that it encompasses all factors that will affect mitochondrial biogenesis and assembly (replication) and mitochondrial function under any physiological or pathophysiological conditions.

TABLE 1

| Mus musculus Gene List | | Homo sapien Gene List and Related Diseases | | |
|---|---|---|---|---|
| gene | Accession | gene | Accession | Related Disease |
| — | I48884 | — | MITOP_D1 | Deficiency of complex I |
| Abc7 | U43892 | ABAT | GABT_HUMAN | |
| Acadl | ACDL_MOUSE | ABC7 | ABC7_HUMAN | X-linked sideroblastic anemia and ataxia (XLSA/A) |
| Acadm | A55724 | ACAA2 | S43440 | |
| Acads | I49605 | ACADL | A40559 | LCAD deficiency |
| Acadvl | ACDV_MOUSE | ACADM | I52240 | MCAD deficiency |
| Acat1 | 87870 | ACADS | A30605 | SCAD deficiency |
| Acat2 | 87871 | ACADSB | A55680 | |
| Aco2 | 87880 | ACADVL | ACDB_HUMAN | VLCAD deficiency |
| Aif | AF100927 | VLCAD | | |
| Ak2 | 87978 | ACAT1 | JH0255 | Deficiency of 3-ketothiolase (3KTD) |
| Ak3 | 87979 | ACAT | | |
| Alas2 | SYMSAL | T2 | | |
| Aldh2 | I48966 | THIL | | |
| AHD-5 | | ACO2 | Q99798 | |
| AHD1 | | AFG3L2 | Y18314 | |
| AND5 | | AGXT | P21549 | |
| Ant1 | S37210 | AIF | AF100928 | |
| Ant2 | S31814 | AK2 | KAD2_HUMAN | |
| Aop1; Aop2 | JQ0064 | AK3 | KIHUA3 | |
| Atp5a1 | JC1473 | AKAP1 | I39173 | |
| Atp5b | P56480 | AKAP84 | | |
| Atp5g1 | AT91_MOUSE | AKAP84 | I39173 | |
| Atp5k | JC1412 | AKAP1 | | |
| ATP5I | | ALAS1 | SYHUAL | |
| Atp7b | U38477 | ALAS | | |
| Bax | BAXA_MOUSE | ALAS2 | SYHUAE | X-linked sideroblastic anemia (XLSA) |
| Bckdha | S71881 | ASB | | |
| Bckdhb | S39807 | ALDH2 | DEHUE2 | Alcohol intolerance, acute |
| Bcl2 | B25960 | Hs.1230 | | |
| D1Nds7 | | ALDH4 | PUT2_HUMAN | Hyperprolinemia, type II (HPII) |
| D1Nds7 | | ALDH5 | A40872 | |
| Bzrp | A53405 | AMACR | CAB44062 | Alpha-methylacyl-CoA racemase deficiency (AMACRD) |
| COII/ND5 | ND5 I76673 | AMT | I54192 | Non-ketotic hyperglycinemia, type II (NKH2) |
| Car5 | S12579 | AOP1 | TDXM_HUMAN | |
| Cbr2 | A28053 | ARG2 | ARG2_HUMAN | |
| Ckmt1 | S24612 | ATP5A1 | PWHUA | |
| Cox4 | S12142 | ATP5A2 | NNN10 | |
| Cox5a | S05495 | ATP5AL1 | NNN08 | |
| Cox5b | A39425 | ATP5AL2 | NNN09 | |
| Cox6a1 | COXD_MOUSE | ATP5B | A33370 | |
| Cox6a2 | S52088 | ATPSB | | |
| Cox6b | 107460 | ATP5BL1 | NNN06 | |
| Cox6c2 | S16083 | ATP5BL2 | NNN07 | |
| Cox7a2 | I48286 | ATP5C1 | A49108 | |
| Cox7c1 | S10303 | ATP5C2 | NNN03 | |
| Cox7c | COXO_MOUSE | ATP5CL1 | NNN04 | |
| Cox8a | COXR_MOUSE | ATP5CL2 | NNN05 | |
| Cox8b | COXQ_MOUSE | ATP5D | S22348 | |
| Cpo | A48049 | ATP5E | AF077045 | |
| Cps1 | 891996 | ATP5F1 | JQ1144 | |
| Cpt2 | A49362 | ATP5G1 | S34066 | |
| Crat | CACP_MOUSE | ATP5G2 | S34067 | |
| Cs | 88529 | ATP5G3 | I38612 | |
| Cycs | CCMS | ATP5I | AB028624 | |
| Cyct | CCMST | ATP5J | JT0563 | |
| Cyp11a | 88582 | ATP5O | ATPO_HUMAN | |
| Cyp11b1 | A41552 | OSCOP | | |
| Cyp11b2 | 88584 | ATP7B | S40525 | Wilson disease (WD) |
| Cyp24 | S60033 | BAX | BAXA_HUMAN | |
| Cyp27 | 88594 | BCAT2 | BCAM_HUMAN | |
| Dbt | S65760 | BCKDHA | DEHUXA | Maple syrup urine disease (MSUD) |
| BCKADE2 | | BCKDHB | A37157 | Maple syrup urine disease (MSUD) |
| Dci | S38770 | BCL2 | D37332 | |
| Dia1 | 94893 | BCL2L1 | BCLX_HUMAN | |
| Dld | 107450 | BCLX | | |
| Es9 | 95448 | BCS1L | AF026849 | |
| Etfa | 106092 | BDH | A42845 | |
| Etfb | 106098 | BID | BID_HUMAN | |
| Etfdh | 106100 | BNIP3L | NIPL_HUMAN | |
| Fdx1 | S53524 | BZRP-S | A49361 | |
| Fdxr | S60028 | BZRP | I38105 | |
| Fech | A37972 | C14ORF2 | 68MP_HUMAN | |
| Fpgs | S65755 | PLPM | | |
| Frda | S75712 | CA5 | CRHU5 | |
| Gcdh | GCDH_MOUSE | CACT | Y10319 | Carnitine-acylcarnitine translocase deficiency |
| Gls | 95752 | CASQ1 | A60424 | |
| Glud | S16239 | CGI-114 | T14770 | |
| Got2 | S01174 | CKMT1 | A30789 | |
| Hadh | JC4210 | CKMT2 | A35756 | |
| Hccs | CCHL_MOUSE | CLPP | S68421 | |
| Hk1 | A35244 | CLPX | CLPX_HUMAN | |
| Hmgcl | HMGL_MOUSE | COQ7 | AF032900 | |
| Hmgcs2 | B55729 | CLK-1 | | |
| Hsc70t | 96231 | COX11 | COXZ_HUMAN | |
| Hsd3b1 | I49762 | COX15 | AF044323 | |
| Hsd3b2 | 3BH2_MOUSE | COX17 | Q14061 | |
| Hsd3b3 | 3BH3_MOUSE | COX4 | OLHU4 | |
| Hsd3b4 | 3BH4_MOUSE | COX5A | OTHU5A | |
| Hsd3b5 | 3BH5_MOUSE | COX5B | OTHU5B | |
| Hsd3b6 | 3BH6_MOUSE | COX5BL4 | NNN01 | |
| Hsp60 | HHMS60 | COX6A1 | OGHU6L | |
| HSPD1 | | COX6A2 | OGHU6A | |
| Hsp70-1 | Q61698 | COX6B | OGHU6B | |
| Hsp74 | A48127 | COX6C | OGHU6C | |
| HspE1 | A55075 | COX7A1 | OSHU7A | |
| Hspe1 | CH10_MOUSE | COX7A2 | OSHU7L | |
| Idh2 | IDHP_MOUSE | COX7B | OSHU7B | |
| Maoa | I59594 | COX7C | OSHU7C | |
| Maob | 96916 | COX7RP | O14548 | |
| Mcs | A37199 | COX8 | OSHU8 | |
| Mimt44 | U69898 | CPO | I52444 | Hereditary coproporphyria (HCP) |
| Mod2 | 97045 | CPS1 | JQ1348 | Hyperammonemia, type I |
| Mor1 | DEMSMM | CPT1A | I59351 | Carnitine O-palmitoyltransferase I deficiency |
| Mthfd | A33267 | CPT1-L | | |
| Mut | S08680 | CPT1B | S70579 | |
| Ndufa4 | NUML_MOUSE | CPT2 | A39018 | Carnitine O-palmitoyltransferase II deficiency |
| Ndufs6 | NUMM_MOUSE | CPT1 | | |
| Nnt | S54876 | CRAT | A55720 | Carnitine O-acetyltransferase deficiency |
| Oat | XNMSO | CS | AF047042 | |
| Ogdh | ODO1_MOUSE | CYB5 | CBHU5 | |
| Oias1 | P11928 | CYC1 | S00680 | |
| Oias2 | P29080 | Hs.697 | | |
| Otc | OWMS | CYP11A1 | S14367 | |
| Pcca | 97499 | CYP11A | A25922 | |
| Pcx | A47255 | CYP11B1 | S11338 | Adrenal hyperplasia, type IV (AH-IV) |
| Pdha1 | S23506 | CYP11B | | |
| Pdhal | S23507 | CYP11B2 | B34181 | Deficiency of corticosterone methyloxidase, type II (CMO) |
| Pla2g2a | I48342 | CYP24 | A47436 | |
| Polg | DPOG_MOUSE | CYP27 | A39740 | Cerebrotendinous xanthomatosis (CTX) |
| Ppox | S68367 | CYP3 | A41581 | |
| Rmrp | 97937 | DBT | A32422 | Maple syrup urine disease (MSUD) |
| Rpl23 | 1196612 | DCI | A55723 | |
| Scp2 | A40015 | DECR | S53352 | Deficiency of 2,4-dienoyl-CoA reductase |
| Slc1a1 | EAT3_MOUSE | DFN1 | U66035 | Mohr-Tranebjaerg syndrome (MTS) |
| EAAC1 | | DGUOK | JC6142 | |
| Sod2 | I57023 | DHODH | PC1219 | |
| Star | A55455 | DIA1 | RDHUB5 | |
| Surf | B25394 | DLAT | XXHU | Dihydrolipoamide S-acetyltransferase deficiency; Leigh syndrome |
| Tfam | P97894 | DLTA | | |
| Tst | THTR_MOUSE | DLAT_h | S25665 | |
| Ucp | A31106 | DLD | DEHULP | Dihydrolipoamide dehydrogenase deficiency; Leigh syndrome |
| Ung | UNG_MOUSE | DLDH | | |
| UNG1 | | LAD | | |
| Vdac1 | 106919 | DLST | PN0673 | |
| Vdac2 | 106915 | DMGDH | M2GD_HUMAN | Dimethylglycine dehydrogenase deficiency (DMGDHD) |
| Vdac3 | 106922 | DUT | DUT_HUMAN | |
| Ywhaz | JC5384 | ECGF1 | P19971 | Myoneurogastrointestinal encephalopathy syndrome (MNGIE) |
| mt-Atp6 | PWMS6 | ECHS1 | ECHM_HUMAN | |
| MTATP6 | | EFE2 | TFZ_HUMAN | Barth syndrome |
| mt-Atp8 | PWMS8 | EFTS-LSB | I84606 | |
| mt-Co1 | ODMS1 | ENDOG | NUCG_HUMAN | |
| mt-Co2 | OBMS2 | ETFA | A31998 | Glutaric aciduria, type IIa (GAIIa) |
| mt-Co3 | OTMS3 | ETFB | S32482 | Glutaric aciduria, type IIb (GAIIb) |
| mt-Cytb | CBMS | ETFDH | Q16134 | Glutaric aciduria, type IIc (GAIIc) |
| COB | | FACL1 | LCFA_HUMAN | |
| mt-Nd1 | QXMS1M | FACL2 | JX0202 | |
| mt-Nd2 | QXMS2M | FARS1 | AF097441 | |
| ND2 | | FDX1 | AXHU | |
| mt-Nd3 | QXMS3M | FDX | | |
| ND3 | | FDXR | A40487 | |
| mt-Nd4 | QXMS4M | FECH | A36403 | Erythropoietic protoporphyria (EPP) |
| ND4 | | FH | UFHUM | Deficiency of fumarate hydratase |
| mt-Nd4l | QXMS4L | FPGS | A46281 | |
| mt-Nd5 | QXMS5M | FRDA1 | U43747 | Friedreich ataxia 1 |
| ND5 | | GAT | AF023466 | |
| mt-Nd6 | DEMSN6 | GATM | S41734 | |
| ND6 | | GCDH | GCDH_HUMAN | Glutaric aciduria, type I (GA-I) |
| mt-Rnr1 | 12S_rRNA | GCK | A46157 | Diabetes mellitus, type II (NIDDM) |
| mt-Rnr2 | 16S_rRNA | HK4 | | |
| mt-Ta | tAla_1 | HK4 | | |
| mt-Tc | tCys_1 | Hs.1270 | | |
| mt-Td | tAsp_1 | Hs.1270 | | |
| mt-Te | tGlu_1 | NIDDM | | |
| mt-Tf | tPhe_1 | NIDDM | | |
| mt-Tg | tGly_1 | GCSH | GCHUH | Non-ketotic hyperglycinemia, type III (NKH3) |
| mt-Th | tHis_1 | GK | GLPK_HUMAN | Glycerol kinase deficiency (GKD) |
| mt-Ti | tIle_1 | GKP2 | GKP2_HUMAN | |
| mt-Tk | tLys_1 | GLDC | B39521 | Non-ketotic hyperglycinemia, type I (NKH1) |
| mt-Tl1 | tLeu_1 | GLUD1 | DEHUE | |
| mt-Tl2 | tLeu_2 | GLUDP1 | A53719 | |
| mt-Tm | tMet_1 | GOT2 | XNHUDM | |
| mt-Tn | tAsn_1 | GPD2 | GPDM_HUMAN | Diabetes mellitus, type II (NIDDM) |
| mt-Tp | tPro_1 | GST12 | B28083 | |
| mt-Tq | tGln_1 | HADHA | JC2108 | Trifunctional enzyme deficiency; Maternal acute fatty liver of |
| | | | | pregnancy (AFLP) |
| mt-Tr | tArg_1 | HADHB | JC2109 | Trifunctional enzyme deficiency |
| mt-Ts1 | tSer_1 | HCCS | G02133 | |
| mt-Ts2 | tSer_2 | HCS | CCHU | |
| mt-Tt | tThr_1 | HHH | AF112968 | Deficiency of ornithine translocase |
| mt-Tv | tVal_1 | HIBADH | D3HI_HUMAN | |
| mt-Tw | tTrp_1 | HK1 | A31869 | |
| mt-Ty | tTyr_1 | HK2 | JC2025 | Diabetes mellitus, type II (NIDDM) |
| | | HLCS | BPL1_HUMAN | Biotin-responsive multiple carboxylase deficiency |
| | | Hs.12357 | | |
| | | HMGCL | A45470 | Hydroxymethylglutaricaciduria (HMGCL) |
| | | HMGCS2 | S51103 | |
| | | HSD3B1 | DEHUHS | Severe depletion of steroid formation |
| | | HSDB3 | | |
| | | HSD3B2 | DEHUH2 | Congenital adrenal hyperplasia (CAH) |
| | | HSPA1L | B45871 | |
| | | HSPA9 | B48127 | |
| | | GRP75 | | |
| | | HSPD1 | A32800 | |
| | | GROEL | | |
| | | HSPE1 | S47532 | |
| | | CPN10 | | |
| | | HTOM34P | Q15785 | |
| | | HTOM | AF026031 | |
| | | Hs.3816 | A56650 | |
| | | IDH2 | S57499 | |
| | | IDH3A | S55282 | |
| | | IDH3B | IDHB_HUMAN | |
| | | IDH3G | IDHG_HUMAN | |
| | | IVD | A37033 | Isovaleric acidemia (IVA) |
| | | KIAA0016 | S66619 | |
| | | TOM20 | | |
| | | KIAA0028 | SYLM_HUMAN | |
| | | KIAA0123 | Q10713 | |
| | | KNP-I | JC4913 | |
| | | LOC51081 | JC7165 | |
| | | LOC51189 | JC7175 | |
| | | LOC51629 | NP_057100 | |
| | | LOC56624 | NP_063946 | |
| | | MAOA | A36175 | Brunner's syndrome |
| | | MAOB | JH0817 | |
| | | MCD | DCMC_HUMAN | Malonyl-CoA decarboxylase deficiency (MLYCD) |
| | | MCSP | MCS_HUMAN | |
| | | MDH2 | MDHM_HUMAN | |
| | | ME2.1 | S53351 | |
| | | ME2 | A39503 | |
| | | MFT | AF283645 | |
| | | MIPEP | U80034 | |
| | | MIP | | |
| | | MLN64 | S60682 | |
| | | MMSDH | MMSA_HUMAN | Methylmalonate semialdehyde dehydrogenase deficiency |
| | | | | (MMSDHD) |
| | | MPO | OPHUM | Myeloperoxidase deficiency (MPOD) |
| | | MRRF | AA085690 | |
| | | MTRRF | | |
| | | RRF | | |
| | | MT-ACT48 | AF132950 | |
| | | MTABC3 | AF076775 | |
| | | MTATP6 | PWHU6 | Leigh syndrome; Neurogenic muscle weakness, ataxia, and retinitis |
| | | | | pigmentosa (NARP); Leber's hereditary opticneuropathy (LHON); |
| | | | | Familial bilateral striatal necrosis (FBSN) |
| | | MTATP8 | PWHU8 | |
| | | MTATT | NNN20 | |
| | | MTCH1 | AF176006 | |
| | | CHI-64 | | |
| | | MTCH2 | NP_055157 | |
| | | MTCO1 | ODHU1 | Leber's hereditary optic neuropathy (LHON); Alzheimer disease |
| | | | | (AD); Myoclonus epilepsy; deafness, ataxia, cognitive impairment |
| | | | | and Cox deficiency; Acquired idiopathic sidereoblastic |
| | | | | anemia (AISA) |
| | | MTCO2 | OBHU2 | Alzheimer disease (AD); Mitochondrial encephalomyopathies |
| | | MTCO3 | OTHU3 | Leber's hereditary optic neuropathy (LHON); Progressive |
| | | | | encephalopathy (PEM); Mitochondrial encephalomyopathies |
| | | MTCYB | CBHU | Leber's hereditary optic neuropathy (LHON); Mitochondrial |
| | | | | Myopathy (MM); Parkinsonism/MELAS overlap syndrome |
| | | COB | | |
| | | MTDLOOP | NNN21 | |
| | | MTERF | Y09615 | |
| | | MTHFD1 | A31903 | |
| | | MTHFD | | |
| | | MTHFD2 | DEHUMT | |
| | | MTHSP1 | NNN15 | |
| | | MTHSP2 | NNN16 | |
| | | MTIF2 | A55628 | |
| | | MTLSP | NNN02 | |
| | | MTND1 | DNHUN1 | Leber's hereditary optic neuropathy (LHON); Alzheimer disease |
| | | | | and Parkinson disease (ADPD); Diabetes mellitus, type II |
| | | | | (NIDDM) |
| | | MTND2 | DNHUN2 | Leber's hereditary optic neuropathy (LHON); Alzheimer |
| | | | | disease (AD) |
| | | MTND3 | DNHUN3 | |
| | | MTND4 | DNHUN4 | Leber's hereditary optic neuropathy (LHON); MELAS; Diabetes |
| | | | | mellitus, type II (NIDDM) |
| | | MTND4L | DNHUNL | Leber's hereditary optic neuropathy (LHON) |
| | | MTND5 | DNHUN5 | Leber's hereditary optic neuropathy (LHON); MELAS |
| | | MTND6 | DEHUN6 | Leber's hereditary optic neuropathy (LHON); LHON with dystonia |
| | | | | (LDYT) |
| | | MTOLR | NNN19 | |
| | | MTRF1 | RF1M_HUMAN | |
| | | MTTRF1 | | |
| | | MTRNR1 | 12s_rRNA | Aminoglycoside-induced deafness; Nonsyndromic deafness |
| | | MTRNR2 | 16S_rRNA | Chloramphenicol resistance; Alzheimer disease and Parkinson |
| | | | | disease (ADPD) |
| | | MTRNR3 | NNN17 | |
| | | MTTA | TAla | Chronic tubulointerstitial nephropathy |
| | | MTTC | TCys | Mitochondrial myopathy (MM) |
| | | MTTD | TAsp | |
| | | MTTE | TGlu | Myopathy and diabetes mellitus (MDM) |
| | | MTTER | NNN18 | |
| | | MTTF | TPhe | MELAS |
| | | MTTFH | NNN13 | |
| | | MTTFL | NNN14 | |
| | | MTTFX | NNN12 | |
| | | MTTFY | NNN11 | |
| | | MTTG | TGly | Hypertrophic cardiomyopathy; Progressive encephalopathy (PEM) |
| | | MTTH | THis | |
| | | MTTI | TIle | Fatal infantile hypertrophic cardiomyopathy (FIHC) |
| | | MTTK | TLys | MERRF; Cardiomyopathy and deafness; Myoneurogastrointestinal |
| | | | | encephalopathy syndrome (MNGIE); Diabetes mellitus-deafness |
| | | | | syndrome (DMDF) |
| | | MTTL1 | tLeu_a | MELAS; MERRF/MELAS overlap syndrome; Mitochondrial |
| | | | | myopathy (MM); Diabetes mellitus-deafness syndrome |
| | | | | (DMDF); Pediatric MMC; Adult MMC; Deafness; Maternally |
| | | | | inherited diabetes mellitus; Chronic progressive external |
| | | | | ophthalmoplegia (CPEO) |
| | | MTTL2 | tLeu_b | CPEO plus; Mitochondrial myopathy (MM) |
| | | MTTM | TMet | Mitochondrial myopathy (MM) |
| | | MTTN | TAsn | Chronic progressive external ophthalmoplegia (CPEO) |
| | | MTTP | TPro | Mitochondrial myopathy (MM) |
| | | MTTQ | TGln | Alzheimer disease and Parkinson disease (ADPD) |
| | | MTTR | TArg | |
| | | MTTS1 | tSer_1 | MERRF/MELAS overlap syndrome; Ataxia, myoclonus and |
| | | | | deafness (AMDF); Deafness; Myoclonus epilepsy, deafness, |
| | | | | ataxia, cognitive impairment and Cox deficiency; MM with RRF |
| | | MTTS2 | t_Ser2 | Diabetes mellitus-deafness syndrome (DMDF); Sensorineural |
| | | | | hearing loss and retinitis pigmentosa (DFRP) |
| | | MTTT | TThr | Lethal infantile mitochondrial myopathy (LIMM); Mitochondrial |
| | | | | myopathy (MM) |
| | | MTTV | TVal | Ataxia, progressive seizures, mental detorioration, and hearing loss |
| | | MTTW | TTrp | Dementia and chorea (DEMCHO) |
| | | MTTY | TTyr | |
| | | MTX1 | MTXN_HUMAN | |
| | | MTX2 | AAC25105 | |
| | | MUT | S40622 | Methylmalonic acidemia (MUT-, MUT0 type) |
| | | MUTYH | U63329 | |
| | | NDUFA10 | O95299 | |
| | | NDUFA1 | O15239 | |
| | | NDUFA2 | O43678 | |
| | | NDUFA3 | O95167 | |
| | | NDUFA4 | NUML_HUMAN | |
| | | NDUFA5 | NUFM_Human | |
| | | NDUFA6 | P56556 | |
| | | NDUFA7 | AAD05427 | |
| | | NDUFA8 | NUPM_HUMAN | |
| | | NDUFAB1 | T00741 | |
| | | NDUFB10 | O96000 | |
| | | NDUFB1 | O75438 | |
| | | NDUFB2 | AAD05428 | |
| | | NDUFB3 | O43676 | |
| | | NDUFB4 | O95168 | |
| | | NDUFB5 | O43674 | |
| | | NDUFB6 | O95139 | |
| | | NDUFB7 | NB8M_HUMAN | |
| | | NDUFB8 | JE0382 | |
| | | NDUFB9 | S82655 | |
| | | B22 | | |
| | | NDUFC1 | O43677 | |
| | | NDUFC2 | O95298 | |
| | | NDUFS1 | S17854 | |
| | | NDUFS2 | JE0193 | |
| | | NDUFS2L | NUEM_HUMAN | |
| | | NDUFS3 | O75489 | |
| | | NDUFS4 | NUYM_HUMAN | |
| | | NDUFS5 | O43920 | |
| | | NDUFS6 | O75380 | |
| | | NDUFS7 | O75251 | Leigh syndrome |
| | | NDUFS8 | NUIM_HUMAN | Leigh syndrome |
| | | NDUFV1 | A44362 | Alexander disease; Leigh syndrome |
| | | NDUFV2 | A30113 | |
| | | NDUFV3 | NUOM_HUMAN | |
| | | NIFS | AAD09187 | |
| | | NME4 | NDKM_HUMAN | |
| | | NNT-PEN | G02257 | |
| | | NOC4 | NP_006058 | |
| | | NRF1 | A54868 | |
| | | NTHL1 | AB001575 | |
| | | NTH1 | | |
| | | OAT | XNHUO | Ornithinemia with gyrate atrophy (GA) |
| | | OGDH | A38234 | Deficiency of alpha-ketoglutarate dehydrogenase |
| | | OGG1 | U96710 | |
| | | OIAS | A91013 | |
| | | OPA1 | T00336 | Optic atrophy (OPA1) |
| | | OTC | OWHU | Hyperammonemia, type II |
| | | OXA1L | I38079 | |
| | | OXCT | SCOT_HUMAN | Deficiency of Succinyl-CoA:3-oxoacid-CoA transferase |
| | | P43-LSB | I53499 | |
| | | P69 | A42665 | |
| | | P71 | B42665 | |
| | | PC | JC2460 | Deficiency of pyruvate carboxylase, type I and II |
| | | PCCA | A27883 | Propionic acidemia, type I (PA-1) |
| | | PCCB | A53020 | Propionic acidemia, type II (PA-2) |
| | | PCK2 | S69546 | Hypoglycemia and liver impairment |
| | | PDHA1 | DEHUPA | Pyruvate dehydrogenase deficiency; Leigh syndrome |
| | | PDHA2 | DEHUPT | |
| | | PDHB | DEHUPB | Pyruvate dehydrogenase deficiency; Leigh syndrome |
| | | PDK1 | I55465 | |
| | | PDK2 | I70159 | |
| | | PDK3 | I70160 | |
| | | PDK4 | Q16654 | |
| | | PDX1 | U82328 | Pyruvate dehydrogenase deficiency |
| | | PEMT | PEMT_HUMAN | |
| | | PEMT2 | | |
| | | PET112L | GATB_HUMAN | |
| | | PHC | A53737 | |
| | | PLA2G1B | PSHU | |
| | | PLA2 | | |
| | | PPLA2 | | |
| | | PLA2G2A | PSHUYF | |
| | | PLA2L | | |
| | | PLA2G4 | A39329 | |
| | | PLA2G5 | U03090 | |
| | | PMPCB | O75439 | |
| | | PNUTL2 | AF176379 | |
| | | POLG2 | U94703 | |
| | | POLG | G02750 | |
| | | Hs.1436 | | |
| | | POLRMT | HSU75370 | |
| | | PPOX | PPOX_HUMAN | Porphyria variegata (VP) |
| | | PRAX-1 | AF039571 | |
| | | PRDX5 | AAF03750 | |
| | | ACR1 | | |
| | | AOEB166 | | |
| | | PMP20 | | |
| | | PRXV | | |
| | | PRSS15 | S42366 | |
| | | LON-PEN | | |
| | | LON | | |
| | | PSORT | AAC05748 | |
| | | PYCR1 | A41770 | |
| | | P5C | | |
| | | RMRP | HSMRP | |
| | | RPL23L | RL23_HUMAN | |
| | | RPL23 | | |
| | | RPL3 | R5HUL3 | |
| | | RPML12 | RM12_HUMAN | |
| | | RPML19 | RLX1_HUMAN | |
| | | KIAA0104 | | |
| | | RPML37 | AAF36155 | |
| | | RPML3 | R5HUL3 | |
| | | RPMS12 | RT12_HUMAN | |
| | | SCHAD | JC4879 | |
| | | SCO2 | AL021683 | Fatal infantile cardioencephalomyopathy due to Cox deficiency |
| | | SCP2 | B40407 | |
| | | SDH1 | A34045 | |
| | | IP | | |
| | | SDH | | |
| | | SDH2 | JX0336 | Leigh syndrome; Deficiency of succinate dehydrogenase |
| | | SDHC | D49737 | Hereditary paraganglioma, type III (PGL3) |
| | | SDHD | DHSD_HUMAN | Hereditary paraganglioma, type I (PGL1) |
| | | SHMT2 | B46746 | |
| | | SLC1A1 | EAT2_HUMAN | |
| | | EAAC1 | | |
| | | SLC1A3 | JC2084 | |
| | | SLC20A3 | TXTP_HUMAN | |
| | | SLC25A12 | Y14494 | |
| | | SLC25A13 | NP_055066 | Citrullinemia, type II (CTLN2) |
| | | CTLN2 | | |
| | | SLC25A14 | O95258 | |
| | | SLC25A16 | A40141 | |
| | | GDA | | |
| | | GT | | |
| | | ML7 | | |
| | | SLC25A18 | AY008285 | |
| | | SLC25A4 | A44778 | Chronic progressive external ophthalmoplegia, type III |
| | | | | (CPEO3); Mitochondrial myopathy and cardiomyopathy (MiMyCa) |
| | | ANT1 | | |
| | | SLC25A5 | A29132 | |
| | | ANT2 | | |
| | | T3 | | |
| | | SLC25A6 | S03894 | |
| | | ANT3 | | |
| | | SLC9A6 | Q92581 | |
| | | KIAA0267 | | |
| | | SMAC | NP_063940 | |
| | | SOD2 | DSHUN | |
| | | SPG7 | Y16610 | Hereditary spastic paraplegia (HSP) |
| | | SSBP | JN0568 | |
| | | STAR | I38896 | Congenital lipoid adrenal hyperplasia |
| | | SUCLA2 | AF058953 | |
| | | SUCLG1 | P53597 | |
| | | SUCLG2 | T08812 | |
| | | SUOX | S55874 | Sulfocysteinuria |
| | | SUPV3L1 | S63453 | |
| | | SURF1 | S57749 | Leigh syndrome |
| | | SerRSmt | AB029948 | |
| | | SERS | | |
| | | mtSerRS | | |
| | | TAT | S10887 | Tyrosine transaminase deficiency, type II |
| | | | | (Richner-Hanhart syndrome) |
| | | TCF6L1 | JC1496 | |
| | | TCF6L3 | M62810 | |
| | | TFAM | X64269 | |
| | | TID1 | TID1_HUMAN | |
| | | TIM17 | IM17_HUMAN | |
| | | TIM17B | NP_005825 | |
| | | TIM23 | AF030162 | |
| | | TIM44 | IM44_HUMAN | |
| | | TK2 | KIHUT | |
| | | TPO | OPHUIT | Iodide peroxidase deficiency (IPD) |
| | | TR | THI2_HUMAN | |
| | | TR3 | | |
| | | TST | ROHU | |
| | | TUFM | S62767 | |
| | | UCP1 | A60793 | |
| | | UCP2 | UCP2_HUMAN | |
| | | UCP3 | JC5522 | |
| | | UCP4 | UCP4_HUMAN | |
| | | UNG | A60472 | |
| | | DGU | | |
| | | UDG | | |
| | | UQCRB | A32450 | |
| | | UQBP | | |
| | | UQCRC1 | A48043 | |
| | | UQCRC2 | A32629 | |
| | | UQCRFS1 | UCRI_HUMAN | Mitochondrial myopathy (MM) |
| | | UQCRH | S00219 | |
| | | UROS | A40483 | |
| | | VDAC1 | MMHUP3 | |
| | | VDAC2 | B44422 | |
| | | VDAC3 | S59547 | |
| | | VDAC4 | Q36732 | |
| | | WARS2 | AA227572 | |
| | | WFS | Y18064 | DIDMOAD |
| | | YME1L1 | AJ132637 | |
| | | YWHAE | 143E_HUMAN | |
| | | YWHAZ | PSHUAM | |

### B. The Mitochondria

1. Role of Mitochondrial Integrity in Tissue Function: Critical Factors in Mitochondrial Dysfunction and Decline in Tissue Function

It has been hypothesized that environmental factors accelerate the intrinsic processes of aging and the development of the aged phenotype. The overall results of past studies have suggested that aged tissues exhibit characteristics of chronic stress and a prolonged recovery from stress challenges. To understand the underlying basis for the development of these characteristics, the inventors have proposed that mitochondrial integrity and function may be severely affected in aged tissues due to oxidative metabolism (stress) which may lead to DNA damage and an increased production of ROS. Thus, in mitochondrial dysfunction a major factor responsible for many age-dependent changes is ROS. As a result of these homeostatic changes, there is an increase in the state of oxidative stress in aged tissues, which produces a chemical effect on the activity of signaling pathways and stress response genes. The age-associated increase of the pro-oxidant state based on continued and increased production of ROS by intrinsic and extrinsic factors enhance biological processes characteristic of chronic stress in aged tissues, and enhance development of age-associated diseases.

2. Mitochondrial Physiology

One of the primary functions of the mitochondria is the generation of cellular energy by the process of oxidative phosphorylation (OXPHOS). OXPHOS encompasses the electron transport chain (ETC) consisting of NADH dehydrogenase (complex I), succinate dehydrogenase (complex II), cytochrome c-coenzyme Q oxidoreductase (complex III) and cytochrome c oxidase (complex IV). Oxidation of NADH or succinate by the ETC generates an electrochemical gradient (Δψ) across the mitochondrial inner membrane, which is utilized by the ATP synthase (complex V) to synthesize ATP. This ATP is exchanged for cytosolic ADP by the adenine nucleotide translocator (ANT). Inhibition of the ETC results in the accumulation of electrons in the beginning of the ETC, where they can be transferred directly to O₂ to give superoxide anion (O₂—). Mitochondrial O₂— is converted to H₂O₂ by superoxide dismutase (MnSOD), and H₂O₂ is converted to H₂O by glutathione peroxidase (GPx1). The mitochondria is also the primary decision point for initiating apoptosis. This is mediated by the opening of the mitochondrial permeability transition pore (mtPTP), which couples the ANT in the inner membrane with porin (VDAC) in the outer membrane to the pro-apoptotic Bax and anti-apoptotic Bcl2. Increased mitochondrial Ca⁺⁺ or ROS and/or decreased Δψ or ATP tend to activate the mtPTP an initiate apoptosis (Wallace, 1999). Most of the above genes are components of the current microarrays.

3. The Mitochondrial Genome

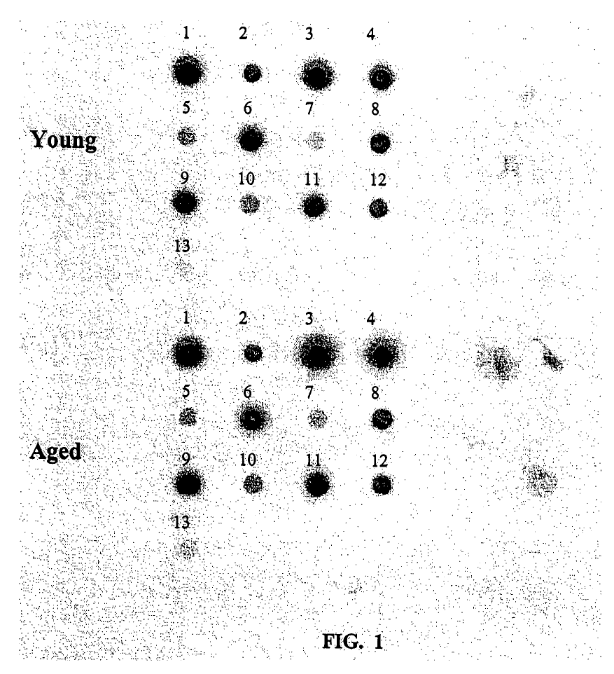

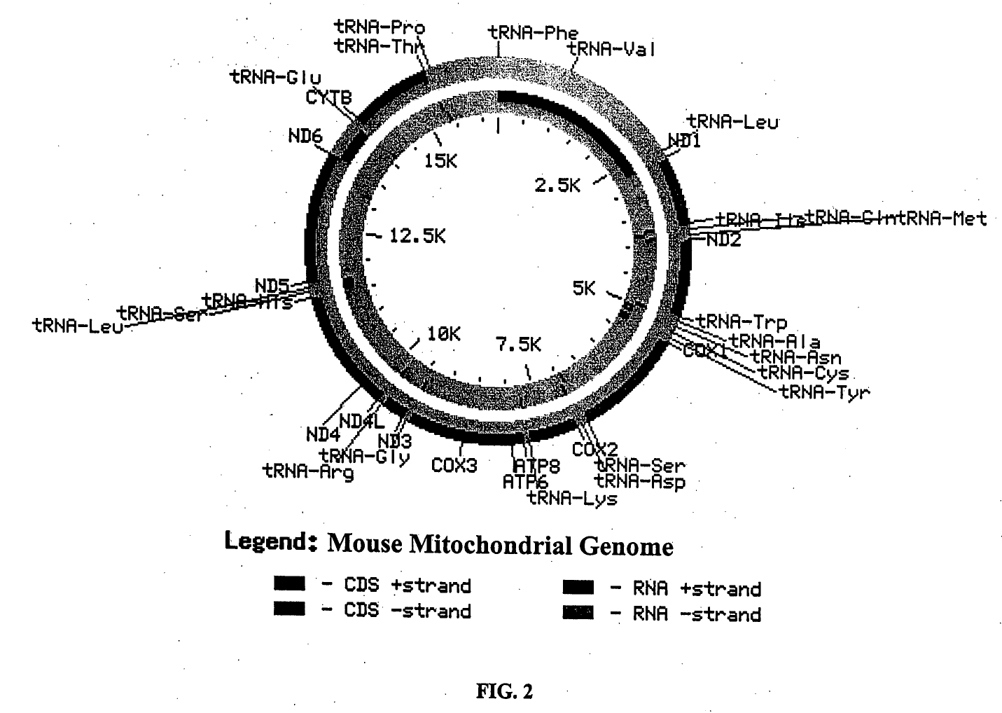

The mouse (Anderson et al., 1981) and human (Waterston et al., 2002) mitochondrial genomes consist of a single, circular double stranded DNA molecule of 16,295 and 16,569 base pairs respectively, both of which has been completely sequenced (FIGS. 1 and 2). They are present in thousands of copies in most cells and in multiple copies per mitochondrion. The mouse and human mitochondrial genomes (Tables 2-3) contain 37 genes, 28 of which are encoded on one of the strands of DNA and 9 encoded on the other. Of these genes, 24 encode RNAs (Table 3) of two types, ribosomal RNAs required for synthesis of mitochondrial proteins involved in cellular oxidative phosphorylation, and 22 amino acid carrying transfer RNAs (tRNA). The mitochondrial genome thus encodes only a small proportion of the proteins required for its specific functions; the bulk of the mitochondrial polypeptides are encoded by nuclear genes and are synthesized on cytoplasmic ribosomes before being imported into the mitochondria; examples of these genes may be found in Table 1 and on the internet on websites such as the National Center for Biotechnology Information (NCBI) website and GenomeWeb. The mitochondrial genome resembles that of a bacterium in that the genes have no introns, and that there is a very high percentage of coding DNA (about 93% of the genome is transcribed as opposed to about 3% of the nuclear genome) and a lack of repeated DNA sequences.

TABLE 2

| | | | | |
|---|---|---|---|---|
| Location | Strand | Length | Gene | Product |

Homo sapiens mitochondrion, complete genome

| | | | | |
|---|---|---|---|---|
| 3308 . . . 4264 | + | 319 | ND1 | NADH dehydrogenase subunit 1 |
| 4471 . . . 5514 | + | 348 | ND2 | NADH dehydrogenase subunit 2 |
| 5905 . . . 7446 | + | 414 | COX1 | Cytochrome c oxidase subunit I |
| 7587 . . . 8270 | + | 228 | COX2 | Cytochrome c oxidase subunit II |
| 8367 . . . 8573 | + | 69 | ATP8 | ATP synthase F0 subunit 8 |
| 8528 . . . 9208 | + | 227 | ATP6 | ATP synthase F0 subunit 6 |
| 9208 . . . 9988 | + | 260 | COX3 | Cytochrome c oxidase subunit III |
| 10060 . . . 10405 | + | 115 | ND3 | NADH dehydrogenase subunit 3 |
| 10471 . . . 10767 | + | 99 | ND4L | NADH dehydrogenase subunit 4L |
| 10761 . . . 12138 | + | 459 | ND4 | NADH dehydrogenase subunit 4 |
| 12338 . . . 14149 | + | 604 | ND5 | NADH dehydrogenase subunit 5 |
| 14150 . . . 14674 | − | 175 | ND6 | NADH dehydrogenase subunit 6 |
| 14748 . . . 15882 | + | 378 | CYTB | Cytochrome b |

Mus musculus mitochondrion, complete genome

| | | | | |
|---|---|---|---|---|
| 2760 . . . 3707 | + | 316 | ND1 | NADH dehydrogenase subunit 1 |
| 3914 . . . 4951 | + | 346 | ND2 | NADH dehydrogenase subunit 2 |
| 5328 . . . 6872 | + | 515 | COX1 | Cytochrome c oxidase subunit I |
| 7013 . . . 7696 | + | 228 | COX2 | Cytochrome c oxidase subunit II |
| 7766 . . . 7969 | + | 68 | ATP8 | ATP synthase F0 subunit 8 |
| 7927 . . . 8607 | + | 227 | ATP6 | ATP synthase F0 subunit 6 |
| 8607 . . . 9390 | + | 261 | COX3 | Cytochrome c oxidase subunit III |
| 9459 . . . 9803 | + | 115 | ND3 | NADH dehydrogenase subunit 3 |
| 9874 . . . 10167 | + | 98 | ND4L | NADH dehydrogenase subunit 4L |
| 10161 . . . 11538 | + | 459 | ND4 | NADH dehydrogenase subunit 4 |
| 11736 . . . 13559 | + | 608 | ND5 | NADH dehydrogenase subunit 5 |
| 13546 . . . 14064 | − | 173 | ND6 | NADH dehydrogenase subunit 6 |
| 14139 . . . 15282 | + | 381 | CYTB | Cytochrome b |

TABLE 3

| Mus musculus | | | Homo sapiens | | |
|---|---|---|---|---|---|
| 24 RNA Genes | | | 24 RNA Genes | | |
| Location | | Product | Location | | Product |
| Ribosomal RNAs | | | Ribosomal RNAs | | |
| 650 . . . 1603 | + | 12S ribosomal RNA | 650 . . . 1603 | + | 12S ribosomal RNA |
| 1673 . . . 3230 | + | 16S ribosomal RNA | 1673 . . . 3230 | + | 16S ribosomal RNA |
| Transfer RNAs | | | Transfer RNAs | | |
| 1 . . . 68 | + | tRNA-Phe | 579 . . . 649 | + | tRNA-Phe |
| 1025 . . . 1093 | + | tRNA-Val | 1604 . . . 1672 | + | tRNA-Val |
| 2676 . . . 2750 | + | tRNA-Leu | 3231 . . . 3305 | + | tRNA-Leu |
| 3706 . . . 3774 | + | tRNA-Ile | 4264 . . . 4332 | + | tRNA-Ile |
| 3772 . . . 3842 | − | tRNA-Gln | 4330 . . . 4401 | − | tRNA-Gln |
| 3845 . . . 3913 | + | tRNA-Met | 4403 . . . 4470 | + | tRNA-Met |
| 4950 . . . 5016 | + | tRNA-Trp | 5513 . . . 5580 | + | tRNA-Trp |
| 5018 . . . 5086 | − | tRNA-Ala | 5588 . . . 5656 | − | tRNA-Ala |
| 5089 . . . 5159 | − | tRNA-Asn | 5658 . . . 5730 | − | tRNA-Asn |
| 5192 . . . 5257 | − | tRNA-Cys | 5762 . . . 5827 | − | tRNA-Cys |
| 5260 . . . 5326 | − | tRNA-Tyr | 5827 . . . 5892 | − | tRNA-Tyr |
| 6869 . . . 6939 | − | tRNA-Ser | 7446 . . . 7517 | − | tRNA-Ser |
| 6942 . . . 7011 | + | tRNA-Asp | 7519 . . . 7586 | + | tRNA-Asp |
| 7700 . . . 7764 | + | tRNA-Lys | 8296 . . . 8365 | + | tRNA-Lys |
| 9391 . . . 9458 | + | tRNA-Gly | 9992 . . . 10059 | + | tRNA-Gly |
| 9805 . . . 9872 | + | tRNA-Arg | 10406 . . . 10470 | + | tRNA-Arg |
| 11539 . . . 11606 | + | tRNA-His | 12139 . . . 12207 | + | tRNA-His |
| 11607 . . . 11665 | + | tRNA-Ser | 12208 . . . 12266 | + | tRNA-Ser |
| 11665 . . . 11735 | + | tRNA-Leu | 12267 . . . 12337 | + | tRNA-Leu |
| 14065 . . . 14133 | − | tRNA-Glu | 14675 . . . 14743 | − | tRNA-Glu |
| 15238 . . . 15349 | + | tRNA-Thr | 15889 . . . 15954 | + | tRNA-Thr |
| 15350 . . . 15416 | − | tRNA-Pro | 15956 . . . 16024 | − | tRNA-Pro |

4. Mitochondrial DNA Mutations

Mitochondrial DNA mutations that develop during the course of a lifetime are called somatic mutations. The accumulation of somatic mutations might help explain how people who were born with mtDNA mutations often become ill after a delay of years or even decades. It is hypothesized that the buildup of random, somatic mutations depresses energy production and cause mitochondrial dysfunction that results in a decline in tissue function. This decline in the activity of proteins of the electron transport complexes involved in energy production within the mitochondria could be an important contributor to aging as well as to various age-related degenerative diseases. The characteristic hallmark of disease—a worsening over time—is thought to occur because long-term effects on certain tissues such as brain and muscle leads to progressive disease.

Other factors believed to contribute to the decline in mitochondrial energy production and its associated age-related diseases are, long-term exposure to certain environmental toxins, and accumulated somatic mutations. Mitochondria generate oxygen-free radicals that scientists believe may attack mitochondria and mutate mtDNA. Thus, somatic mutations of mtDNA contribute to the more common signs of aging (loss of strength, endurance, memory, hearing and vision) and some mtDNA mutations have been reported to increase with the age of the heart, skeletal muscle, liver, and brain regions controlling memory and motion (Melov et al., 2000). Few of these mutations can be detected before the age of 30 or 40, but they increase exponentially with age after that.

Current theories propose that progressive age-associated declines in tissue function are caused by changes in biological processes that occur in the absence of disease, and that wear and tear are major factors that accelerate this decline in tissue function. Thus, it is important to demonstrate that the development of certain intrinsic biological processes may be the basis for the gradual age-associated decline in tissue function, and ultimately for organ failure and death, and that environmental insults are important factors which may accelerate the gradual decline in tissue function. The etiologic agents that bring about homeostatic changes that occur in aged cells and tissues, include factors that generate reactive oxygen species (ROS), such as cytokines and oxidative phosphorylation. It is hypothesized that a gradual decline in tissue function is caused by the increase in the pro-oxidant state of aged tissues. Furthermore, this may be due to an elevated intrinsic oxidative stress that is mitochondrially derived, which causes an overall increase in the pro-oxidant state of aged tissues, and that such extrinsic factors as mitochondrial damaging agents intensify this pro-oxidant state. The working hypothesis states that aging increases the activity of stress factors (e.g., cytokines, ROS), and that stabilization of this new level of activity produces chronic stress in aged tissues (Papaconstantinou, 1994; Saito et al., 2001; Hsieh et al., 2002).

5. Mitochondrial Genes in Degenerative Diseases and Aging

i) Mitochondrial Diseases

It is becoming increasingly apparent that mitochondrial dysfunction is a central factor in degenerative diseases and aging. The present invention provides a tool for identifying mitochondrial genes involved in aging and age-related diseases, but is not limited to such. Mitochondrial diseases have been associated with both mtDNA and nuclear DNA (nDNA) mutations. MtDNA base substitution mutations resulting in maternally inherited diseases can affect the structure and function of proteins and protein synthesis (mutations of rRNAs and tRNAs).

In comparison with the nuclear genome, the mitochondrial genome is a small target for mutation (about 1/200,000 of the size of the nuclear genome). Thus, the proportion of clinical disease due to mutations in the mitochondrial genome might therefore be expected to be extremely low. However, due to the large amounts of non-coding DNA in the nuclear genome, most mutations in the nuclear genome do not cause diseases. In contrast, the bulk of the mitochondrial genome is composed of coding sequence and mutation rates in mitochondrial genes are thought to be about 10 times higher than those in the nuclear genome, likely because of the close proximity of the mtDNA to oxidative reactions; the number of replications is higher; and mtDNA replication is more error-prone. Accordingly, mutation in the mitochondrial genome is a significant contributor to human disease.

Mitochondrial diseases can be caused by the same types of mutations that cause disorders of the nuclear genome i.e., base substitutions, insertions, deletions and rearrangements resulting in missense or non-sense transcripts. An important aspect of the molecular pathology of mtDNA disorders, however, is whether every mtDNA molecule carries the causative mutation (homoplasmy) or whether the cell contains a mixed population of normal and mutant mitochondria (heteroplasmy). Where heteroplasmy occurs, the disease phenotype may therefore depend on the proportion of abnormal mtDNA in some critical tissue. Also, this proportion can be very different in mother and child because of the random segregation of mtDNA molecules at cell division.

The idea that defects in mitochondrial respiratory chain function might be the basis of disease has been considered for some time but it was not until 1988 that molecular analysis of mtDNA provided the first direct evidence for mtDNA mutations in neurological disorders, notably Leber's hereditary optic neuropathy. An example of a pathogenic mtDNA missense mutation is the ND6 gene mutation at nucleotide pair (np) 14459, which causes Leber's hereditary optic neuropathy (LHON) and/or dystonia. The np 14459 mutation results in a marked complex I defect, and the segregation of the heteroplasmic mutation generates the two phenotypes along the same maternal lineage (Jun et al., 1994; Jun et al., 1996).

A relatively severe mitochondrial protein synthesis disease is caused by the np 8344 mutation in the tRNALys gene resulting in myoclonic epilepsy and ragged red fiber (MERRF) disease. Mitochondrial myopathy with ragged red muscle fibers (RRFs) and abnormal mitochondria is a common feature of severe mitochondrial disease. A delayed onset and progressive course are common features of mtDNA diseases (Wallace et al., 1988; Shoffner et al., 1990). The severity as well as temporal characteristics of mtDNA mutations is illustrated by some of the most catastrophic diseases in which a the nt 4336 mutation in the tRNA^{Glu} gene is associated with late-onset Alzheimer (AD) and Parkinson Disease (PD) (Shoffner et. al., 1993).

Degenerative diseases can also be caused by rearrangements in the mtDNA. Spontaneous mtDNA deletions often present with chronic progressive external opthalmoplegia (CPEO) and mitochondrial myopathy, together with an array of other symptoms (Shoffner et. al., 1989). Maternal-inherited mtDNA rearrangement diseases are more rare.

Mitochondrial function also declines with age in the post-mitotic tissues of normal individuals. This is associated with the accumulation of somatic mtDNA rearrangement mutations in tissues such as skeletal muscle and brain (Corral-Debrinski et al., 1991; Corral-Debrinski et al., 1992a; Corral-Debrinski et al., 1992b; Corral-Debrinski et al., 1994; Horton et al., 1995; Melov et al., 1995). This same age-related accumulation of mtDNA rearrangements is seen in other multi-cellular animals including the mouse, where the accumulation of mtDNA damage is retarded by dietary restriction (Melov et al., 1997). Some examples of human disorders that can be caused by mutations in the mtDNA are listed in Table 1.

### ii) Aging and Age-Related Diseases

Several factors could cause mitochondrial energy production to decline with age even in people who start off with healthy mitochondrial and nuclear genes. Long-term exposure to certain environmental toxins is one such factor. Many of the most potent toxins known, play a role in inhibiting the mitochondria. Another factor could be the lifelong accumulation of somatic mitochondrial DNA mutations. The mitochondrial theory of aging holds that as an individual lives and produces ATP, the mitochondria generates oxygen free radicals that inexorably attack and mutate the mitochondrial DNA. This random accumulation of somatic mitochondrial DNA mutations in people who began life with healthy mitochondrial genes would ultimately reduce energy output below needed levels in one or more tissues if the individuals lived long enough. In so doing, the somatic mutations and mitochondrial inhibition could contribute to common signs of normal aging, such as loss of memory, hearing, vision, strength and stamina. In people whose energy output was already compromised (whether by inherited mitochondrial or nuclear mutations or by toxins or other factors), the resulting somatic mtDNA injury would push energy output below desirable levels more quickly. These individuals would then display symptoms earlier and would progress to full-blown disease more rapidly than would people who initially had no deficits in their energy production capacity.

There is a plethora of evidence that energy production declines and somatic mtDNA mutation increases as humans grow older. Work by many groups has shown that the activity of at least one respiratory chain complex, and possibly another, falls with age in the brain, skeletal muscle, and the heart and liver. Further, various rearrangement mutations in mtDNA have been found to increase with age in many tissues-especially in the brain (most notably in regions controlling memory and motion). Rearrangement mutations have also been shown to accumulate with age in the mtDNA of skeletal muscle, heart muscle, skin and other tissues. Certain base-substitution mutations that have been implicated in inherited mtDNA diseases may accumulate as well. All of these reports agree that few mutations reach detectable levels before age 30 or 40, but they increase exponentially after that. Studies of aging muscle attribute some of this increase to selective amplification of mitochondrial DNAs from which regions have been deleted.

### C. Arrays for Analysis of Mitochondrial-Related Gene Expression

The mitochondrial array is a complex resource that requires basic information and knowledge of procedures for constructing the genetic (DNA) sequences (components/targets) of each spot on the microarray; the preparation of DNA-probes needed to detect the mitochondrial gene products and the analysis of the resultant intensities of hybridization to the microarray chip. The arrays provided by the present invention have the potential to identify all of several hundred known mitochondrial genes identified. Further, additional genes may be added as desired and when they are identified.

The recent sequencing of the entire yeast, human, and mouse genomes has provided information on all of the mitochondrial genes of these organisms. This database has been used to search the mouse, rat and human genome databases for homologous genes. All of the known mitochondrial genes for mouse, rat and human have been identified. This information can be used for the construction of arrays for these species in accordance with the invention. In principle, DNA sequences representing all of the mitochondrial-related genes of an organism can be placed on a solid support and used as hybridization substrates to quantify the expression of the genes represented in a complex mRNA sample in accordance with the invention. Thus, the present invention provides a DNA microarray of mitochondrial and nuclear mitochondrial genes. The mitochondrial gene array will play a crucial role in the analysis of mitochondrially associated diseases, both genetic and epigenetic; it will provide the resources needed to develop drugs and pharmaceuticals to counteract such diseases; it will provide information on whether drugs affect mitochondrial function; and it will provide information on how toxic factors, hormones, growth factors, nutritional factors and stress factors affect mitochondrial function.

1. DNA Arrays

DNA array technology provides a means of rapidly screening a large number of DNA samples for their ability to hybridize to a variety of single or denatured double stranded DNA targets immobilized on a solid substrate. Techniques available include chip-based DNA technologies, such as those described by Hacia et al. (1996) and Shoemaker et al. (1996). These techniques involve quantitative methods for analyzing large numbers of genes rapidly and accurately. The technology capitalizes on the complementary binding properties of single stranded DNA to screen DNA samples by hybridization (Pease et al., 1994; Fodor et al., 1991). Basically, a DNA array consists of a solid substrate upon which an array of single or denatured double stranded DNA molecules (targets) have been immobilized.

For screening, the array may be contacted with labeled single stranded DNA probes which are allowed to hybridize under stringent conditions. The array is then scanned to determine which probes have hybridized. In a particular embodiment of the instant invention, an array would comprise targets specific for mitochondrial genes. In the context of this embodiment, such targets could include synthesized oligonucleotides, double stranded cDNA, genomic DNA, plasmid and PCR products, yeast artificial chromosomes (YACs), bacterial artificial chromosomes (BACs), chromosomal markers or other constructs a person of ordinary skill would recognize as being able to selectively hybridize to the mRNA or complements thereof of a mitochondrial-related coding sequence.

A variety of DNA array formats have been described, for example U.S. Pat. Nos. 5,861,242 and 5,578,832, which are expressly incorporated herein by reference. A means for applying the disclosed methods to the construction of such an array would be clear to one of ordinary skill in the art. In brief, in one embodiment of the invention, the basic structure of an array may comprise: (1) an excitation source; (2) an array of targets; (3) a labeled nucleic acid sample; and (4) a detector for recognizing bound nucleic acids. Such an array will typically include a suitable solid support for immobilizing the targets.

In particular embodiments of the invention, a nucleic acid probe may be tagged or labeled with a detectable label, for example, an isotope, fluorophore or any other type of label. The target nucleic acid may be immobilized onto a solid support that also supports a phototransducer and related detection circuitry. Alternatively, a gene target may be immobilized onto a membrane or filter that is then attached to a microchip or to a detector surface. In a further embodiment, the immobilized target may be tagged or labeled with a substance that emits a detectable or altered signal when combined with the nucleic acid probe. The tagged or labeled species may, for example, be fluorescent, phosphorescent, or otherwise luminescent, or it may emit Raman energy or it may absorb energy. When the probes selectively bind to a targeted species, a signal can be generated that is detected by the chip. The signal may then be processed in several ways, depending on the nature of the signal.

DNA targets may be directly or indirectly immobilized onto a solid support. The ability to directly synthesize on or attach polynucleotide probes to solid substrates is well known in the art (see U.S. Pat. Nos. 5,837,832 and 5,837,860, both of which are expressly incorporated by reference). A variety of methods have been utilized to either permanently or removably attach probes to a target/substrate (Stripping and reprobing of targets). Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, targets are stabilized and therefore may be used repeatedly. In general terms, hybridization may be performed on an immobilized nucleic acid target molecule that is attached to a solid surface such as nitrocellulose, nylon membrane or glass. Numerous other matrix materials may be used, including, but not limited to, reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules on substrates such as membranes, glass slides or beads).

Binding of probe to a selected support may be accomplished by any means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nylon membranes, the DNA probes are spotted onto the membranes. A UV light source (Stratalinker,™ Stratagene, La Jolla, Calif.) is used to irradiate DNA spots and induce cross-linking. An alternative method for cross-linking involves baking the spotted membranes at 80° C. for two hours in vacuum.

Specific DNA targets may first be immobilized onto a membrane and then attached to a membrane in contact with a transducer detection surface. This method avoids binding the target onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

2. Solid and Liquid Phase Array Assays

Genetic sequence analysis can be performed with solution and solid phase assays. These two assay formats are used individually or in combination in genetic analysis, gene expression and in infectious organism detection. Currently, genetic sequence analysis uses these two formats directly on a sample or with prepared sample DNA or RNA labeled by any one from a long list of labeling reactions. These include, 5′-Nuclease Digestion, Cleavase/Invader, Rolling Circle, and NASBA amplification systems to name a few. Epoch Biosciences has developed a powerful chemistry-based technology that can be integrated into both of these formats, using any of the amplification reactions to substantially improve their performance. These two formats include the popular homogeneous solution phase and the solid phase micro-array assays, which will be used in examples to demonstrate the technology's ability to substantially improve sensitivity and specificity of these assays.

Hybridization-based assays in modern biology require oligonucleotides that base pair (i.e., hybridize) with a nucleic acid sequence that is complementary to the oligonucleotide. Complementation is determined by the formation of specific hydrogen bonds between nucleotide bases of the two strands such that only the base pairs adenine-thymine, adenine-uracil, and guanine-cytosine form hydrogen bonds, giving sequence specificity to the double stranded duplex.

In duplex formation between an oligonucleotide and another nucleic acid molecule, the stability of the duplexes is a function of its length, number of specific (i.e., A-T, A-U, G-C) hydrogen bonded base pairs, and the base composition (ratio of G-C to A-T or A-U base pairs), since G-C base pairs provide a greater contribution to the stability of the duplex than does A-T or A-U base pairs. The quantitative measurement of a duplex's stability is expressed by its free energy (ΔG). Often a duplex's stability is measured using melting temperature (Tm)—the temperature at which one-half the duplexes have dissociated into single strands. Although ΔG is a more correct and universal measurement of duplex stability, the use of Tms in the laboratory are frequently used due to ease of measurement. Routine comparisons using Tm are an economical and sufficient way to compare this association strength characteristic, but is dependent on the nature and concentration of cations in the hybridization buffer. While many of the diagrams and charts in the site will use Tm rather than ΔG, these values were generated using constant parameters of 1×PCR buffer and 1 μm primer

Arrays in accordance with the invention may be composed of a grid of hundreds or thousands or more of individual DNA targets arranged in discrete spots on a nylon membrane or glass slide or similar support surface and may include all mitochondrial-related coding sequences that have been identified, or a selected sampling of these. A sample of single stranded nucleotide can be exposed to a support surface, and targets attached to the support surface hybridize with their complementary strands in the sample. The resulting duplexes can be detected, for example, by radioactivity, fluorescence, or similar methods, and the strength of the signal from each spot can be measured. An advantage of the arrays of the invention is that a nucleic acid sample can be probed to detect the expression levels of many genes simultaneously.

### D. Mitochondrial Nucleic Acids/Oligonucleotides

The present invention provides, in one embodiment, arrays of nucleic acid sequences immobilized on a solid support that selectively hybridize to expression products of mitochondrial-related coding sequences. Such mitochondrial-related coding sequences have been identified and include, for example, a coding sequence from the human or mouse mitochondrial genome. Sequences from the mouse mitochondrial genome are given, for example, by SEQ ID NO:1 to SEQ ID NO:13 herein.

Nucleic acids bound to a solid support may correspond to an entire coding sequence, or any other fragment thereof set forth herein. The term, “nucleic acid,” as used herein, refers to either DNA or RNA. The nucleic acid may be derived from genomic RNA as cDNA, i.e., cloned directly from the genome of mitochondria; cDNA may also be assembled from synthetic oligonucleotide segments. The nucleic acids used with the present invention may be isolated free of total viral nucleic acid.

The term “coding sequence” as used herein refers to a nucleic acid which encodes a protein or polypeptide, including a gene or cDNA. In other aspects of the invention, the term, “coding sequence” is meant to include mitochondrial genes (i.e., genes which reside in the mitochondria of a cell) as well as nuclear genes which are involved in mitochondrial structure, in mitochondrial function, or in both mitochondrial structure and mitochondrial function. Suitable genes include for example, yeast mitochondrial-related genes, C. elegans (nematode) mitochondrial-related genes, Drosophila mitochondrial-related genes, rat mitochondrial-related genes, mouse mitochondrial-related genes, and human mitochondrial-related genes. Many of the genes are known and are available at GenBank (a general database available on the internet at the National Institutes of Health website) and MitBase (see e.g., a database for mitochondrial related genes available on the internet). Other coding sequences can be readily identified by screening libraries based on homologies to known mitochondrial-related genes of other species. Some particularly suitable mitochondrial-related genes are set forth in the examples of this application.

Allowing for the degeneracy of the genetic code, sequences that have at least about 50%, usually at least about 60%, more usually about 70%, most usually about 80%, preferably at least about 90% and most preferably about 95% of nucleotides that are identical to a mitochondrial-related coding sequence may also be functionally defined as sequences that are capable of hybridizing to the mRNA or complement thereof of a mitochondrial-related coding sequence under standard conditions.

Each of the foregoing is included within all aspects of the following description. In the present invention, cDNA segments may also be used that are reverse transcribed from genomic RNA (referred to as “DNA”). As used herein, the term “oligonucleotide” refers to an RNA or DNA molecule that may be isolated free of other RNA or DNA of a particular species. “Isolated substantially away from other coding sequences” means that the sequence forms the significant part of the RNA or DNA segment and that the segment does not contain large portions of naturally-occurring coding RNA or DNA, such as large fragments or other functional genes or cDNA noncoding regions. Of course, this refers to the oligonucleotide as originally isolated, and does not exclude genes or coding regions later added to it by the hand of man.

Suitable relatively stringent hybridization conditions for selective hybridizations will be well known to those of skill in the art. The nucleic acid segments used with the present invention, regardless of the length of the sequence itself, may be combined with other RNA or DNA sequences, such that their overall length may vary considerably. It is therefore contemplated that a nucleic acid fragment of almost any length may be employed, with the total length preferably being limited by the ease of preparation and use in the intended recombinant DNA protocol.

For example, nucleic acid fragments may be prepared that include a short contiguous stretch identical to or complementary to a mitochondrial-related coding sequence, or the mRNA thereof, such as about 10-20 or about 20-30 nucleotides and that are up to about 300 nucleotides being preferred in certain cases. Other stretches of contiguous sequence that may be identical or complementary to any such sequences, including about 100, 200, 400, 800, or 1200 nucleotides, as well as the full length of the coding sequence or cDNA thereof. All that is necessary of such sequences is that selective hybridization for nucleic acids of mitochondrial-related coding sequences be carried out. The minimum length of nucleic acids capable of use in this regard will thus be known to those of skill in the art.

In principle, these oligonucleotide sequences can all selectively hybridize to a single gene such as a mitochondrial-related gene. Typically, however, the oligonucleotide sequences can be chosen such that at least one of the oligonucleotide sequences hybridizes to a first gene and at least one other of the oligonucleotide sequences hybridizes to a second, different gene.

As indicated above, the array can include a plurality of oligonucleotide sequences. For example, the array can include at least 5 oligonucleotide sequences, and each of the 5 oligonucleotide sequences can selectively hybridize to genes. In this case, a first oligonucleotide sequence would selectively hybridize to a first gene; a second oligonucleotide sequence would selectively hybridize to a second gene; a third oligonucleotide sequence would selectively hybridize to a third gene; a fourth oligonucleotide sequence would selectively hybridize to a fourth gene; and a fifth oligonucleotide sequence would selectively hybridize to a fifth gene, and each of the first, second, third, fourth and fifth genes would be different from one another.

1. Oligonucleotide Probes and Primers

The various probes and targets used with the present invention may be of any suitable length. Naturally, the present invention encompasses use of RNA and DNA segments that are complementary, or essentially complementary, to a mitochondrial-related coding sequence. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a mitochondrial-related coding sequence, including the mRNA and cDNA thereof, under relatively stringent conditions such as those described herein. Such sequences may encode the entire sequence of the mitochondrial coding sequence or fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. Oligonucleotide targets may also be attached to substrates such that each target selectively hybridizes to a separate region along a single gene for the purposes of identification and detection of gene mutations including, rearrangements, deletions, insertions, or single nucleotide polymorphisms (SNP) based on reduced probe signal compared to normal control signals.

### E. Assaying for Relative Expression of Mitochondrial-Related Coding Sequences

The present invention, in various embodiments, involves assaying for gene expression. There are a wide variety of methods for assessing gene expression, most which are reliant on hybridization analysis. In specific embodiments, template-based amplification methods are used to generate (quantitatively) detectable amounts of gene products, which are assessed in various manners. The following techniques and reagents will be useful in accordance with the present invention.

Nucleic acids used for screening may be isolated from cells contained in a biological sample, according to standard methodologies (Sambrook et al., 1989 and 2001). The nucleic acid may be genomic DNA or RNA or fractionated or whole cell RNA. Where RNA is used, it may be desired to convert the RNA to a complementary DNA using reverse transcriptase (RT). In one embodiment, the RNA is mRNA and is used directly as the template for probe construction. In others, mRNA is first converted to a complementary DNA sequence (cDNA) and this product is amplified according to protocols described below.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

The phrase, “selectively hybridizing to” refers to a nucleic acid that hybridizes, duplexes, or binds only to a particular target DNA or RNA sequence when the target sequences are present in a preparation of DNA or RNA. By selectively hybridizing, it is meant that a nucleic acid molecule binds to a given target in a manner that is detectable in a different manner from non-target sequence under moderate, or more preferably under high, stringency conditions of hybridization. Proper annealing conditions depend, for example, upon a nucleic acid molecule's length, base composition, and the number of mismatches and their position on the molecule, and must often be determined empirically. For discussions of nucleic acid molecule (probe) design and annealing conditions, see, for example, Sambrook et al., (1989 and 2001).

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

High stringency hybridization conditions are selected at about 5° C. lower than the thermal melting point—Tm—for the specific sequence at a defined ionic strength and pH. The Tm is the temperature (under defined ionic strength and pH) at which 50% of the target sequence hybridizes to a perfectly matched probe. As other factors may significantly affect the stringency of hybridization, including, among others, base composition and size of complementary strands, the presence of organic solvents, i.e., salt or formamide concentration, and the extent of base mismatching, the combination of parameters is more important than the absolute measure of any one. High stringency may be attained, for example, by overnight hybridization at about 68° C. in a 6×SSC solution, washing at room temperature with a 6×SSC solution, followed by washing at about 68° C. in a 6×SSC solution then in a 0.6×SSX solution or using commercially available proprietary hybridization solutions such as that offered by ClonTech™.

Hybridization with moderate stringency may be attained, for example, by: (1) filter pre-hybridizing and hybridizing with a solution of 3× sodium chloride, sodium citrate (SSC), 50% formamide, 0.1M Tris buffer at pH 7.5, 5× Denhart's solution; (2) pre-hybridization at 37° C. for 4 hours; (3) hybridization at 37° C. with amount of labeled probe equal to 3,000,000 cpm total for 16 hours; (4) wash in 2×SSC and 0.1% SDS solution; (5) wash 4× for 1 minute each at room temperature and 4× for 30 minutes each; and (6) dry and expose to film.

It is also understood that the ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

Generally, nucleic acid sequences suitable for use in the arrays of the present invention (i.e., those oligonucleotide sequences that selectively hybridize to mitochondrial-related genes) can be identified by comparing portions of a mitochondrial-related gene's sequence to other known sequences (e.g., to the other sequences described in GenBank) until a portion that is unique to the mitochondrial-related gene is identified. This can be done using conventional methods and is preferably carried out with the aid of a computer program, such as the BLAST program. Once such a unique portion of the mitochondrial-related gene is identified, flanking primers can be prepared and targets corresponding to the unique portion can be produced using, for example, conventional PCR techniques. This method of identification, preparation of flanking primers, and preparation of oligonucleotides is repeated for each of the mitochondrial-related genes of interest.

Once the oligonucleotide target sequences corresponding to the mitochondrial-related genes of interest are prepared, they can be used to make an array. Arrays can be made by immobilizing (e.g., covalently binding) each of the nucleic acids targets at a specific, localized, and different region of a solid support. As described herein, these arrays can be used to determine the expression of one or more mitochondrial-related genes in a cell line, in a tissue or tissues of interest. The method may involve contacting the array with a sample of material from cells or tissues under conditions effective for the expression products of mitochondrial-related genes to hybridize to the immobilized oligonucleotide target sequences. Illustratively, isostopic or fluorometric detection can be effected by labeling the material from cells or tissue with a radioisotope which will be incorporated into the probe during or after reverse transcriptase (RT) reaction or fluorescent labeled nucleotide (A,T,C,G,U) (e.g., flourescein), washing non-hybridized material from the array after hybridization is permitted to take place, and detecting whether a (labeled) mitochondrial-related gene transcripts hybridized to a particular target using, for example, phosphorimagers or laser scanners for detection of label and the knowledge of where in the array the particular oligonucleotide was immobilized. The arrays of the present invention can be used for a variety of other applications related to mitochondrial structure, function, and mutations as described herein.

### F. Screening For Modulators of Mitochondrial Function

The present invention further comprises methods for identifying modulators of the mitochondrial structure and/or function. These assays may comprise random screening of large libraries of candidate substances; alternatively, the assays may be used to focus on particular classes of compounds selected with an eye towards structural attributes that are believed to make them more likely to modulate the function or expression of mitochondrial genes.

To identify a modulator, one generally may determine the expression or activity of a mitochondrial gene in the presence and absence of the candidate substance, a modulator defined as any substance that alters function or expression. Assays may be conducted in cell free systems, in isolated cells, or in organisms including transgenic animals. It will, of course, be understood that all the screening methods of the present invention are useful in themselves notwithstanding the fact that effective candidates may not be found. The invention provides methods for screening for such candidates, not solely methods of finding them.

As used herein, the term “candidate substance” refers to any molecule that may potentially inhibit or enhance activity or expression of a mitochondrial or mitochondrial related gene. The candidate substance may be a protein or fragment thereof, a small molecule, a nucleic acid molecule or expression construct. It may be that the most useful pharmacological compounds will be compounds that are structurally related to a mitochondrial gene or a binding partner or substrate therefore. Using lead compounds to help develop improved compounds is know as “rational drug design” and includes not only comparisons with known inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fingi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include RNA interference molecules, antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be an ideal candidate inhibitor.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

### G. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Capability and Feasibility Studies

In order to demonstrate the capability of the present invention, a DNA microarray was generated from PCR products using thirteen genes that code for the mitochondrial proteins (FIG. 1). These genes were attached to nylon membranes by cross linking with UV radiation.

Positions #1 to #13 on array 1 (young) and array 2 (aged) contain the 13 mitochondrial gene targets. A hybridization study was carried out using samples from young vs aged mouse livers. The samples were labeled by reverse transcriptase incorporation of radiolabeled nucleotides and the results were observed by autoradiography. Intense and specific hybridization signals were detected at all positions indicating levels of transcript abundance.

The data showed a successful hybridization of a limited set of mitochondrial genes on the test array.

### Example 2

### Location of Mus Musculus and Homo sapiens Mitochondrial Peptides and Proteins

FIGS. 2 and 3, are maps of the human and mouse (Mus musculus) mitochondrial genomes which show the location of the 13 peptides of the OXPHOS complexes, 22 tRNAs, and 2 rRNAs that are encoded by the mitochondrial genome, and that were used, in part, to prepare an array of the present invention.

Table 2 shows the location of the Mus Musculus and Homo sapien mitochondrial proteins (13 polypeptides). It gives their location (nucleotides), strand, length of polypeptide (number of amino acids) name of the gene, and the protein products which was used in part as targets for an array of the present invention. Table 3 shows the location of the Mus musculus and Homo sapiens mitochondrial 12S and 16S ribosomal RNAs and 22 tRNA.

### Example 3

### Effects of Rotenone on Expression of Mouse Mitochondria Genes

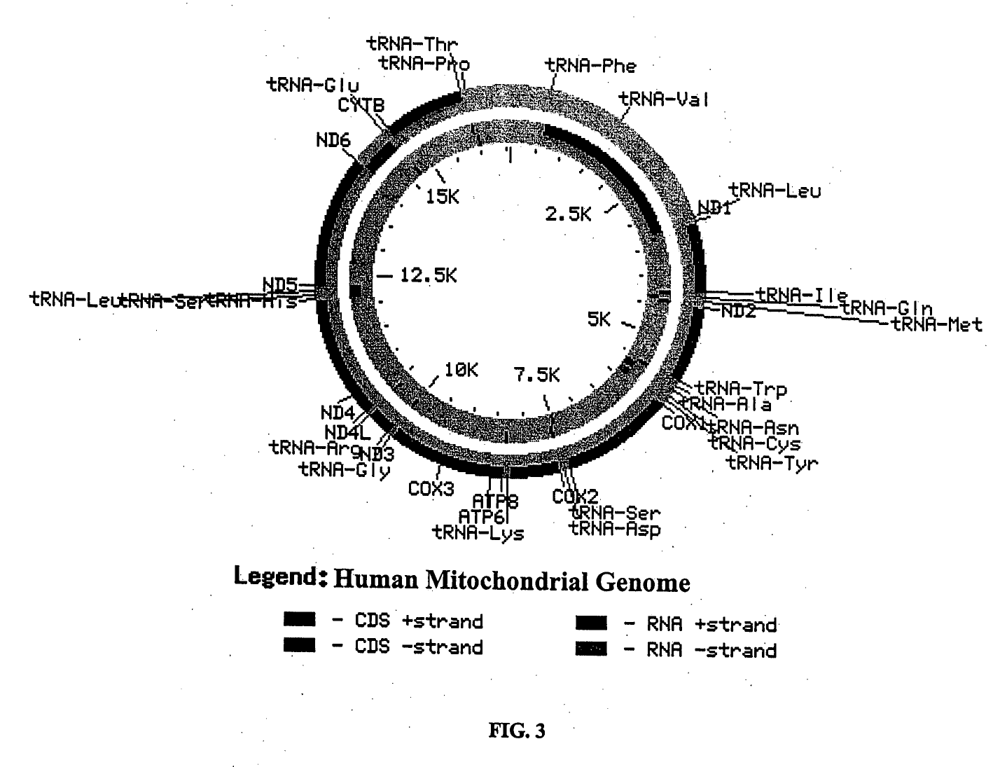

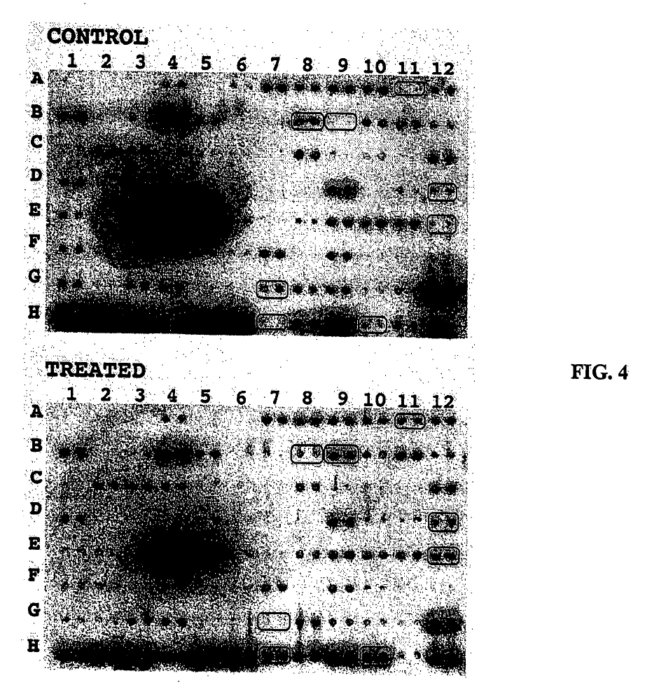

The effects of rotenone, an inhibitor of mitochondrial Complex I, on the expression of mouse mitochondrial genes in AML-12 mouse liver cells in culture were examined (FIG. 4; Table 4). The microarrays show the mRNAs whose pool levels are up-regulated. Spots A1-G11 represent mitochondrial related nuclear encoded genes; spots G12-H12 represent the 13 genes encoded by mitochondrial DNA. It should be noted that in subsequent microarray designs (constructions) the mitochondrial DNA encoded genes G12-H12 were removed from the filters and arrayed separately. Thus, the G12-H12 spots were replaced with nuclear encoded genes. The following data suggest that the a number of genes are up-regulated in response to rotenone treatment: A11, ATP synthase lipid binding proteins; B8, ADP, ATP carrier protein; B9, cytochrome C oxidase chain VIIa; D12, chaperonin 10; E12, pyruvate carboxylase; H7, Complex I: Protein Dehydrogenase chain 3. E4 and E5 represent the 23S and 16S mitochondrial ribosomal RNAs. The data also suggest that inhibition of Complex I may stimulate the production of mRNAs of Complex I proteins (H7, H10), suggesting a compensatory response to the inhibitor.

TABLE 4
Micro array template for FIG. 4

| | | | | | | | | | | | | |
|---|---|---|---|---|---|---|---|---|---|---|---|---|
| A | 1 | 2 | 3 | 4 | 5 | 6 | 7 | 8 | 9 | 10 | 11 | 12 |
| B | 13 | 14 | 15 | 16 | 17 | 18 | 19 | 20 | 21 | 22 | 23 | 24 |
| C | 25 | 26 | 27 | 28 | 29 | 30 | 31 | 32 | 33 | 34 | 35 | 36 |
| D | 37 | 38 | 39 | 40 | 41 | 42 | 43 | 44 | 45 | 46 | 47 | 48 |
| E | 49 | 50 | 51 | 52 | 53 | 54 | 55 | 56 | 57 | 58 | 59 | 60 |
| F | 61 | 62 | 63 | 64 | 65 | 66 | 67 | 68 | 69 | 70 | 71 | 72 |
| G | 73 | 74 | 75 | 76 | 77 | 78 | 79 | 80 | 81 | 82 | 83 | 84 |
| H | 85 | 86 | 87 | 88 | 89 | 90 | 91 | 92 | 93 | 94 | 95 | 96 |

| real | | | | |
|---|---|---|---|---|
| number | | | | |
| PCR | spot # | Gene name | Mitop/genbank | Description 10 ng/spot, 0.1 μM each primer |
| 1 | 1 | Acadl | ACDL_MOUSE | Acyl-CoA dehydrogenase, long-chain specific |
| | | | | precursor (LCAD) |
| 2 | 2 | Acadm | A55724 | Acyl-CoA dehydrogenase, medium-chain specific |
| | | | | precursor (MCAD) |
| 3 | 3 | Acads | I49605 | Acyl-CoA dehydrogenase, short-chain specific |
| | | | | precursor |
| 4 | 4 | Aif | AF100927 | Apoptosis-inducing factor |
| 5 | 5 | Alas2 | SYMSAL | 5-aminolevulinate synthase precursor |
| 6 | 6 | Aldh2 | I48966 | Aldehyde dehydrogenase (NAD+) 2 precursor |
| 7 | 7 | Ant1 | S37210 | ADP, ATP carrier protein, heart isoform T1 |
| 8 | 8 | Ant2 | S31814 | ADP, ATP carrier protein, fibroblast isoform 2 |
| 9 | 9 | Aop1; Aop2 | JQ0064 | MER5 protein |
| 10 | 10 | Atp5a1 | JC1473 | H+-transporting ATP synthase chain alpha |
| 11 | 11 | Atp5g1 | ATPL_MOUSE | ATP synthase lipid-binding protein P1 precursor |
| | | | | (protein 9) |
| 12 | 12 | Atp7b | U38477 | Probable copper transporting P-type ATPase |
| 13 | 13 | Bax | BAXA_MOUSE | Apoptosis regulator BAX, membrane isoform alpha |
| 14 | 14 | Bckdha | S71881 | Branched chain alpha-ketoacid dehydrogenase |
| | | | | chain E1-alpha |
| 15 | 15 | Bckdhb | S39807 | 3-methyl-2-oxobutanoate dehydrogenase |
| | | | | (lipoamide) |
| 16 | 16 | Bcl2 | B25960 | Transforming protein bcl-2-beta |
| 17 | 17 | Bzrp | A53405 | Peripheral-type benzodiazepine receptor 1 |
| 18 | 18 | Car5 | S12579 | Carbonate dehydratase, hepatic |
| 20 | 19 | Ckmt1 | S24612 | Creatine kinase |
| 21 | 20 | Cox4 | S12142 | Cytochrome c oxidase chain IV precursor |
| 23 | 21 | Cox7a2 | I48286 | Cytochrome C oxydase polypeptide VIIa- |
| | | | | liver/heart precursor |
| 24 | 22 | Cox8a | COXR_MOUSE | Cytochrome c oxidase chain VIII |
| 25 | 23 | Cpo | A48049 | Coproporphyrinogen oxidase |
| 26 | 24 | Cpt2 | A49362 | Carnitine O-palmitoyltransferase II precursor |
| 27 | 25 | Crat | CACP_MOUSE | Carnitine O-acetyltransferase (carnitine acetylase) |
| 28 | 26 | Cycs | CCMS | Cytochrome C, somatic |
| 31 | 27 | Dbt | S65760 | Dihydrolipoamide transacylase precursor |
| 32 | 28 | Dci | S38770 | 3,2-trans-enoyl-CoA isomerase, mitochondrial |
| | | | | precursor |
| 33 | 29 | Dld | 107450 | Dihydrolipoamide dehydrogenase (E3) |
| 34 | 30 | Fdx1 | S53524 | Adrenodoxin precursor |
| 35 | 31 | Fdxr | S60028 | Ferredoxin--NADP+ reductase precursor |
| 124 | 32 | Nrf1 | NM_010938 | Nuclear respiratory factor |
| 37 | 33 | Fpgs | S65755 | Tetrahydrofolylpolyglutamate synthase precursor |
| 38 | 34 | Frda | S75712 | Friedreich ataxia |
| 39 | 35 | Gcdh | GCDH_MOUSE | Glutaryl-CoA dehydrogenase precursor (GCD) |
| 40 | 36 | Glud | S16239 | Glutamate dehydrogenase (NAD(P)+) precursor |
| 41 | 37 | Got2 | S01174 | Glutamate oxaloacetaate transaminase-2 |
| 42 | 38 | Hadh | JC4210 | 3-hydroxyacyl-CoA dehydrogenase, short chain- |
| | | | | specific, precursor |
| 43 | 39 | Hccs | CCHLMOUSE | Cytochrome C-type heme lyase (CCHL) |
| 44 | 40 | Hk1 | A35244 | Hexokinase I |
| 45 | 41 | Hmgc1 | HMGL_MOUSE | Hydroxymethylglutaryl-CoA lyase |
| 46 | 42 | Hmgcs2 | B55729 | Hydroxymethylglutaryl-CoA synthase, |
| | | | | mitochondrial |
| 47 | 43 | Hsc70t | 96231 | Heat shock protein cognate 70, testis |
| 48 | 44 | Hsd3b1 | 3BH1_MOUSE | 3-beta hydroxy-5-ene steroid dehydrogenase type I |
| 49 | 45 | Hsp60 | HHMS60 | Heat shock protein 60 precursor |
| 50 | 46 | Hsp70-1 | Q61698 | Heat shock protein, 70K (hsp68) (fragment) |
| Blank | 47 | Blank | Blank | |
| 52 | 48 | HspE1 | A55075 | Chaperonin-10 |
| 53 | 49 | Idh2 | IDHP_MOUSE | Isocitrate dehydrogenase (NADP) |
| 54 | 50 | Mimt44 | U69898 | TIM44-mitochondrial inner membrane import |
| | | | | subunit |
| 55 | 51 | Mor1 | DEMSMM | Malate dehydrogenase precursor, mitochondrial |
| 56 | 52 | mt-Rnr1 | 12S_rRNA | 12S rRNA |
| 57 | 53 | mt-Rnr2 | 16S_rRNA | 16S rRNA |
| 58 | 54 | Mthfd | A33267 | Methylenetetrahydrofolate dehydrogenase (NAD+) |
| 59 | 55 | Mut | S08680 | Methylmalonyl-CoA mutase alpha chain precursor |
| 60 | 56 | Nnt | S54876 | NAD(P)+ transhydrogenase (B-specific) precursor |
| 61 | 57 | Oat | XNMSO | Ornithine--oxo-acid transaminase precursor |
| 62 | 58 | Oias1 | 25A1_MOUSE | (2′-5′)oligoadenylate synthetase 1 |
| 64 | 59 | Otc | OWMS | Ornithine carbamoyltransferase precuresor |
| 65 | 60 | Pcx | A47255 | Pyruvate carboxylase |
| 66 | 61 | Pdha1 | S23506 | Pyruvate dehydrogenase (lipoamide) |
| 67 | 62 | Pdha1 | S23507 | Pyruvate dehydrogenase (lipoamide) |
| 69 | 63 | Polg | DPOG_MOUSE | DNA polymerase gamma |
| 70 | 64 | Ppox | S68367 | Protoporphyrinogen oxidase |
| 71 | 65 | Rpl23 | 1196612 | L23 mitochondrial-related protein |
| 72 | 66 | Scp2 | JU0157 | Sterol carrier protein x |
| 74 | 67 | Sod2 | I57023 | Superoxide dismutase (Mn) precursor |
| 75 | 68 | Star | A55455 | Steroidogenic acute regulatory protein precursor, |
| | | | | mitochondrial |
| 76 | 69 | Tfam | P97894 | Mitochondrial transcription factor A - mouse |
| 77 | 70 | Tst | THTR_MOUSE | Thiosulfate sulfurtransferase |
| 79 | 71 | Ung | UNG_MOUSE | Uracil-DNA glycosylase |
| 80 | 72 | Vdac1 | 106919 | Voltage-dependent anion channel 1 |
| 81 | 73 | Vdac2 | 106915 | Voltage-dependent anion channel 2 |
| 82 | 74 | Vdac3 | 106922 | Voltage-dependent anion channel 3 |
| 83 | 75 | Ywhaz | JC5384 | 14-3-3 protein zeta/delta |
| 84(non- | 76 | WS-3 | | |
| Mitop) | | | | |
| 85(non- | 77 | Skd3 | | |
| Mitop) | | | | |
| 93(non- | 78 | | L00923 | Myosin 1 |
| Mitop) | | | | |
| 94 | 79 | GAPDH | M32599 | Glyceraldehyde 3-phosphate dehydrogenase |
| | | | | (G3PDH) |
| 108(non- | 80 | Hsd3b5 | L41519 | 3-keytosteroid reductase |
| Mitop) | | | | |
| 119 | 81 | APE 1 | P28352 | Apurinic/apyrimidinic endonuclease 1 |
| 122 | 82 | Ogdh | U02971 | 2-Oxoglutarate dehydrogenase E1 component |
| 123 | 83 | ACADV | U41497 | Acyl-Co A dehydrogenase very long chain |
| Mito13 | 84 | mt-Nd1 | QXMS1M | Protein 1 (NADH dehydrogenase (ubiquinone) |
| 95 | | | | chain 1) |
| Mito13 | 85 | mt-Nd2 | QXMS2M | Protein 2 (NADH dehydrogenase (ubiquinone) |
| 96 | | | | chain 2) |
| Mito13 | 86 | mt-Co1 | ODMS1 | Cytochrome c oxidase subunit I |
| 97 | | | | |
| Mito13 | 87 | mt-Co2 | OBMS2 | Cytochrome c oxidase subunit II |
| 98 | | | | |
| Mito13 | 88 | mt-Atp8 | PWMS8 | Protein A61 (H+-transporting ATP synthase protein |
| 99 | | | | 8) |
| Mito13 | 89 | mt-Atp6 | PWMS6 | ATPase 6 (H+-transporting ATP synthase protein |
| 100 | | | | 6) |
| Mito13 | 90 | mt-Co3 | OTMS3 | Cytochrome c oxidase subunit III |
| 101 | | | | |
| Mito13 | 91 | mt-Nd3 | QXMS3M | Protein 3 (NADH dehydrogenase (ubiquinone) |
| 102 | | | | chain 3) |
| Mito13 | 92 | mt-Nd41 | QXMS4L | Protein 4L (NADH dehydrogenase (ubiquinone) |
| 103 | | | | chain 4L) |
| Mito13 | 93 | mt-Nd4 | QXMS4M | Protein 4 (NADH dehydrogenase (ubiquinone) |
| 104 | | | | chain 4) |
| Mito13 | 94 | mt-Nd5 | QXMS5M | Protein 5 (NADH dehydrogenase (ubiquinone) |
| 105 | | | | chain 5) |
| Mito13 | 95 | mt-Nd6 | DEMSN6 | Protein 6 (NADH dehydrogenase (ubiquinone) |
| 106 | | | | chain 6 |
| Mito13 | 96 | mt-Cytb | CBMS | Cytochrome b (ubiquinol--cytochrome c reductase |
| 107 | | | | subunit III) |

### Example 4

### Effects of 3-Nitropropionic Acid and Trypanosome Infection on Expression of Mitochondrial Genes

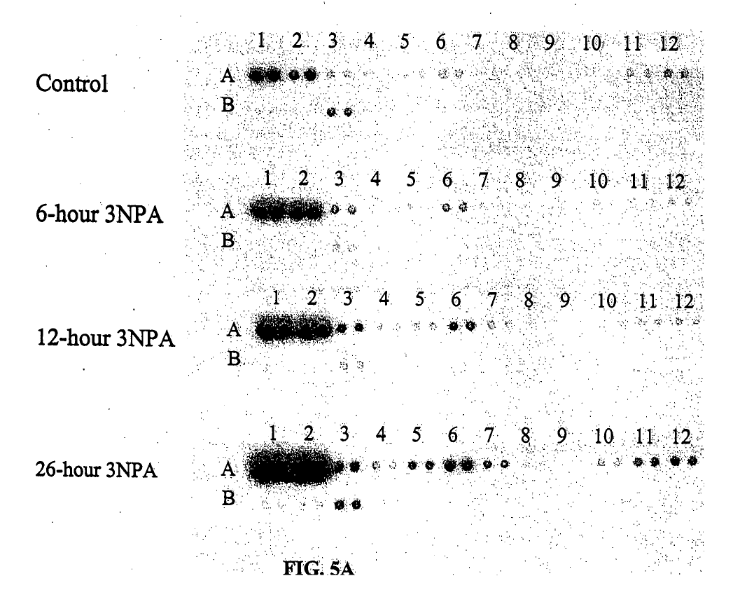

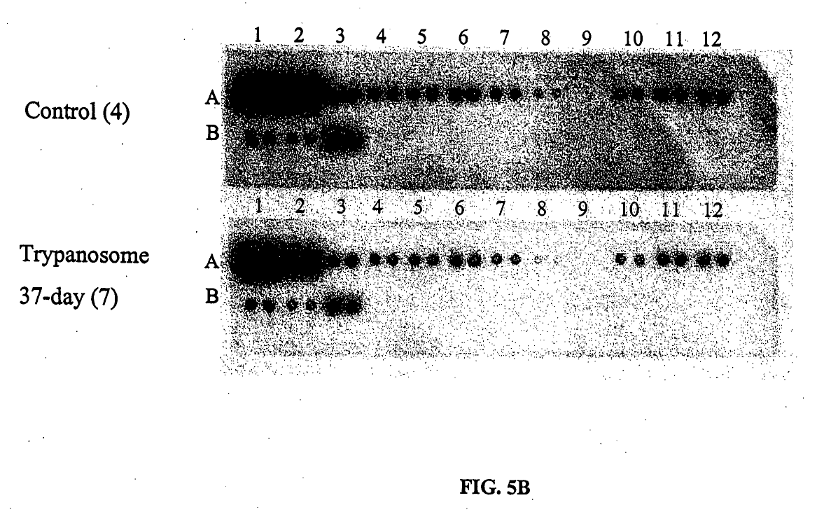

Analysis of mitochondrial DNA encoded gene expression in response to 3-nitropropionic acid (3NPA), an inhibitor of Complex II—succinic dehydrogenase was performed (FIG. 5A, Table 5). The 3 NPA treatments were at 6, 12 and 26 hours. The data showed that inhibition of Complex II stimulates the synthesis of mitochondrial encoded mRNAs and the 23S and 16S ribosomal RNAs.

In an example of overall gene down-regulation an analysis of mitochondrial DNA encoded gene expression in trypanosome infected heart tissue was also performed (FIG. 5B, Table 5). These data showed a decline in mRNA and ribosomal RNA levels at 37 days post infection.

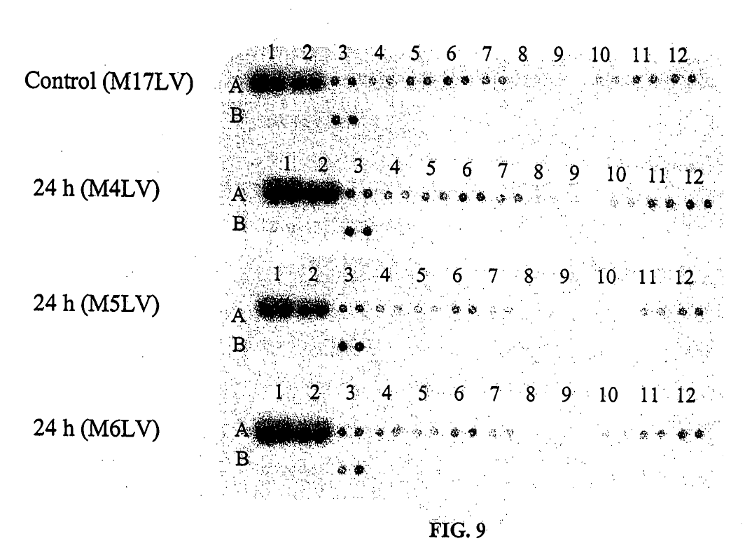

TABLE 5
Microarray template for FIGS. 5A, 5B and 9

| | | | | | | | | | | | | |
|---|---|---|---|---|---|---|---|---|---|---|---|---|
| A | 1 | 2 | 3 | 4 | 5 | 6 | 7 | 8 | 9 | 10 | 11 | 12 |
| B | 13 | 14 | 15 | 16 | 17 | | | | | | | |

| | | | |
|---|---|---|---|
| 1 | mt-Rnr1 | 12S_rRNA | 12S rRNA |
| 2 | mt-Rnr2 | 16S_rRNA | 16S rRNA |
| 3 | mt-Nd1 | QXMS1M | Protein 1 (NADH dehydrogenase |
| | | | (ubiquinone) chain 1) |
| 4 | mt-Nd2 | QXMS2M | Protein 2 (NADH dehydrogenase |
| | | | (ubiquinone) chain 2) |
| 5 | mt-Co1 | ODMS1 | cytochrome c oxidase subunit I |
| 6 | mt-Co2 | OBMS2 | cytochrome c oxidase subunit II |
| 7 | mt-Atp8 | PWMS8 | Protein A61 (H+-transporting ATP |
| | | | synthase protein 8) |
| 8 | mt-Atp6 | PWMS6 | ATPase 6 (H+-transporting ATP |
| | | | synthase protein 6) |
| 9 | mt-Co3 | OTMS3 | cytochrome c oxidase subunit III |
| 10 | mt-Nd3 | QXMS3M | Protein 3 (NADH dehydrogenase |
| | | | (ubiquinone) chain 3) |
| 11 | mt-Nd4l | QXMS4L | Protein 4L (NADH dehydrogenase |
| | | | (ubiquinone) chain 4L) |
| 12 | mt-Nd4 | QXMS4M | Protein 4 (NADH dehydrogenase |
| | | | (ubiquinone) chain 4) |
| 13 | mt-Nd5 | QXMS5M | Protein 5 (NADH dehydrogenase |
| | | | (ubiquinone) chain 5) |
| 14 | mt-Nd6 | DEMSN6 | Protein 6 (NADH dehydrogenase |
| | | | (ubiquinone) chain 6 |
| 15 | mt-Cytb | CBMS | Cytochrome b (ubiquinol--cytochrome c |
| | | | reductase subunit III) |
| 16 | GAPDH | M32599 | Glyceraldehyde 3-phosphate |
| | | | dehydrogenase (G3PDH) |
| 17 | β-actin | X03672 | beta-actin |

### Example 5

### Mitochondrial Gene Expression In Livers of Young and Aged Snell Dwarf Mouse Mutants

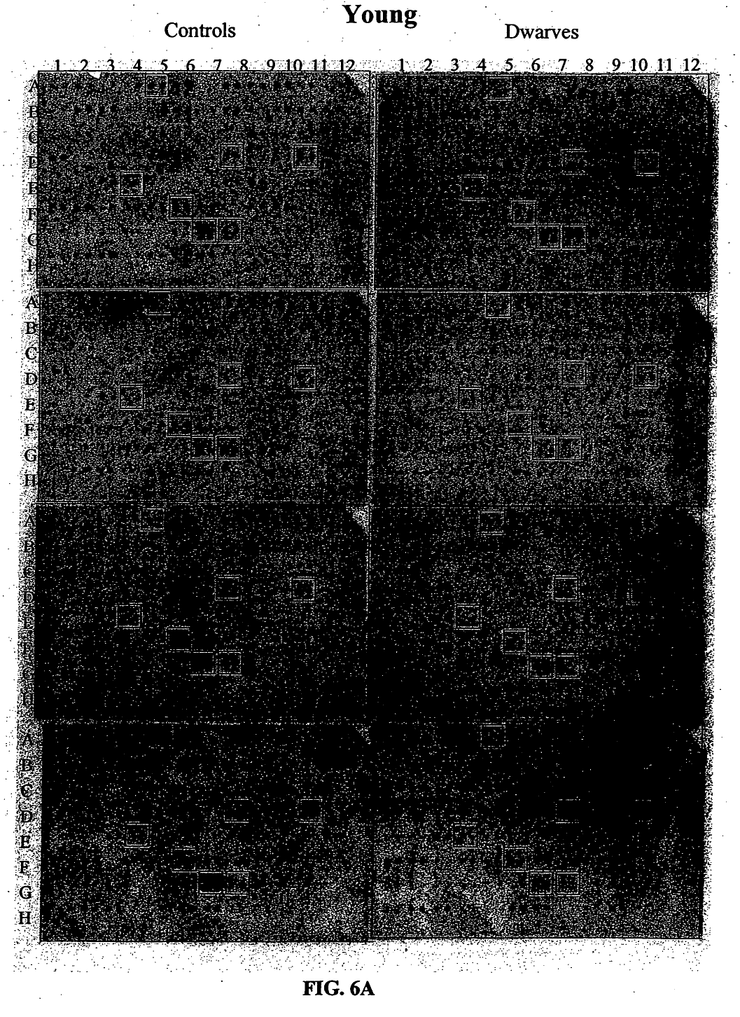

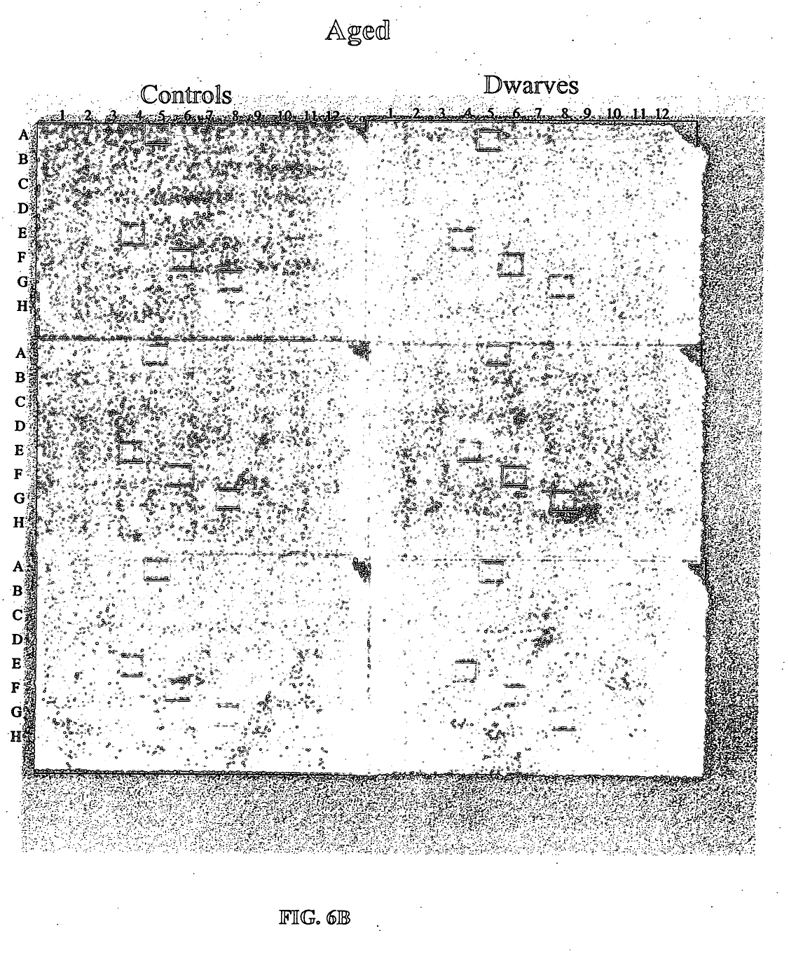

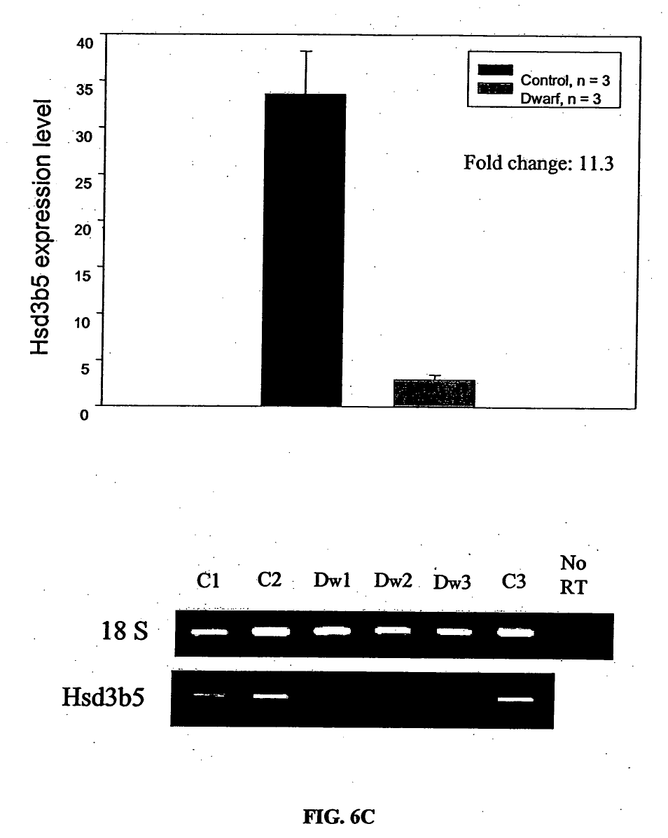

Analysis of mitochondrial gene expression in livers of young Snell dwarf mouse mutants and aged Snell dwarf mouse mutants was performed (FIG. 6A, FIG. 6B, Table 6). The Snell dwarf mouse served as a genetic model of longevity because of its increased life-span (40%). These analyses of mitochondrial gene expression were designed to determine whether there are specific changes or differences in mitochondrial gene expression associated with longevity. Differences in mitochondrial gene activity in livers of 4 young control, and 4 young (long-lived) Snell dwarf mouse mutants were observed. The mitochondrial genes that change in the young dwarfs are: A2—acyl CoA dehydrogenase; A5—5-aminolevulinate synthase; D8—3-beta hydroxy-5-ene-steroid dehydrogenase (Hsd3b1); D11, heat shock protein 70; E4—carbonyl reductase (NADPH); F6—sterol carrier protein X; G8—3-beta hydroxy-5-ene-steroid dehydrogenase (Hsd3b5). G7—GAPDH served as a positive control.

The differences in mitochondrial gene activity in livers of 3 aged controls and 3 aged long-lived Snell dwarf mouse mutants were also analyzed. The mitochondrial genes that change in the aged dwarfs are: A2, acyl-CoA dehydrogenase; A5—5-aminolevulinate synthase; E4—carbonyl reductase (NADPH); F6—sterol carrier protein X; and G8—Hsd3b5.

Overall, the data suggest that there are major differences in steroid metabolism between aged control and aged long-lived dwarf mutants. FIG. 6C shows RT-PCR analysis of Hsd3b5 (G8) expression levels in the control versus dwarf Snell mice. mRNA levels confirmed that the levels of this gene are significantly decreased in the liver mitochondria of the aged dwarf.

TABLE 6
Microarray template for FIGS. 6A and 6B

| | | | | | | | | | | | | |
|---|---|---|---|---|---|---|---|---|---|---|---|---|
| A | 1 | 2 | 3 | 4 | 5 | 6 | 7 | 8 | 9 | 10 | 11 | 12 |
| B | 13 | 14 | 15 | 16 | 17 | 18 | 19 | 20 | 21 | 22 | 23 | 24 |
| C | 25 | 26 | 27 | 28 | 29 | 30 | 31 | 32 | 33 | 34 | 35 | 36 |
| D | 37 | 38 | 39 | 40 | 41 | 42 | 43 | 44 | 45 | 46 | 47 | 48 |
| E | 49 | 50 | 51 | 52 | 53 | 54 | 55 | 56 | 57 | 58 | 59 | 60 |
| F | 61 | 62 | 63 | 64 | 65 | 66 | 67 | 68 | 69 | 70 | 71 | 72 |
| G | 73 | 74 | 75 | 76 | 77 | 78 | 79 | 80 | 81 | 82 | 83 | 84 |
| H | 85 | 86 | 87 | 88 | 89 | 90 | 91 | 92 | 93 | 94 | 95 | 96 |

| spot # | Gene name | Mitop/genbank | Description 10 ng/spot, 0.1 μM each primer |
|---|---|---|---|
| 1 | Acad1 | ACDL_MOUSE | Acyl-CoA dehydrogenase, long-chain specific precursor (LCAD) |
| 2 | Acadm | A55724 | Acyl-CoA dehydrogenase, medium-chain specific precursor |
| | | | (MCAD) |
| 3 | Acads | I49605 | Acyl-CoA dehydrogenase, short-chain specific precursor |
| 4 | Aif | AF100927 | Apoptosis-inducing factor |
| 5 | Alas2 | SYMSAL | 5-aminolevulinate synthase precursor |
| 6 | Aldh2 | I48966 | aldehyde dehydrogenase (NAD+) 2 precursor |
| 7 | Ant1 | S37210 | ADP, ATP carrier protein, heart isoform T1 |
| 8 | Ant2 | S31814 | ADP, ATP carrier protein, fibroblast isoform 2 |
| 9 | Aop1; Aop2 | JQ0064 | MER5 protein |
| 10 | Atp5a1 | JC1473 | H+-transporting ATP synthase chain alpha |
| 11 | Atp5g1 | ATPL_MOUSE | ATP synthase lipid-binding protein P1 precursor (protein 9) |
| 12 | Atp7b | U38477 | Probable copper transporting P-type ATPase |
| 13 | Bax | BAXA_MOUSE | apoptosis regulator BAX, membrane isoform alpha |
| 14 | Bckdha | S71881 | branched chain alpha-ketoacid dehydrogenase chain E1-alpha |
| 15 | Bckdhb | S39807 | 3-methyl-2-oxobutanoate dehydrogenase (lipoamide) |
| 16 | Bcl2 | B25960 | transforming protein bcl-2-beta |
| 17 | Bzrp | A53405 | peripheral-type benzodiazepine receptor 1 |
| 18 | Car5 | S12579 | carbonate dehydratase, hepatic |
| 19 | Ckmt1 | S24612 | creatine kinase |
| 20 | Cox4 | S12142 | cytochrome c oxidase chain IV precursor |
| 21 | Cox7a2 | I48286 | cytochrome C oxydase polypeptide VIIa-liver/heart precursor |
| 22 | Cox8a | COXR_MOUSE | cytochrome c oxidase chain VIII |
| 23 | Cpo | A48049 | Coproporphyrinogen oxidase |
| 24 | Cpt2 | A49362 | carnitine O-palmitoyltransferase II precursor |
| 25 | Crat | CACP_MOUSE | carnitine O-acetyltransferase (carnitine acetylase) |
| 26 | Cycs | CCMS | cytochrome C, somatic |
| 27 | Dbt | S65760 | dihydrolipoamide transacylase precursor |
| 28 | Dci | S38770 | 3,2-trans-enoyl-CoA isomerase, mitochondrial precursor |
| 29 | Dld | 1E+05 | dihydrolipoamide dehydrogenase (E3) |
| 30 | Fdx1 | S53524 | adrenodoxin precursor |
| 31 | Fdxr | S60028 | ferredoxin--NADP+ reductase precursor |
| 32 | | | Blank |
| 33 | Fpgs | S65755 | Tetrahydrofolylpolyglutamate synthase precursor |
| 34 | Frda | S75712 | Friedreich ataxia |
| 35 | Gcdh | GCDH_MOUSE | Glutaryl-CoA dehydrogenase precursor (GCD) |
| 36 | Glud | S16239 | glutamate dehydrogenase (NAD(P)+) precursor |
| 37 | Got2 | S01174 | glutamate oxaloacetaate transaminase-2 |
| 38 | Hadh | JC4210 | 3-hydroxyacyl-CoA dehydrogenase, short chain-specific, precursor |
| 39 | Hccs | CCHL_MOUSE | cytochrome C-type heme lyase (CCHL) |
| 40 | Hk1 | A35244 | hexokinase I |
| 41 | Hmgc1 | HMGL_MOUSE | Hydroxymethylglutaryl-CoA lyase |
| 42 | Hmgcs2 | B55729 | Hydroxymethylglutaryl-CoA synthase, mitochondrial |
| 43 | Hsc70t | 96231 | heat shock protein cognate 70, testis |
| 44 | Hsd3b1 | 3BH1_MOUSE | 3-beta hydroxy-5-ene steroid dehydrogenase type I |
| 45 | Hsp60 | HHMS60 | heat shock protein 60 precursor |
| 46 | Hsp70-1 | Q61698 | heat shock protein, 70K (hsp68) (fragment) |
| 47 | Hsp74 | A48127 | heat shock protein 70 precursor |
| 48 | HspE1 | A55075 | chaperonin-10 |
| 49 | Idh2 | IDHP_MOUSE | isocitrate dehydrogenase (NADP) |
| 50 | Mimt44 | U69898 | TIM44 - mitochondrial inner membrane import subunit |
| 51 | Mor1 | DEMSMM | malate dehydrogenase precursor, mitochondrial |
| 52 | Cbr2 | A28053 | carbonyl reductase (NADPH) - mouse |
| 53 | Cox6a1 | COXD_MOUSE | cytochrome C oxydase polypeptide VIa-heart precursor |
| 54 | Mthfd | A33267 | Methylenetetrahydrofolate dehydrogenase (NAD+) |
| 55 | Mut | S08680 | methylmalonyl-CoA mutase alpha chain precursor |
| 56 | Nnt | S54876 | NAD(P)+ transhydrogenase (B-specific) precursor |
| 57 | Oat | XNMSO | ornithine--oxo-acid transaminase precursor |
| 58 | Oias1 | 25A1_MOUSE | (2′-5′)oligoadenylate synthetase 1 |
| 59 | Otc | OWMS | ornithine carbamoyltransferase precursor |
| 60 | Pcx | A47255 | pyruvate carboxylase |
| 61 | Pdha1 | S23506 | pyruvate dehydrogenase (lipoamide) |
| 62 | sdh1 | bc013509 | succinate dehydrogenase subunit b iron sulphur protein |
| 63 | Polg | DPOG_MOUSE | DNA polymerase gamma |
| 64 | sdh2 | xm_127445 | succinate dehydrogenase subunit a flavoprotein |
| 65 | sdhc | nm_025321 | succinate dehydrogenase integral membrane protein CII-3 |
| 66 | Scp2 | JU0157 | sterol carrier protein x |
| 67 | Sod2 | I57023 | superoxide dismutase (Mn) precursor |
| 68 | Star | A55455 | steroidogenic acute regulatory protein precursor, mitochondrial |
| 69 | Tfam | P97894 | mitochondrial transcription factor A - mouse |
| 70 | Tst | THTR_MOUSE | thiosulfate sulfurtransferase |
| 71 | Ung | UNG_MOUSE | uracil-DNA glycosylase |
| 72 | Vdac1 | 1E+05 | voltage-dependent anion channel 1 |
| 73 | Vdac2 | 1E+05 | voltage-dependent anion channel 2 |
| 74 | Vdac3 | 1E+05 | voltage-dependent anion channel 3 |
| 75 | Ywhaz | JC5384 | 14-3-3 protein zeta/delta |
| 76 | WS-3 | | |
| 77 | Skd3 | | |
| 78 | | L00923 | Myosin 1 |
| 79 | GAPDH | M32599 | Glyceraldehyde 3-phosphate dehydrogenase (G3PDH) |
| 80 | Hsd3b5 | L41519 | 3-keytosteroid reductase |
| 81 | APE 1 | P28352 | Apurinic/apyrimidinic endonuclease 1 |
| 82 | Ogdh | U02971 | 2-Oxoglutarate dehydrogenase E1 component |
| 83 | ACADV | U41497 | Acyl-Co A dehydrogenase very long chain |
| 84 | Slc1a1 | EAT3_MOUSE | Excitatory amino acid transporter 3 |
| 85 | Hprt | J00423 | Hypoxanthine phosphoribosyl transferase (HPRT) |
| 86 | PplA2 | D78647 | Phospholipase A2 |
| 87 | Cab45 | U45977 | Calcium-binding protein Cab45 |
| 88 | NRF1 | NM_010938 | Nuclear Respiratory Factor 1 |
| 89 | Cox5b | x53157 | Cytochrome C oxidase subunit Vb |
| 90 | Cox 6a2 | L06465 | Cytochrome C oxidase subunit Via liver precursor |
| 91 | Atp5k | S52977 | ATP snythase H+ transporting chain e |
| 92 | β-actin | X03672 | beta-actin |
| 93 | | M10624 | Murine ornithine decarboxylase (MOD) |
| 94 | Tom40 | | Mitochondrial outer membrane protein |
| 95 | Gpam | | Glycerol-3-phosphate acyltransferase |
| 96 | sdhd | xm_134803 | succinate dehydrogenase small subunit integral membrane protein |

### Example 6

### Mitochondrial Gene Expression In Heart Muscle Of Trypanosome Infected Mice

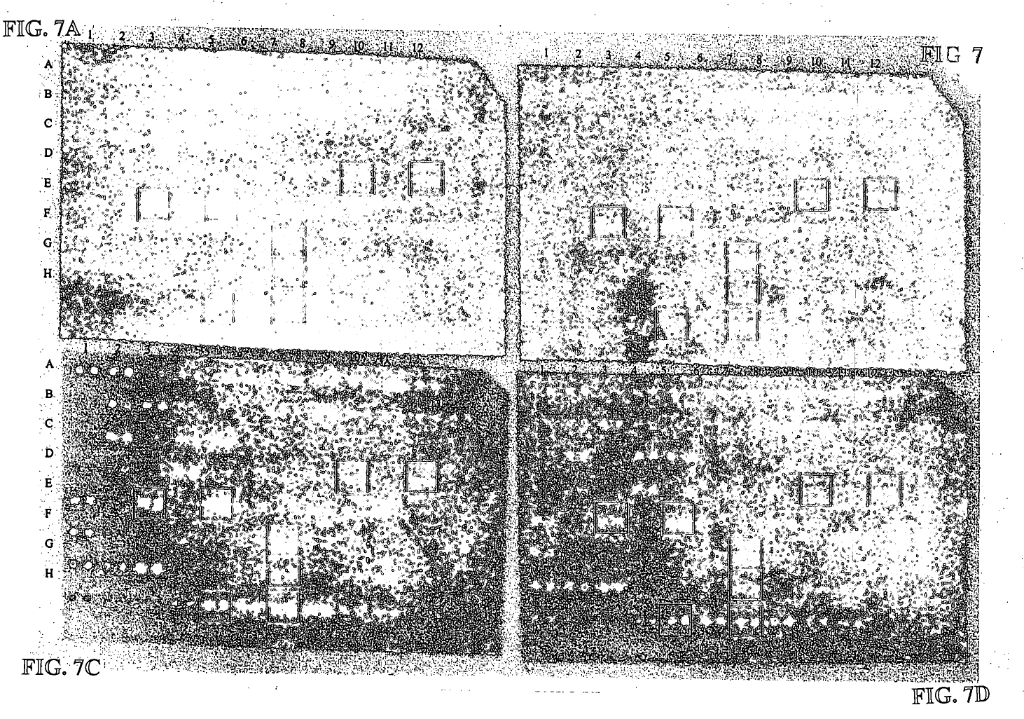

Trypanosome infections are chronic, and long after the initial infection the parasite accumulates in the heart and other organs. In the heart the parasite causes severe cardiovascular disease that results in heart failure. Thus, mitochondrial gene expression in heart muscle of trypanosome infected mice was analyzed (FIGS. 7A-7D, Table 7). The microarray for this analysis is composed of 96 genes of nuclear origin. The 13 genes encoded by the mitochondrial DNA were removed from the microarray and treated separately (see FIG. 5B, Table 5). The microarray analysis shows mRNA levels in a 4-month old mouse heart mitochondria 3 days postinfection and 37 days postinfection. When normalized to GAPDH (G7) and β-actin (H8) the data show an overall decrease in mitochondrial gene expression after 37 days postinfection. This decrease in mitochondrial function is a basic factor in trypanosome mediated cardiovascular pathology and ultimately leads to heart failure.

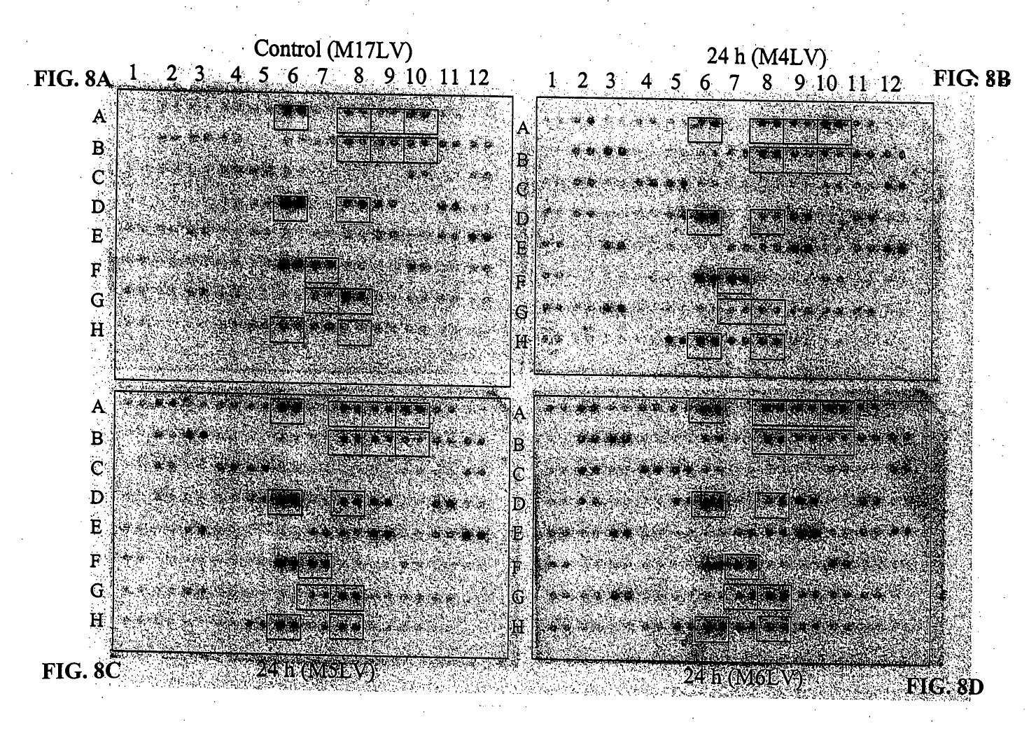

TABLE 7
Microarray template for FIGS. 7 and 8.

| | | | | | | | | | | | | |
|---|---|---|---|---|---|---|---|---|---|---|---|---|
| A | 1 | 2 | 3 | 4 | 5 | 6 | 7 | 8 | 9 | 10 | 11 | 12 |
| B | 13 | 14 | 15 | 16 | 17 | 18 | 19 | 20 | 21 | 22 | 23 | 24 |
| C | 25 | 26 | 27 | 28 | 29 | 30 | 31 | 32 | 33 | 34 | 35 | 36 |
| D | 37 | 38 | 39 | 40 | 41 | 42 | 43 | 44 | 45 | 46 | 47 | 48 |
| E | 49 | 50 | 51 | 52 | 53 | 54 | 55 | 56 | 57 | 58 | 59 | 60 |
| F | 61 | 62 | 63 | 64 | 65 | 66 | 67 | 68 | 69 | 70 | 71 | 72 |
| G | 73 | 74 | 75 | 76 | 77 | 78 | 79 | 80 | 81 | 82 | 83 | 84 |
| H | 85 | 86 | 87 | 88 | 89 | 90 | 91 | 92 | 93 | 94 | 95 | 96 |

| Spot # | Gene name | Mitop/genbank | Description 10 ng/spot, 0.1 μM each primer |
|---|---|---|---|
| 1 | Acadl | ACDL_MOUSE | Acyl-CoA dehydrogenase, long-chain specific precursor (LCAD) |
| 2 | Acadm | A55724 | Acyl-CoA dehydrogenase, medium-chain specific precursor (MCAD) |
| 3 | Acads | I49605 | Acyl-CoA dehydrogenase, short-chain specific precursor |
| 4 | Aif | AF100927 | Apoptosis-inducing factor |
| 5 | Alas2 | SYMSAL | 5-aminolevulinate synthase precursor |
| 6 | Aldh2 | I48966 | Aldehyde dehydrogenase (NAD+) 2 precursor |
| 7 | Ant1 | S37210 | ADP, ATP carrier protein, heart isoform T1 |
| 8 | Ant2 | S31814 | ADP, ATP carrier protein, fibroblast isoform 2 |
| 9 | Aop1; Aop2 | JQ0064 | MER5 protein |
| 10 | Atp5a1 | JC1473 | H+-transporting ATP synthase chain alpha |
| 11 | Atp5g1 | ATPL_MOUSE | ATP synthase lipid-binding protein P1 precursor (protein 9) |
| 12 | Atp7b | U38477 | Probable copper transporting P-type ATPase |
| 13 | Bax | BAXA_MOUSE | Apoptosis regulator BAX, membrane isoform alpha |
| 14 | Bckdha | S71881 | Branched chain alpha-ketoacid dehydrogenase chain E1-alpha |
| 15 | Bckdhb | S39807 | 3-methyl-2-oxobutanoate dehydrogenase (lipoamide) |
| 16 | Bcl2 | B25960 | Transforming protein bcl-2-beta |
| 17 | Bzrp | A53405 | Peripheral-type benzodiazepine receptor 1 |
| 18 | Car5 | S12579 | Carbonate dehydratase, hepatic |
| 19 | Ckmt1 | S24612 | Creatine kinase |
| 20 | Cox4 | S12142 | Cytochrome c oxidase chain IV precursor |
| 21 | Cox7a2 | I48286 | Cytochrome C oxydase polypeptide VIIa-liver/heart precursor |
| 22 | Cox8a | COXR_MOUSE | Cytochrome c oxidase chain VIII |
| 23 | Cpo | A48049 | Coproporphyrinogen oxidase |
| 24 | Cpt2 | A49362 | Carnitine O-palmitoyltransferase II precursor |
| 25 | Crat | CACP_MOUSE | Carnitine O-acetyltransferase (carnitine acetylase) |
| 26 | Cycs | CCMS | Cytochrome C, somatic |
| 27 | Dbt | S65760 | Dihydrolipoamide transacylase precursor |
| 28 | Dci | S38770 | 3,2-trans-enoyl-CoA isomerase, mitochondrial precursor |
| 29 | Dld | 1E+05 | Dihydrolipoamide dehydrogenase (E3) |
| 30 | Fdx1 | S53524 | Adrenodoxin precursor |
| 31 | Fdxr | S60028 | Ferredoxin--NADP+ reductase precursor |
| 32 | | | Blank |
| 33 | Fpgs | S65755 | Tetrahydrofolylpolyglutamate synthase precursor |
| 34 | Frda | S75712 | Friedreich ataxia |
| 35 | Gcdh | GCDH_MOUSE | Glutaryl-CoA dehydrogenase precursor (GCD) |
| 36 | Glud | S16239 | Glutamate dehydrogenase (NAD(P)+) precursor |
| 37 | Got2 | S01174 | Glutamate oxaloacetaate transaminase-2 |
| 38 | Hadh | JC4210 | 3-hydroxyacyl-CoA dehydrogenase, short chain-specific, precursor |
| 39 | Hccs | CCHL_MOUSE | Cytochrome C-type heme lyase (CCHL) |
| 40 | Hk1 | A35244 | Hexokinase I |
| 41 | Hmgc1 | HMGL_MOUSE | Hydroxymethylglutaryl-CoA lyase |
| 42 | Hmgcs2 | B55729 | Hydroxymethylglutaryl-CoA synthase, mitochondrial |
| 43 | Hsc70t | 96231 | Heat shock protein cognate 70, testis |
| 44 | Hsd3b1 | 3BH1_MOUSE | 3-beta hydroxy-5-ene steroid dehydrogenase type I |
| 45 | Hsp60 | HHMS60 | Heat shock protein 60 precursor |
| 46 | Hsp70-1 | Q61698 | Heat shock protein, 70K (hsp68) (fragment) |
| 47 | Hsp74 | A48127 | Heat shock protein 70 precursor |
| 48 | HspE1 | A55075 | Chaperonin-10 |
| 49 | Idh2 | IDHP_MOUSE | Isocitrate dehydrogenase (NADP) |
| 50 | Mimt44 | U69898 | TIM44-mitochondrial inner membrane import subunit |
| 51 | Mor1 | DEMSMM | Malate dehydrogenase precursor, mitochondrial |
| 52 | Cbr2 | A28053 | Carbonyl reductase (NADPH) - mouse |
| 53 | Cox6a1 | COXD_MOUSE | Cytochrome C oxydase polypeptide VIa-heart precursor |
| 54 | Mthfd | A33267 | Methylenetetrahydrofolate dehydrogenase (NAD+) |
| 55 | Mut | S08680 | Methylmalonyl-CoA mutase alpha chain precursor |
| 56 | Nnt | S54876 | NAD(P)+ transhydrogenase (B-specific) precursor |
| 57 | Oat | XNMSO | Ornithine--oxo-acid transaminase precursor |
| 58 | Oias1 | 25A1_MOUSE | (2′-5′)oligoadenylate synthetase 1 |
| 59 | Otc | OWMS | Ornithine carbamoyltransferase precursor |
| 60 | Pcx | A47255 | Pyruvate carboxylase |
| 61 | Pdha1 | S23506 | Pyruvate dehydrogenase (lipoamide) |
| 62 | Pdha1 | S23507 | Pyruvate dehydrogenase (lipoamide) |
| 63 | Polg | DPOG_MOUSE | DNA polymerase gamma |
| 64 | Ppox | S68367 | Protoporphyrinogen oxidase |
| 65 | Rpl23 | 1E+06 | L23 mitochondrial-related protein |
| 66 | Scp2 | JU0157 | Sterol carrier protein x |
| 67 | Sod2 | I57023 | Superoxide dismutase (Mn) precursor |
| 68 | Star | A55455 | Steroidogenic acute regulatory protein precursor, mitochondrial |
| 69 | Tfam | P97894 | Mitochondrial transcription factor A - mouse |
| 70 | Tst | THTR_MOUSE | Thiosulfate sulfurtransferase |
| 71 | Ung | UNG_MOUSE | Uracil-DNA glycosylase |
| 72 | Vdac1 | 1E+05 | Voltage-dependent anion channel 1 |
| 73 | Vdac2 | 1E+05 | Voltage-dependent anion channel 2 |
| 74 | Vdac3 | 1E+05 | Voltage-dependent anion channel 3 |
| 75 | Ywhaz | JC5384 | 14-3-3 protein zeta/delta |
| 76 | WS-3 | | |
| 77 | Skd3 | | |
| 78 | | L00923 | Myosin 1 |
| 79 | GAPDH | M32599 | Glyceraldehyde 3-phosphate dehydrogenase (G3PDH) |
| 80 | Hsd3b5 | L41519 | 3-keytosteroid reductase |
| 81 | APE 1 | P28352 | Apurinic/apyrimidinic endonuclease 1 |
| 82 | Ogdh | U02971 | 2-Oxoglutarate dehydrogenase E1 component |
| 83 | ACADV | U41497 | Acyl-Co A dehydrogenase very long chain |
| 84 | Slc1a1 | EAT3_MOUSE | Excitatory amino acid transporter 3 |
| 85 | Hprt | J00423 | Hypoxanthine phosphoribosyl transferase (HPRT) |
| 86 | PplA2 | D78647 | Phospholipase A2 |
| 87 | Cab45 | U45977 | Calcium-binding protein Cab45 |
| 88 | NRF1 | NM_010938 | Nuclear Respiratory Factor 1 |
| 89 | Cox5b | x53157 | Cytochrome C oxidase subunit Vb |
| 90 | Cox 6a2 | L06465 | Cytochrome C oxidase subunit Via liver precursor |
| 91 | Atp5k | S52977 | ATP snythase H+ transporting chain e |
| 92 | β-actin | X03672 | Beta-actin |
| 93 | | M10624 | Murine ornithine decarboxylase (MOD) |
| 94 | Tom40 | | Mitochondrial outer membrane protein |
| 95 | Gpam | | Glycerol-3-phosphate acyltransferase |
| 96 | Arg2 | | Arginase type II |

### Example 7

### Effects Of TBS Thermal Injury On Mouse Liver Mitochondrial Function

The effects of 40% TBS thermal injury on mouse liver mitochondrial function were examined (FIGS. 8A-8D, Table 7). In addition to a control (A), three livers from thermally injured mice 24 hours after burn were analyzed (B-D). The boxes indicate changes in levels of gene expression due to thermal injury. Some of the changes observed are as follows: A6—aldehyde dehydrogenase (NAD⁺)2; A8—ADP/ATP carrier protein, fibroblast isoform 2; A9—MER 5 protein; A10-H+ transporting ATP synthase chain α; B8—cytochrome c oxidase chain IV; D6—hydroxymethyl butyrly-CoA synthase; F7—super oxide dismustase (Mn); H6, cytochrome oxidase subunit Vb; H8, β-actin.

A microarray analysis of the expression of the 13 mitochondrial DNA encoded genes in livers of thermally injured mice was performed. FIG. 9 provides the results of the analysis of 3 individual mice 24 hours after thermal injury. The data clearly showed that expression of mitochondrial DNA encoded mRNAs is not affected by thermal injury. I, control; II-IV, 24 hours after thermal injury.

### Example 8

### Human Mitochondrial Microarray

In order to further demonstrate the capability of the present invention, a human DNA microarray was generated from PCR products using human cDNAs that code for mitochondrial proteins. These cDNAs were cloned into the pCR2.1 vector (Invitrogen). The genes were then attached to nylon membranes by cross linking with UV radiation and a hybridization study was conducted. The samples were labeled by reverse transcriptase incorporation of radiolabeled nucleotides and the results were observed by autoradiography. Intense and specific hybridization signals for specific target genes were detected at a number of positions indicating levels of transcript abundance. The data demonstrate successful and selective hybridization of human mitochondrial-related genes on the array. Table 8 represents an array of nuclear encoded genes for mitochondrial proteins and Table 9 represents an array of mitochondria encoded genes.

TABLE 8
Human Mito Chips (Nuclear Encoded Genes)
Plate 1

| | | | | | | | | | | | | |
|---|---|---|---|---|---|---|---|---|---|---|---|---|
| A | 1 | 2 | 3 | 4 | 5 | 6 | 7 | 8 | 9 | 10 | 11 | 12 |
| B | 13 | 14 | 15 | 16 | 17 | 18 | 19 | 20 | 21 | 22 | 23 | 24 |
| C | 25 | 26 | 27 | 28 | 29 | 30 | 31 | 32 | 33 | 34 | 35 | 36 |
| D | 37 | 38 | 39 | 40 | 41 | 42 | 43 | 44 | 45 | 46 | 47 | 48 |
| E | 49 | 50 | 51 | 52 | 53 | 54 | 55 | 56 | 57 | 58 | 59 | 60 |
| F | 61 | 62 | 63 | 64 | 65 | 66 | 67 | 68 | 69 | 70 | 71 | 72 |
| G | 73 | 74 | 75 | 76 | 77 | 78 | 79 | 80 | 81 | 82 | 83 | 84 |
| H | 85 | | | | | | | GAPDH | β-action | HPRT | MYOSIN | PPLA2 |

Plate 2

| | | | | | | | | | | | | |
|---|---|---|---|---|---|---|---|---|---|---|---|---|
| A | 86 | 87 | 88 | 89 | 90 | 91 | 92 | 93 | 94 | 95 | 96 | 97 |
| B | 98 | 99 | 100 | 101 | 102 | 103 | 104 | 105 | 106 | 107 | 108 | 109 |
| C | 110 | 111 | 112 | 113 | 114 | 115 | 116 | 117 | 118 | 119 | 120 | 121 |
| D | 122 | 123 | 124 | 125 | 126 | 127 | 128 | 129 | 130 | 131 | 132 | 133 |
| E | 134 | 135 | 136 | 137 | 138 | 139 | 140 | 141 | 142 | 143 | 144 | 145 |
| F | 146 | 147 | 148 | 149 | 150 | 151 | 152 | 153 | 154 | 155 | 156 | 157 |
| G | 158 | 159 | 160 | 161 | 162 | 163 | 164 | 165 | 166 | 167 | 168 | 169 |
| H | 170 | | | | | | | GAPDH | β-actin | HPRT | MYOSIN | PPLA2 |

Plate 3

| | | | | | | | | | | | | |
|---|---|---|---|---|---|---|---|---|---|---|---|---|
| A | 171 | 172 | 173 | 174 | 175 | 176 | 177 | 178 | 179 | 180 | 181 | 182 |
| B | 183 | 184 | 185 | 186 | 187 | 188 | 189 | 190 | 191 | 192 | 193 | 194 |
| C | 195 | 196 | 197 | 198 | 199 | 200 | 201 | 202 | 203 | 204 | 205 | 206 |
| D | 207 | 208 | 209 | 210 | 211 | 212 | 213 | 214 | 215 | 216 | 217 | 218 |
| E | 219 | 220 | 221 | 222 | 223 | 224 | 225 | 226 | 227 | 228 | 229 | 230 |
| F | 231 | 232 | 233 | 234 | 235 | 236 | 237 | 238 | 239 | 240 | 241 | 242 |
| G | 243 | 244 | 245 | 246 | 247 | 248 | 249 | 250 | 251 | 252 | 253 | 254 |
| H | | | | | | | | GAPDH | β-actin | HPRT | MYOSIN | PPLA2 |

| Spot No. | Gene Name | Accession No. | Description | Related Disease |
|---|---|---|---|---|
| 1 | ACAA.1 | D16294 | 3-oxoacyl-CoA thiolase | |
| 2 | ACADL | M74096 | long-chain-acyl-CoA dehydrogenase (LCAD) | LCAD deficiency |
| 3 | ACADM | AF251043 | acyl-CoA dehydrogenase precurser, medium-chain-specific | MCAD deficiency |
| 5 | ACADSB | U12778 | short/branched chain acyl-CoA dehydrogenase precursor | |
| 4 | ACADS | M26393 | acyl-CoA dehydrogenase precursor, short-chain-specific | SCAD deficiency |
| 6 | ACADVL | D43682 | acyl-CoA dehydrogenase, very-long-chain-specific-precursor | VLCAD deficiency |
| | | | (VLCAD) | |
| 7 | ACAT1 | D90228 | acetyl-CoA C-acetyltransferase 1 precursor | Deficiency of 3-ketothiolase |
| | | | | (3KTD) |
| 8 | ACO2 | U80040 | probable aconitate hydratase, mitochondrial (citrate | |
| | | | hydrolyase) | |
| 9 | AGAT | X86401 | glycine amidinotransferase precursor | |
| 10 | AK2 | U39945 | adenylate kinase isoenzyme 2, mitochondrial (ATP-AMP | |
| | | | transphosphorylase) | |
| 11 | AK3 | X60673 | nucleoside-triphosphate--adenylate kinase 3 | |
| 12 | ALDH2 | X05409 | aldehyde dehydrogenase (NAD+) 2 precursor | Alcohol intolerance, acute |
| 13 | ALDH4 | U24267 | Delta-1-pyrroline-5-carboxylate dehydrogenase precursor | Hyperprolinemia, type II |
| | | | | (HPII) |
| 14 | ALDH5 | M63967 | aldehyde dehydrogenase (NAD+) 5 precursor | |
| 15 | AMT | D13811 | glycine cleavage system T-protein precursor | Non-ketotic hyperglycinemia, |
| | | | (aminomethyltransferase) | type II (NKH2) |
| 16 | ANT2 | J02683 | ADP, ATP carrier protein T2 | |
| 17 | ANT3 | J03592 | ADP, ATP carrier protein T3 | |
| 18 | AOP1 | D49396 | mitochondrial thioredoxin-dependent peroxide reductase | |
| | | | precursor | |
| 19 | ARG2 | U75667 | arginase II precursor (non-hepatic arginase) (kidney type | |
| | | | arginase) | |
| 20 | ATP5A1 | X59066 | H+-transporting ATP synthase, mitochondrial F1 complex | |
| 21 | ATP5B | X05606 | H+-transporting ATP synthase, mitochondrial F1 complex | |
| 22 | ATP5D | X63422 | H+-transporting ATP synthase, F1 complex, δ chain | |
| | | | precursor | |
| 23 | ATP5F1 | X60221 | H+-transporting ATP synthase, complex F0, subunit B | |
| 24 | ATP5G3 | U09813 | ATP synthase, mitochondrial F0 complex, chain 9 (subunit | |
| | | | C) | |
| 25 | ATP5I | NM_007100 | H+-transporting ATP synthase, mitochondrial F0 complex | |
| 26 | ATP5J | M37104 | ATP synthase, mitochondrial F0 complex, subunit F6 | |
| 27 | ATP5O | X83218 | ATP synthase oligomycin sensitivity conferral protein | |
| | | | precursor | |
| 28 | BAX | L22473 | apoptosis regulator BAX, membrane isoform α | |
| 29 | BCAT2 | U68418 | thyroid-hormone aminotransferase | |
| 30 | BCL2L1 | Z23115 | BCL2-like 1 - human | |
| 31 | BCS1L | AF026849 | BCS1 (yeast homolog)-like - human | |
| 32 | BDH | M93107 | D-beta-hydroxybutyrate dehydrogenase precursor | |
| 33 | BID | AF042083 | BH3 interacting domain death agonist (BID) | |
| 34 | BNIP3L | AF079221 | bcl2/adenovirus e1b 19-kDa protein-interacting protein | |
| 35 | BZRP | M36035 | peripheral benzodiazepine receptor | |
| 36 | BZRP-S | L21950 | peripheral benzodiazepine receptor-related protein | |
| 37 | CACT | Y10319 | Carnitine-acylcarnitine translocase (CACT) | Carnitine-acylcarnitine |
| | | | | translocase deficiency |
| 38 | CASQ1 | S73775 | calsequestrin precursor, fast-twitch skeletal muscle | |
| 39 | CGI-114 | AF151872 | oligoribonuclease, mitochondrial precursor | |
| 40 | CKMT1 | XM_007535 | creatine kinase precursor | |
| 41 | CKMT2 | JO5401 | creatine kinase precursor, sarcomere-specific | |
| 42 | CLPX | AJ006267 | putative ATP-dependent CLP protease ATP-binding subunit | |
| | | | CPLX | |
| 43 | COQ7 | AF032900 | ubiquinone biosynthesis protein COQ7 (CLK1 homologue of | |
| | | | c. elegans) | |
| 44 | COX11 | AF044321 | cytochrome c oxidase assembly protein COX11 | |
| 45 | COX4 | X54802 | cytochrome-c oxidase chain IV precursor | |
| 46 | COX5A | NM_004255 | cytochrome-c oxidase chain Va precursor | |
| 47 | COX5B | M19961 | cytochrome-c oxidase chain Vb precursor | |
| 48 | COX6A2 | NM_005205 | cytochrome-c oxidase chain VIa precursor, cardiac | |
| 49 | COX6B | XM_009350 | cytochrome-c oxidase chain VIb | |
| 50 | COX7A1 | XM_009337 | cytochrome-c oxidase chain VIIa precursor, cardiac and | |
| | | | skeletal | |
| 51 | COX7RP | AB007618 | cytochrome-c oxidase subunit VIIA-related protein | |
| 52 | CPO | Z28409 | coproporphyrinogen oxidase | Hereditary coproporphyria |
| | | | | (HCP) |
| 53 | CPS1 | XM_010819 | carbamoyl-phosphate synthase (ammonia) precursor | Hyperammonemia, type I |
| 54 | CPT2 | M58581 | carnitine O-palmitoyltransferase II precursor | Carnitine O- |
| | | | | palmitoyltransferase II |
| | | | | deficiency |
| 55 | CRAT | X78706 | carnitine O-acetyltransferase precursor | Carnitine O-acetyltransferase |
| | | | | deficiency |
| 56 | CS | AF047042 | citrate synthase, mitochondrial | |
| 57 | CYB5 | NM_030579 | cytochrome b5, microsomal form | |
| 58 | CYC1 | NM_001916 | ubiquinol--cytochrome-c reductase cytochrome c1 precursor | |
| 59 | CYP11A1 | M14565 | cholesterol monooxygenase (side-chain-cleaving) | |
| | | | cytochrome P450e | |
| 60 | CYP3 | NM_005729 | peptidylprolyl isomerase 3 precursor | |
| 61 | DBT | X66785 | dihydrolipoamide S-(2-methylpropanoyl) transferase | Maple syrup urine disease |
| | | | precursoror | (MSUD) |
| 62 | DCI | Z25820 | dodecenoyl-CoA δ-isomerase precursor | |
| 63 | DECR | XM_005309 | 2,4-dienoyl-CoA reductase precursor | Deficiency of 2,4-dienoyl- |
| | | | | CoA reductase |
| 64 | DFN1 | U66035 | deafness dystonia protein | Mohr-Tranebjaerg syndrome |
| | | | | (MTS) |
| 65 | DIA1 | XM_010028 | cytochrome-b5 reductase | |
| 66 | DLAT_h | X13822 | dihydrolipoamide S-acetyltransferase heart | |
| 67 | DLD | J03620 | dihydrolipoamide dehydrogenase precursor | Dihydrolipoamide |
| | | | | dehydrogenase deficiency; |
| | | | | Leigh syndrome |
| 68 | DLST | XM_012353 | dihydrolipoamide S-succinyltransferase | |
| 69 | ECGF1 | M63193 | thymidine phosphorylase precursor (TDRPASE) | Myoneurogastrointestinal |
| | | | | encephalopathy syndrome |
| | | | | (MNGIE) |
| 70 | ECHS1 | XM_005677 | enoyl-CoA hydratase, mitochondrial | |
| 71 | EFE2 | X92762 | tafazzins protein | Barth syndrome |
| 72 | EFTS | AF110399 | mitochondrial elongation factor TS precursor (EF-TS) | |
| 73 | ENDOG | XM_005364 | endonuclease G, mitochondrial | |
| 74 | ETFA | XM_007626 | electron transfer flavoprotein alpha chain precursor | Glutaric aciduria, type IIa |
| | | | | (GAIIa) |
| 75 | ETFDH | NM_004453 | electron transfer flavoprotein dehydrogenase | Glutaric aciduria, type IIc |
| | | | | (GAIIc) |
| 76 | FACL1 | XM_010921 | long-chain-fatty-acid--CoA ligase 1 (palmitoyl-CoA ligase) | |
| 77 | FACL2 | NM_021122 | long-chain-fatty-acid--CoA ligase 2 | |
| 78 | FDX1 | M34788 | adrenodoxin precursor | |
| 79 | FDXR | J03826 | ferredoxin--NADP+ reductase, long form, precursor | |
| 80 | GCDH | U69141 | glutaryl-CoA dehydrogenase precursor (GCD) | Glutaric aciduria, type I (GA- |
| | | | | I) |
| 81 | GCSH | XM_010661 | glycine cleavage system protein H precursor | Non-ketotic hyperglycinemia, |
| | | | | type III (NKH3) |
| 82 | GK | XM_010221 | glycerol kinase (ATP: glycerol 3-phosphotransferase) | Glycerol kinase deficiency |
| | | | | (GKD) |
| 83 | GLDC | XM_011805 | glycine dehydrogenase (decarboxylating) precursor | Non-ketotic hyperglycinemia, |
| | | | | type I (NKH1) |
| 84 | GLUD1 | X07769 | glutamate dehydrogenase (NAD(P)+) precursor | |
| 85 | GOT2 | M22632 | aspartate transaminase precursor | |
| 86 | GPD2 | XM_002442 | glycerol-3-phosphate dehydrogenase | Diabetes mellitus, type II |
| | | | | (NIDDM) |
| 87 | GST12 | J03746 | glutathione transferase, microsomal | |
| 88 | HADHA | NM_000182 | long-chain-fatty-acid beta-oxidation multienzyme complex | Trifunctional enzyme |
| | | | alpha | deficiency; Maternal acute |
| | | | | fatty liver of pregnancy |
| | | | | (AFLP) |
| 89 | HADHB | NM_000183 | long-chain-fatty-acid beta-oxidation multienzyme complex | Trifunctional enzyme |
| | | | beta | deficiency |
| 90 | HCCS | U36787 | cytochrome c-type heme lyase (holocytochrome-c synthase) | |
| | | | human) human | |
| 91 | HK1 | X66957 | hexokinase I | |
| 92 | HK2 | NM_000189 | hexokinase II | Diabetes mellitus, type II |
| | | | | (NIDDM) |
| 93 | HLCS | XM_009757 | biotin--[methylmalonyl-CoA-carboxyltransferase] ligase | Biotin-responsive multiple |
| | | | | carboxylase deficiency |
| 94 | HMGCL | L07033 | hydroxymethylglutaryl-CoA lyase | Hydroxymethylglutaricaciduria |
| | | | | (HMGCL) |
| 95 | HSD3B1 | M27137 | 3-beta hydroxy-5-ene steroid dehydrogenase type I | Severe depletion of steroid |
| | | | | formation |
| 96 | HSPA1L | M11717 | heat shock protein HSP70 | |
| 97 | HSPA9 | L15189 | mitochondrial hsp70 precursor | |
| 98 | HSPD1 | M22382 | heat shock protein 60 precursor | |
| 99 | HSPE1 | X75821 | heat shock protein 10 | |
| 100 | HTOM34P | U58970 | Human putative outer mitochondrial membrane 34 kDa | |
| | | | translocase | |
| 101 | HTOM | AF026031 | putative mitochondrial outer membrane protein import | |
| | | | receptor | |
| 102 | IDH2 | X69433 | isocitrate dehydrogenase (NADP+) precursor | |
| 103 | IDH3A | U07681 | NAD(H)-specific isocitrate dehydrogenase α chain | |
| | | | precursorursor | |
| 104 | IDH3B | U49283 | isocitrate dehydrogenase (NAD), mitochondrial subunit β | |
| 105 | IDH3G | Z68907 | isocitrate dehydrogenase (NAD), mitochondrial subunit γ | |
| 106 | IVD | M34192 | isovaleryl-CoA dehydrogenase precursor | Isovaleric acidemia (IVA) |
| 107 | KIAA0016 | D13641 | Mitochondrial import receptor subunit TOM20 homolog | |
| 108 | KIAA0028 | D21851 | Probable leucyl-tRNA synthetase | |
| 109 | KIAA0123 | D50913 | mitochondrial processing peptidase α subunit precursor | |
| 110 | LOC51081 | AF077042 | ribosomal protein S7 small chain precursor | |
| 111 | LOC51189 | AB029042 | ATPase inhibitor precursor | |
| 112 | MAOA | M68840 | amine oxidase (flavin-containing) A | Brunner's syndrome |
| 113 | MAOB | XM_010261 | amine oxidase (flavin-containing) B | |
| 114 | MDH2 | XM_004905 | malate dehydrogenase mitochondrial precursor (fragment) | |
| 115 | ME2.1 | X79440 | malate dehydrogenase (oxaloacetate-decarboxylating) | |
| 116 | ME2 | M55905 | malate dehydrogenase (NAD+) precursor | |
| 117 | MFT | AF283645 | folate transporter/carrier | |
| 118 | MIPEP | U80034 | mitochondrial intermediate peptidase | |
| 119 | MLN64 | D38255 | MLN 64 protein (steroidogenic acute regulatory protein | |
| | | | related) | |
| 120 | MMSDH | M93405 | methylmalonate-semialdehyde dehydrogenase (acylating) | Methylmalonate semialdehyde |
| | | | | dehydrogenase deficiency |
| | | | | (MMSDHD) |
| 121 | MRRF | AF072934 | mitochondrial ribosome recycling factor 1 | |
| 122 | MTABC3 | AF076775 | mammalian mitochondrial ABC protein 3 | |
| 123 | MTCH1 | AF176006 | mitochondrial carrier homolog 1 isoform a | |
| 124 | MTCH2 | AF176008 | mitochondrial carrier homolog 2 | |
| 125 | MTERF | Y09615 | transcription termination factor | |
| 126 | MTHFD1 | J04031 | methylenetetrahydrofolate dehydrogenase (NADP+) | |
| 127 | MTHFD2 | X16396 | methylenetetrahydrofolate dehydrogenase (NAD+) | |
| 128 | MTIF2 | L34600 | translation initiation factor IF-2 precursor | |
| 129 | MTRF1 | AF072934 | mitochondrial translational release factor 1 | |
| 130 | MTX1 | XM_002192 | metaxin 1 - human | |
| 131 | MTX2 | XM_002547 | metaxin 2 - human | |
| 132 | MUT | M65131 | methylmalonyl-CoA mutase precursror (MCM) | Methylmalonic acidemia |
| | | | | (MUT-, MUT0 type) |
| 133 | MUTYH | U63329 | mutY (E. coli) homolog - human | |
| 134 | NDUFA10 | AF087661 | NADH dehydrogenase (ubiquinone) 1 α subcomplex, | |
| | | | 10 (42 KD) | |
| 136 | NDUFA2 | AF047185 | NADH dehydrogenase (ubiquinone) 1 α subcomplex, | |
| | | | 2 (8 kD) | |
| 137 | NDUFA4 | U94586 | NADH dehydrogenase (ubiquinone) 1 α subcomplex, | |
| | | | 4 (9 kD) | |
| 138 | NDUFA5 | U53468 | NADH dehydrogenase (ubiquinone) 1 α subcomplex, | |
| | | | 5 (13 kD) | |
| 139 | NDUFA6 | XM_010025 | NADH dehydrogenase (ubiquinone) 1 α subcomplex, | |
| | | | 6 (14 kD) | |
| 140 | NDUFA7 | NM_005001 | NADH dehydrogenase (ubiquinone) 1 α subcomplex, | |
| | | | 7 (14.5 kD) | |
| 141 | NDUFA8 | AF044953 | NADH dehydrogenase (ubiquinone) 1 α subcomplex, | |
| | | | 8 (19 KD) | |
| 142 | NDUFAB1 | AF087660 | acyl carrier protein, mitochondrial precursor (ACP) | |
| 143 | NDUFB1 | AF054181 | NADH dehydrogenase (ubiquinone) 1 β subcomplex, | |
| | | | 1 (7 KD) | |
| 144 | NDUFB2 | XM_004607 | NADH dehydrogenase (ubiquinone) 1 β subcomplex, | |
| | | | 2 (8 KD) | |
| 145 | NDUFB3 | NM_002491 | NADH dehydrogenase (ubiquinone) 1 β subcomplex, | |
| | | | 3 (12 KD) | |
| 146 | NDUFB4 | AF044957 | NADH dehydrogenase (ubiquinone) 1 β subcomplex, | |
| | | | 4 (15 KD) | |
| 147 | NDUFB5 | AF047181 | NADH dehydrogenase (ubiquinone) 1 β subcomplex, | |
| | | | 5 (16 KD) | |
| 148 | NDUFB6 | XM_005532 | NADH dehydrogenase (ubiquinone) 1 β subcomplex, | |
| | | | 6 (17 KD) | |
| 149 | NDUFB7 | M33374 | NADH dehydrogenase (ubiquinone) B18 subunit (Complex | |
| | | | I-B18) | |
| 150 | NDUFB8 | XM_005701 | NADH dehydrogenase (ubiquinone) 1 β subcomplex, | |
| | | | 8 (19 kD) | |
| 151 | NDUFB9 | S82655 | NADH dehydrogenase (ubiquinone) 1 β subcomplex, | |
| | | | 9 (22 kD) | |
| 152 | NDUFC2 | AF087659 | NADH dehydrogenase (ubiquinone) 1, subcomplex | |
| | | | unknown, 2(14.5 kD) | |
| 153 | NDUFS2 | AF050640 | NADH dehydrogenase (ubiquinone) Fe—S protein 2 (49 kD) | |
| 154 | NDUFS3 | AF067139 | NADH dehydrogenase (ubiquinone) 30K chain precursor | |
| 155 | NDUFS5 | AF020352 | NADH dehydrogenase (ubiquinone) Fe—S protein 5 (15 kD) | |
| 156 | NDUFS6 | AF044959 | NADH dehydrogenase (ubiquinone) 13 kD-A subunit | |
| | | | precursor | |
| 157 | NDUFS7 | NM_024407 | NADH dehydrogenase (ubiquinone) Fe—S protein 7 (20 kD) | Leigh syndrome |
| 158 | NDUFS8 | U65579 | NADH dehydrogenase (ubiquinone) 23 kD subunit precursor | Leigh syndrome |
| 159 | NDUFV1 | AF053070 | NADH dehydrogenase (ubiquinone) 51K chain precursor | Alexander disease; Leigh |
| | | | (fragment) | syndrome |
| 160 | NDUFV2 | M22538 | NADH dehydrogenase (ubiquinone) 24K chain precursor | |
| 161 | NDUFV3 | XM_009784 | NADH dehydrogenase (ubiquinone) 9 kD subunit precursor | |
| 162 | NIFS | XM_009457 | cysteine desulfurase (homolog of nitrogen-fixing bacteria) | |
| 163 | NME4 | Y07604 | nucleosid diphosphate kinase (NDP kinase) | |
| 164 | NNT-PEN | U40490 | NAD(P)+ transhydrogenase (B-specific) precursor | |
| 165 | NOC4 | XM_008056 | neighbor of COX4 (NOC4) | |
| 166 | NRF1 | NM_005011 | nuclear respiratory factor 1 | |
| 167 | NTHL1 | AB001575 | endonuclease III (E. coli) homolog | |
| 168 | OAT | M23204 | ornithine--oxo-acid transaminase precursor | Ornithinemia with gyrate |
| | | | | atrophy (GA) |
| 169 | OGDH | D10523 | oxoglutarate dehydrogenase (lipoamide) precursor | Deficiency of α-ketoglutarate |
| | | | | dehydrogenase |
| 170 | OGG1 | U96710 | 8-oxoguanine DNA glycosylase | |
| 171 | OIAS | X02874 | (2′-5′) oligoadenylate synthetase E16 | |
| 172 | OPA1 | XM_039926 | Optic atrophy 1 protein, KIAA0567 | Optic atrophy (OPA1) |
| 173 | OTC | K02100 | ornithine carbamoyltransferase precursor | Hyperammonemia, type II |
| 174 | OXA1L | X80695 | OXA1 homolog | |
| 175 | OXCT | U62961 | Succinyl-CoA:3-ketoacid-coenzyme A transferase precursor | Deficiency of Succinyl- |
| | | | | CoA:3-oxoacid-CoA |
| | | | | transferase |
| 176 | P43-LSB | S75463 | mitochondrial elongation factor-like protein P43 | |
| 177 | PCCA | X14608 | propionyl-CoA carboxylase α chain precursor | Propionic acidemia, type I |
| | | | | (PA-1) |
| 178 | PCCB | XM_051992 | propionyl-CoA carboxylase β chain precursor | Propionic acidemia, type II |
| | | | | (PA-2) |
| 179 | PCK2 | S69546 | phosphoenolpyruvate carboxykinase (GTP) precursor | Hypoglycemia and liver |
| | | | | impairment |
| 180 | PC | U04641 | pyruvate carboxylase precursor | Deficiency of pyruvate |
| | | | | carboxylase, type I and II |
| 181 | PDHA1 | J03503 | pyruvate dehydrogenase (lipoamide) α chain precursor | Pyruvate dehydrogenase |
| | | | | deficiency; Leigh syndrome |
| 182 | PDHA2 | M86808 | pyruvate dehydrogenase (lipoamide) α chain precursor, testis | |
| 183 | PDK1 | L42450 | pyruvate dehydrogenase kinase isoform 1 | |
| 184 | PDK2 | L42451 | pyruvate dehydrogenase kinase isoform 2 | |
| 185 | PDK3 | L42452 | pyruvate dehydrogenase kinase isoform 3 | |
| 186 | PDK4 | U54617 | pyruvate dehydrogenase kinase isoform 4 | |
| 187 | PDX1 | U82328 | pyruvate dehydrogenase complex protein X subunit | Pyruvate dehydrogenase |
| | | | precursor | deficiency |
| 188 | PEMT | AF176807 | phosphatidylethanolamine N-methyltransferase (PEMT) | |
| 189 | PET112L | AF026851 | probable glutamyl-tRNA(gln) amidotransferase subunit b | |
| 190 | PHC | XM_039620 | phosphate carrier isoform A (alternatively spliced, exonIIIA) | |
| 191 | PLA2G2A | M22430 | phospholipase A2, group IIA, platelet, synovial fluid | |
| 192 | PLA2G4 | M72393 | phospholipase A2, cytosolic, group IV | |
| 193 | PLA2G5 | U03090 | phospholipase A2, group V | |
| 194 | PMPCB | AF054182 | mitochondrial processing peptidase β subunit precursor | |
| 195 | POLG2 | U94703 | mitochondrial DNA polymerase accessory subunit | |
| 196 | POLG | X98093 | DNA polymerase γ (mitochondrial DNA polymerase | |
| | | | catalytic subunit | |
| 197 | POLRMT | U75370 | mitochondrial RNA polymerase (DNA directed) | |
| 198 | PPOX | D38537 | protoporphyrinogen oxidase (PPO) | Porphyria variegata (VP) |
| 199 | PRAX-1 | AF039571 | benzodiazepine receptor-associated protein 1 | |
| 200 | PRDX5 | AF110731 | Peroxiredoxin 5 (antioxidant enzyme B166) | |
| 201 | PYCR1 | M77836 | pyrroline-5-carboxylate reductase | |
| 202 | RPL23L | Z49254 | mitochondrial 60S ribosomal protein L23 | |
| 203 | RPML12 | X79865 | mitochondrial 60S ribosomal protein L7/L12 precursor | |
| 204 | RPML3 | X06323 | ribosomal protein L3 precursor | |
| 205 | RPMS12 | Y11681 | mitochondrial 40S ribosomal protein S12 precursor | |
| 206 | SCHAD | X96752 | 3-hydroxyacyl-CoA dehydrogenase, short chain-specific, | |
| | | | precursor | |
| 207 | SCO2 | AF177385 | SCO2 homolog of S. cerevisiae | Fatal infantile |
| | | | | cardioencephalomyopathy due |
| | | | | to Cox deficiency |
| 208 | SCP2 | M55421 | sterol carrier protein 2 | |
| 209 | SDH1 | U17248 | succinate dehydrogenase (ubiquinone) 27K iron-sulfur | |
| | | | protein | |
| 210 | SDH2 | L21936 | succinate dehydrogenase (ubiquinone) flavoprotein precursor | Leigh syndrome; Deficiency |
| | | | | of succinate dehydrogenase |
| 211 | SDHC | D49737 | succinate dehydrogenase (ubiquinone) cytochrome b large | Hereditary paraganglioma, |
| | | | subunit | type III (PGL3) |
| 212 | SDHD | AB006202 | succinate dehydrogenase (ubiquinone) cytochrome b small | Hereditary paraganglioma, |
| | | | subunit | type I (PGL1) |
| 213 | SerRSmt | AB029948 | seryl-tRNA synthetase | |
| 214 | SHMT2 | NM_005412 | glycine hydroxymethyltransferase precursor | |
| 215 | SLC20A3 | U25147 | tricarboxylate transport protein precursor | |
| 216 | SLC25A12 | Y14494 | mitochondrial carrier protein aralar 1 | |
| 217 | SLC25A16 | M31659 | mitochondrial solute carrier protein homolog | |
| 218 | SLC25A18 | AY008285 | solute carrier SLC25A18 | |
| 219 | SLC9A6 | AF030409 | sodium/hydrogen exchanger 6 (Na(+)H(+) exchanger | |
| 220 | SOD2 | X14322 | superoxide dismutase (Mn) precursor | |
| 221 | SSBP | M94556 | single-stranded mitochondrial DNA-binding protein | |
| | | | precursor | |
| 222 | SUCLA2 | XM_012310 | succinyl-CoA ligase (ADP_forming), β-chain precursor | |
| 223 | SUCLG1 | NM_003849 | succinyl-CoA ligase (GDP_forming), α-chain precursor | |
| 224 | SUCLG2 | AF058954 | succinyl-CoA ligase (GDP_forming), β-chain precursor | |
| 225 | SUOX | XM_006727 | sulfite oxidase precursor, mitochondrial | Sulfocysteinuria |
| 226 | SUPV3L1 | XM_005981 | putative ATP-dependent mitochondrial RNA-helicase | |
| 227 | SURF1 | NM_003172 | Surfeit locus protein 1 | Leigh syndrome |
| 228 | TAT | NM_000353 | tyrosine transaminase (EC 2.6.1.5) | Tyrosine transaminase |
| | | | | deficiency, type II (Richner- |
| | | | | Hanhart syndrome) |
| 229 | TCF6L1 | M62810 | transcription factor 1 precursor | |
| 230 | TID1 | AF061749 | tumorous imaginal discs homolog precursor (HTID-1) | |
| 231 | TIM17B | AF034790 | translocase of inner mitochondrial membrane 17 (yeast) | |
| | | | homolog B | |
| 232 | TIM17 | AF106622 | translocase of inner mitochondrial membrane 17 (yeast) | |
| | | | homolog A | |
| 233 | TIM23 | AF030162 | inner mitochondrial membrane translocase TIM23 | |
| 234 | TIM44 | AF041254 | translocase of inner mitochondrial membrane 44 | |
| 235 | TK2 | U77088 | thymidine kinase | |
| 236 | TST | X59434 | thiosulfate sulfurtransferase | |
| 237 | TUFM | L38995 | translation elongation factor Tu precursor | |
| 238 | UCP2 | U82819 | uncoupling protein 2 | |
| 239 | UCP3 | U82818 | uncoupling protein 3 | |
| 240 | UCP4 | NM_004277 | uncoupling protein 4 | |
| 241 | UNG | X15653 | uracil-DNA glycosylase precursor | |
| 242 | UQCRB | NM_006294 | ubiquinone-binding protein QP-C | |
| 243 | UQCRC1 | NM_003365 | ubiquinol--cytochrome-c reductase core I protein | |
| 244 | UQCRC2 | NM_003366 | ubiquinol--cytochrome-c reductase core protein II | |
| 245 | UQCRFS1 | XM_012812 | ubiquinol--cytochrome-c reductase iron-sulfur subunit | Mitochondrial myopathy |
| | | | precursor | (MM) |
| 246 | UQCRH | NM_006004 | ubiquinol--cytochrome-c reductase 11K protein precursor | |
| 247 | UROS | AF230665 | uroporphyrinogen-III synthase | |
| 248 | VDAC1 | L06132 | voltage-dependent anion channel 1 | |
| 249 | VDAC2 | L06328 | voltage-dependent anion channel 2 | |
| 250 | VDAC3 | NM_005662 | voltage-dependent anion channel 3 | |
| 251 | WARS2 | XM_001388 | tryptophanyl-tRNA synthetase 2 | |
| 252 | WFS | AF084481 | Transmembrane protein | Diabetes mellitus and |
| | | | | insipidus with optic atrophy |
| | | | | and deafness (DIDMOAD); |
| | | | | Wolfram syndrome |
| 253 | YME1L1 | AJ132637 | ATP-dependent metalloprotease YME1 | |
| 254 | YWHAE | U28936 | 14-3-3 protein epsilon (mitochondrial import stimulation | |
| | | | factor) | |

TABLE 9
Human Mito Chip (Mitochondria encoded)

| Spot # | Genomic | Accession | Description | |
|---|---|---|---|---|
| 1 | MTCO1 | V00662 | Cytochrome-c oxidase chain I | |
| 2 | MTCO2 | V00662 | Cytochrome-c oxidase chain II | |
| 3 | MTCO3 | V00662 | Cytochrome-c oxidase chain III | |
| 4 | MTCYB | V00662 | Ubiquinol--cytochrome-c reductase cytochrome b | |
| 5 | MTND1 | J01415 | NADH dehydrogenase (ubiquinone) chain 1 | |
| 6 | MTND2 | J01415 | NADH dehydrogenase (ubiquinone) chain 2 | |
| 7 | MTND3 | J01415 | NADH dehydrogenase (ubiquinone) chain 3 | |
| 8 | MTND4 | J01415 | NADH dehydrogenase (ubiquinone) chain 4 | |
| 9 | MTND4L | J01415 | NADH dehydrogenase (ubiquinone) chain 4L | |
| 10 | MTND5 | J01415 | NADH dehydrogenase (ubiquinone) chain 5 | |
| 11 | MTND6 | J01415 | NADH dehydrogenase (ubiquinone) chain 6 | |
| 12 | MT-ATP 6 | J01415 | ATP synthase subunit 6 | |
| 13 | MT-ATP 8 | J01415 | ATP synthase subunit 8 | |
| 14 | MTRNR1 | J01415 | mitochondrial ribosomal RNA, 12S | Aminoglycoside-induced |
| | | | | deafness; Nonsyndromic deafness |
| 15 | MTRNR2 | J01415 | mitochondrial ribosomal RNA, 16S | Chloramphenicol |
| | | | | resistance; Alzheimer disease and |
| | | | | Parkinson disease (ADPD) |

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 4,800,159
U.S. Pat. No. 4,883,750
U.S. Pat. No. 5,143,854
U.S. Pat. No. 5,578,832
U.S. Pat. No. 5,837,832
U.S. Pat. No. 5,837,860
U.S. Pat. No. 5,861,242
Ames et al., Proc. Natl. Acad. Sci. USA 90: 7915-7922, 1993.
Anderson et al., Nature, 290:457-465, 1981.
Ballinger et al., Cir. Res., 86:960-966, 2000.
Beckman and Ames, Physiol. Rev., 78:547-581, 1998.
Beckman and Ames, Mutat. Res., 424:51-58, 1999.
British Appl. GB 2,202,328
Corral-Debrinski et al. Genomics, 23:471-476, 1994.
Corral-Debrinski et al., JAMA, 266:1812-1816, 1991.
Corral-Debrinski et al. Mutat. Res., 275:169-180, 1992a.
Corral-Debrinski et al. Nat. Genet., 2:324-329, 1992b.
European Appl. 329 822
European Appl. 320,308
Finkel and Holbrook, Nature, 408:239-247, 2000.
Fodor et al., Biochemistry, 30(33):8102-8108, 1991.
Frohman, In: PCR Protocols: A Guide To Methods And Applications, Academic Press, N.Y., 1990.
Golden and Melov, Mech. Aging Dev., 122:1577-1589, 2001.
Hacia et al., Nature Genet., 14:441-449, 1996.
Harmon, Am. J. Geriatric Soc., 20:145-147, 1972.
Horton et al., Neurology, 45:1879-188, 1995.
Hsieh et al., Mech. Age Dev., 123:1423-1435, 2002.
Innis et al., Proc. Natl. Acad. Sci. USA, 85(24):9436-9440, 1988.
Jun et al., Mol. Cell. Biol., 16:771-777, 1996.
Jun et al., Proc. Natl. Acad. Sci. USA, 91:6206-6210, 1994.
Kwoh et al., Proc. Nat. Acad. Sci. USA, 86: 1173, 1989.
Mandavilli et al., Mutation Res., 509:127-151, 2002.
Melov et al., Nucleic Acids Res., 23:4122-4126, 1995.
Melov et al., Nucleic Acids Res., 25:974-982, 1997.
Mitochondria and Free radicals In: Neurodegenerative Diseases, Beal et al. (Eds.), Wiley-Liss, NY, 1997.
Newton et al., Nucl. Acids Res., 21:1155-1162, 1993.
Ohara et al. Proc. Natl. Acad. Sci. USA, 86:5673-5677, 1989.
PCT Appl. PCT/US87/00880
PCT Appl. PCT/US89/01025
PCT Appl. WO 88/10315
PCT Appl. WO 89/06700
PCT Appl. WO 89/11548
PCT Appl. WO 90/15070
PCT Appl. WO 92/10092
Pease et al., Proc. NatL Acad. Sci. USA, 91:5022-5026, 1994.
Rasmussen et al., Anal. Biochem., 198:138-142, 1991.
Running et al., BioTechniques, 8:276-277, 1990.
Saito et al., J. Biol. Chem., 276:29307-29312, 2001.
Sambrook et al., In: Molecular cloning, Cold Spring Harbor Laboratory Press, Cold Spring Harbor, N.Y., 1989.
Sambrook et al., In: Molecular cloning, Cold Spring Harbor Laboratory Press, Cold Spring Harbor, N.Y., 2001.
Santos et al., Methods Mol. Biol., 197:159-176, 2002
Shoemaker et al., Nature Genetics, 14:450-456, 1996.
Shoffner et al., Cell, 61:931-937, 1990.
Shoffner et al., Genomics, 17:171-184, 1993.
Shoffner et al., Proc. Natl. Acad. Sci. USA, 86:7952-7956, 1989.
Ventura, B., et al., Biochim. Biophys. Acta, 1553:249-260, 2002.
Walker et al., Proc. Natl. Acad. Sci. USA, 89:392-396 1992.
Wallace et al., Cell, 55:601-610, 1988.
Wallace, Science, 283:1482-1488, 1999.
Waterston et al., Nature, 420, 520-562, 2002.
Wu et al., Genomics, 4:560-569, 1989.
Zhang et al., FEBS Lett., 297:34-38, 1992.

What is claimed is:

1. An array comprising nucleic acid molecules comprising a plurality of sequences, wherein the molecules are immobilized on a solid support and wherein at least 5% of the immobilized molecules are capable of hybridizing to mitochondrial-related nucleic acid sequences or complements thereof.

2. The array of claim 1, further defined as comprising at least 20 nucleic acid molecules.

3. The array of claim 1, further defined as comprising at least 40 nucleic acid molecules.

4. The array of claim 1, further defined as comprising at least 100 nucleic acid molecules.

5. The array of claim 1, further defined as comprising at least 200 nucleic acid molecules.

6. The array of claim 1, further defined as comprising at least 400 nucleic acid molecules.

7. The array of claim 1, wherein said nucleic acid molecules comprise cDNA sequences.

8. The array of claim 1, wherein each of said nucleic acid molecules comprises at least 17 nucleotides.

9. The array of claim 1, wherein the mitochondrial-related nucleic acid sequences are from a mammal.

10. The array of claim 9, wherein the mitochondrial-related nucleic acid sequences are from a primate.

11. The array of claim 9, wherein the mitochondrial-related nucleic acid sequences are from a human.

12. The array of claim 9, wherein the mitochondrial-related nucleic acid sequences are from a yeast.

13. The array of claim 9, wherein the mitochondrial-related nucleic acid sequences are from a mouse.

14. The array of claim 9, wherein the mitochondrial-related nucleic acid sequences are from Drosophila.

15. The array of claim 9, wherein the mitochondrial-related nucleic acid sequences are from the nematode, C. elegans.

16. The array of claim 1, wherein at least 25% of the immobilized molecules are capable of hybridizing to mitochondrial-related nucleic acid sequences or complements thereof.

17. The array of claim 1, wherein at least 35% of the immobilized molecules are capable of hybridizing to mitochondrial-related nucleic acid sequences or complements thereof.

18. The array of claim 1, wherein at least 50% of the immobilized molecules are capable of hybridizing to mitochondrial-related nucleic acid sequences or complements thereof.

19. The array of claim 1, wherein at least 75% of the immobilized molecules are capable of hybridizing to mitochondrial-related nucleic acid sequences or complements thereof.

20. The array of claim 1, wherein at least 85% of the immobilized molecules are capable of hybridizing to mitochondrial-related nucleic acid sequences or complements thereof.

21. The array of claim 1, wherein at least 95% of the immobilized molecules are capable of hybridizing to mitochondrial-related nucleic acid sequences or complements thereof.

22. The array of claim 1, wherein 100% of the immobilized molecules are capable of hybridizing to mitochondrial-related nucleic acid sequences or complements thereof.

23. The array of claim 1, wherein at least one of said mitochondrial-related nucleic acid sequences is encoded by a mitochondrial genome.

24. The array of claim 1, wherein the immobilized molecules are capable of hybridizing to at least 5 mitochondrial-related nucleic acid sequences or complements thereof.

25. The array of claim 1, wherein the immobilized molecules are capable of hybridizing to at least 10 mitochondrial-related nucleic acid sequences or complements thereof.

26. The array of claim 1, wherein the immobilized molecules are capable of hybridizing to at least 13 mitochondrial-related nucleic acid sequences or complements thereof.

27. The array of claim 1, wherein the immobilized molecules are capable of hybridizing to at least 20 mitochondrial-related nucleic acid sequences or complements thereof.

28. The array of claim 1, wherein the immobilized molecules are capable of hybridizing to at least 30 mitochondrial-related nucleic acid sequences or complements thereof.

29. The array of claim 1, wherein the immobilized molecules are capable of hybridizing to at least 60 mitochondrial-related nucleic acid sequences or complements thereof.

30. The array of claim 1, wherein the immobilized molecules are capable of hybridizing to at least 100 mitochondrial-related nucleic acid sequences or complements thereof.

31. The array of claim 1, wherein the immobilized molecules are capable of hybridizing to at least 200 mitochondrial-related nucleic acid sequences or complements thereof.

32. The array of claim 1, wherein the immobilized molecules are capable of hybridizing to at least 300, at least 500, or at least 1000 mitochondrial-related nucleic acid sequences or complements thereof.

33. The array of claim 1, wherein at least one of said mitochondrial-related nucleic acid sequences is encoded by a nuclear genome.

34. The array of claim 1, wherein at least one of said mitochondrial-related nucleic acid sequences is encoded by a mitochondrial genome.

35. A method for measuring the expression of one or more mitochondrial-related coding sequence in a cell or tissue, said method comprising:
a) contacting an array according to claim 1 with a sample of nucleic acids from the cell or tissue under conditions effective for mRNA or complements thereof from said cell or tissue to hybridize with the nucleic acid molecules immobilized on the solid support; and
b) detecting the amount of mRNA or complements thereof hybridizing to mitochondrial-related nucleic acid sequences or complements thereof.

36. The method of claim 35, wherein said detecting is carried out calorimetrically, fluorometrically, or radiometrically.

37. The method of claim 35, wherein the cell is a mammal cell.

38. The method of claim 35, wherein the cell is a primate cell.

39. The method of claim 35, wherein the cell is a human cell.

40. The method of claim 35, wherein the cell is a mouse cell.

41. The method of claim 35, wherein the cell is a yeast cell.

42. A method of screening an individual for a disease state associated with altered expression of one or more mitochondrial-related nucleic acid sequences comprising:
a) contacting an array according to claim 1 with a sample of nucleic acids from the individual under conditions effective for the mRNA or complements thereof from said individual to hybridize with the nucleic acid molecules immobilized on the solid support;
b) detecting the amount of mRNA or complements thereof hybridizing to mitochondrial-related nucleic acid sequences; and
c) screening the individual for a disease state by comparing the expression of said mitochondrial-related nucleic acid sequences detected with a pattern of expression of said mitochondrial-related nucleic acid sequences associated with said disease state.

43. The method of claim 42, wherein said disease state is a disease state as listed in Table 1.

44. The method of claim 43, wherein the disease state is cystic fibrosis, Alzheimer's disease, Parkinson's disease, ataxia, diabetes mellitus, multiple sclerosis or cancer.

45. The method of claim 42, wherein said detecting is carried out calorimetrically, fluorometrically, or radiometrically.

46. The method of claim 42, wherein the individual is a mammal.

47. The method of claim 42, wherein the individual is a primate.

48. The method of claim 42, wherein the individual is a human.

49. The method of claim 42, wherein the individual is a mouse.

50. The method of claim 42, wherein the individual is a an arthropod.

51. The method of claim 42, wherein the individual is a nematode.

52. A method of screening a compound for its affect on mitochondrial structure and/or function comprising:
a) contacting an array according to claim 1 with a sample of nucleic acids from a cell under conditions effective for the mRNA or complements thereof from said cell to hybridize with the nucleic acid molecules immobilized on the solid support, wherein the cell has previously been contacted with said compound under conditions effective to permit the compound to have an affect on mitochondrial structure and/or function;
b) detecting the amount of mRNA encoded by mitochondrial-related nucleic acid sequences or complements thereof that hybridizes with the nucleic acid molecules immobilized on the solid support; and
c) correlating the detected amount of mRNA encoded by mitochondrial-related nucleic acid molecules or complements thereof with the affect of the compound mitochondrial structure and/or function.

53. The method of claim 52, wherein the compound is a small molecule.

54. The method of claim 52, wherein the compound is formulated in a pharmaceutically acceptable carrier or diluent.

55. The method of claim 52, wherein the compound is an oxidative stressing agent or an inflammatory agent.

56. The method of claim 52, wherein the compound is a chemotherapeutic agent.

57. The method of claim 52, wherein said detecting is carried out calorimetrically, fluorometrically, or radiometrically.

58. A method for screening an individual for reduced mitochondrial function comprising:
a) contacting an array according to claim 1 with a sample of nucleic acids from a cell under conditions effective for the mRNA or complements thereof from said cell to hybridize with the nucleic acid molecules immobilized on the solid support;
b) detecting the amount of mRNA encoded by mitochondrial-related nucleic acid sequences or complements thereof that hybridizes with the nucleic acid molecules immobilized on the solid support; and
c) correlating the detected amount of mRNA or complements thereof with reduced mitochondrial function.

59. The method of claim 58, wherein said detecting is carried out calorimetrically, fluorometrically, or radiometrically.

60. The method of claim 58, wherein the individual is a mammal.

61. The method of claim 58, wherein the individual is a primate.

62. The method of claim 58, wherein the individual is a human.

63. The method of claim 58, wherein the individual is a mouse.

64. The method of claim 58, wherein the individual is an arthropod.

65. The method of claim 58, wherein the individual is a nematode.
